SEQUENCE LISTING (64 sequences)

SEQ ID NO: 1 - DNA, 20 bp, Artificial Sequence
  Synthetic
caaagcgcggtccagccccg

SEQ ID NO: 2 - DNA, 20 bp, Artificial Sequence
  Synthetic
caaactcagcaagcaaagcg

SEQ ID NO: 3 - DNA, 20 bp, Artificial Sequence
  Synthetic
gtgcaggctggtacttccaa

SEQ ID NO: 4 - DNA, 20 bp, Artificial Sequence
  Synthetic
cattgccatgttgagacaca

SEQ ID NO: 5 - DNA, 20 bp, Artificial Sequence
  Synthetic
ataaactgaggttatcacag

SEQ ID NO: 6 - DNA, 20 bp, Artificial Sequence
  Synthetic
cggggctggaccgcgctttg

SEQ ID NO: 7 - DNA, 20 bp, Artificial Sequence
  Synthetic
cgctttgcttgctgagtttg

SEQ ID NO: 8 - DNA, 20 bp, Artificial Sequence
  Synthetic
ttggaagtaccagcctgcac

SEQ ID NO: 9 - DNA, 20 bp, Artificial Sequence
  Synthetic
tgtgtctcaacatggcaatg

SEQ ID NO: 10 - DNA, 20 bp, Artificial Sequence
  Synthetic
ctgtgataacctcagtttat

SEQ ID NO: 11 - RNA, 20 nt, Artificial Sequence
  Synthetic
caaagcgcgguccagccccg

SEQ ID NO: 12 - RNA, 20 nt, Artificial Sequence
  Synthetic
caaacucagcaagcaaagcg

SEQ ID NO: 13 - RNA, 20 nt, Artificial Sequence
  Synthetic
gugcaggcugguacuuccaa

SEQ ID NO: 14 - RNA, 20 nt, Artificial Sequence
  Synthetic
cauugccauguugagacaca

SEQ ID NO: 15 - RNA, 20 nt, Artificial Sequence
  Synthetic
auaaacugagguuaucacag

SEQ ID NO: 16 - protein, 385 aa, Artificial Sequence
  Synthetic
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 17 - DNA, 7969 bp, Artificial Sequence
  Synthetic
agtgtcttccactcggtgcgtctctctaggagccgcgcgggaaggatgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtgactcggctaggtggggcaaggctgggcagtgtccgagagagcacccctctctgcatctgaccacgtgctacccccatgctggaggtgacatctcttacgcccaacccttccccactgcacacacctcagaggctgttcttggggccctacaccttgaggaggggcaggtaaactcctgtcctttacacattcggctccctggagccagactctggtcttctttgggtaaacgtgtgacgggggaaagccaaggtctggagaagctcccaggaacaatcgatggccttgcagcactcacacaggacccccttcccctaccccctcctctctgccgcaatacaggaacccccaggggaaagatgagcttttctaggctacaattttctcccaggaagctttgatttttaccgtttcttccctgtattttctttctctactttgaggaaaccaaagtaaccttttgcacctgctctcttgtaatgatatagccagaaaaacgtgttgccttgaaccacttccctcatctctcctccaagacactgtggacttggtcaccagctcctcccttgttctctaagttccactgagctccatgtgccccctctaccatttgcagagtcctgcacagttttctggctggagcctagaacaggcctcccaagttttaggacaaacagctcagttctagtctctctggggccacacagaaactctttttgggctcctttttctccctctggatcaaagtaggcaggaccatgggaccaggtcttggagctgagcctctcacctgtactcttccgaaaaatcctcttcctctgaggctggatcctagccttatcctctgatctccatggcttcctcctccctcctgccgactcctgggttgagctgttgcctcagtcccccaacagatgcttttctgtctctgcctccctcaccctgagccccttccttgctctgcacccccatatggtcatagcccagatcagctcctaacccttatcaccagctgcctcttctgtgggtgacccaggtccttgtttgctgttgatttctttccagaggggttgagcagggatcctggtttcaatgacggttggaaatagaaatttccagagaagagagtattgggtagatattttttctgaatacaaagtgatgtgtttaaatactgcaattaaagtgatactgaaacacatctgttatgtgactctgtcttagctgggtgtgtctgcatgcaagagtgacaccctccattagacctagctagactgtgcagtgatgtggtggggaggaccagccagggaagagggagcacctcagcagacacaggcaccagccaggatgctaaggacctttagccaagtctgccaactattctcctccatggggagaggaaacatccatttccagtggtagaaaggcagacccgaatgtaccagggagcttccaaatggagggtggtatgttgggttcttaggagctgtacccttcatgaacacccttctgagaagaggagcatgctgatcactgctgcaaaatatgcaaaacaaagggaaggggcaatgtcctgtgcaccctttattatcaggccacccccctccccagccccccaggtcagagtagacacagtgaaggactatgtggggactgttgttctagagacctggcagccaactcagggagggggctggtttccaccctcaagattaagacagcagcctaattaaaaaaaaaatctgtaagcatgtacctccccccagcttccaaaacaacccccaccccacccctaccaggccataggaagttggggagggagtgctgaggagctccaggaaacactcccaagtgtgtcgacagtggcagaggcagttggggccaaacaaaggttgattcttccattcttatctccataaagccagacctttcccttcagcactcctccacccccatctccttcttgcttttctccaactcctctaatcataggttcttccctaggacagaggggaggcgaaatgatgaggttcagagtcttccctcaaaggcgatggctgccttgagggttggagcaaaggatgatgagcaaaagacgatggtaatcagtagggaagtccagcccacttgcatctagttgcacatcttgccttgagagtaatccagtgagggtctgtcccagctaggacatcaagtaggaggggtgggttcagggttcagattcctaggaaatatgggaggagaggaaaaggcaacttggatgcacctccagcttcaggcctagcaacctgcaatgcatctcaccctgagtttgctggaatgtgtatgtatgctttgggaggaagggctgtgtgtgtattgcggggtggggtggggcagctggttccctctgacagctggacagcttgccctgaagaatttgcctgctttctggaaaaatccaactttcccaccgtgggcctgagcgtcctggtacagcaatggcgccacctgctggccttattgaggtcctactgctcagcctcagctcaatcgcctccatgttgggcttctctccctggctgccccaccctctagtccaatttctcttgtacacaaagctcatataactatagaacgtcactgttgaagagaactttaaagatacatttaattaaactcccttatggtatagttaaagacaaactaaggctcagagaagggaggtggcttgcccaatcacccagaattccaaagtcctgaatctgtagttttcccttccatcatatcatcctactcttctgccgagtcctccgtgttactccagttggatgtcatgaagccagtgtggcagtgtgaagataggtttgggacttcacttctggagcatttcatcaacataagctatcctaggcctggccagccaagcaggtcctggaggagccccaggacaaagatcacaggaggccatgaggttcggcttcttcggcgcccacagtgagcccaggaaaattagctgtagggtattacactgttgactatggagagcatatctggaattatcttcagccagattttcatctgaatggataaatgggaataccatctaagtccagataaatagatcacttccatctcatcccttctaggtagattaatcccacacttcctcttcacacaaaaccagtaataggtcatcgattttgtgcaacaggatgctgcttctcttcctaaagcccccatcgaagaggcttccagccaccattcaatcattcatcaagtcttatgatgtgccagacactgcgcgaaatgtgccagaacatctgttatgtgccagacactgttcttgagactggggatacagcaaacactcatgaagcttataattctagcagaagaggacagtaaacaatgtcatctcagtaagtatatacatgtgttttcaggattgagagctatgaaaaacataaaatatattgagaataatggttggtattttacatatggtggttacttttagaaaaataacagtggagagcacagcttcacttgaatgaagtggagaagcaggttgtatgccaagctgggagagattatcccacacaggggaaaggacaagtgcaaagccctatgatgaaaagctgccaagtgcagaaagcctcagatggcagggggcaagatggccatgaggttgtgtcagtgagtgggggtggggagaggcaggaggtcagactacatggggcctttttagttgtagattgggaagccactggagggttttgagcagagaagtcatatcatctgctttatgttttaaaaggatcatgctggctgctgagtagagaatagaggttgagggataagaaagtagaaggagaccgtagcaagaagaacgatcatggctgggagcaggtgatcatattggcagtgatgagatcaagcagaattcaaaaagtggtttcaaagtagaggtaacaggacttgctcagtctatttatttcttcaaataataatcatatttacaatgatagtagctaacagtttttgagtgcttactgtatgaaaattgagatatggtgccaatatttaaatagcatattttacttaacattcacagaaaccctgtgaagtaggttctattatctcagaaaaagaaactgaaactcagagaataacaagggactgtgttacgtgcacagtggcagaggcaaagatgaataggatgtgagtttatttgaaccccaaatgtttaaatcttggggataatacaacacacatttaaacaaagaagcaagaaaaaaaatgcacaacagaaagtgagaaataacacgaggaaagactaaatgaagtgctttgtatctagatgtgggcaggaccctttccagctgagaagatctgagactgggtcatgaacaggtggtttctgagtgggtcctgtaaaaatgaatacgattttgatgatagtaatgagtaaggacatttgagactgatagaagagtacatacaatatgtagtgatggggaaagataaggtactgtcaaaggacaatgtgttttctggtatgacagagaagtagaatgtgttaagggaagccgagtaccagaaagatccgggtgtcacagtttgtgtagggtgtttaaagctaaaccacagagtttaattttatccaatagaagaggagccacagaagagtttccatttattcattaatttattcatttattcaaaaaatatttgagtgcttattataagccaggtactatgccaggcacctgggataagacatagtcccttctgtcaagtctttacattgggtggatgtgggagggacagatgacagaacaatatgcattgagtgtaagtgctatggtataggaagctctgagtgggaggggcatggaagccgtggaagaccatggaaggcttcccaggagaagtgacgtctggactgatcctttggtcaagcaggagttaaagaggagaaaaggagagatatgggtgttcccgagagaggaagaagccttgtcccaggagcaaagtgagggtgattgttccagaaatgtgagtgattcttttaaggctcaagcaaagcatgtgattcttctttataccttctatttctttgctgagtgtttctgttcttttgtttcaagcatgctgcaattgctcattaaagcatgtttatgatggctgtctgttttaaaattcttgtcagatggtttcaacatctttatcatctcaatgttggcatctgttaatggttttttctcaatcaaattgagattttcctggttcttggtattaccagtgattttaattgcatctggaaatttgggatttatgttgaaagactggatcttattgaaagattctgtttagcacccctcctttgataccacactggtgggtccaggttccccattcagctgttgacaccttcagggcagagaggtgggatggggtgaagggggtacctcattattgctggcccaggttagaagttcaggcttcccagtagatctctgctgataccaccctggtgccatgtcattccttgagtccaaaagtccctcccaattctgccttcttctctctacatatcggagtctccctatgtttgacttatatataatgtccagggtttttagagttagttaacaggaggcataagaaaaagtgtgtccactccatcttgtctggaactggaagttcaagtcgaatataagagagaggagaggaaattacaagccatgagactggagagttaggcaggttctacaccagctattctcaaagccctcttacactcttaaaaatttagaacttcaaagagcttttgattttgaaagttacatctatcaattattactgtttcaaaaattaaaattgagaaaattttatttattaatttgtttaaaaataacaataattattcaattacatgataatgtaagtaatgcttttcttaatgaaaaataattatattttccaaaacaaaaacaattaggaaaaagagtgtcattgttttagactttggtaaatctctctaatatctggctgaagagaagaatgctgattctttttttttttttttttttttgagacggagtctcgctctgtcacccaggctggagtgtagtggtgtgatctcggctcactgcaagctctgcctcccgggttcacgccattctcctgcctcagcctcccaagtagctgggactacaggcacccgccaccacgcccggctaatttttttgtatttttagtagagatggggtttcaccgtgttagccaggctggtctcgatctcctgacctcatgatccacccacctcagcctcccaaagcgctgggattacaggtgtgagacaccgcgcccagcccccgaatgctgattcttttatctgcttctgtattcaatctgttgtgatatgatgggtagcctctgaaacactccactgtatacttgtgaaagaatgaatgtgaaaaaggaaaatagatttgtagtattattattcaaattgttttgacctcagagaccacttggaaatgttttagggaacccccagaggaccttggatcatgctttgagaaccgcggctctagatatgttactatttcagtagcatctaagtacatgtggctgctgagcacttgtaatgtggctagtgcaaatgagagacaggacttccagctatatgtaatttaataaactcaaatttaaaaactggaacctcataaaatgttttgttgttgttgttaaacatgaccttatagttttggtaggaa

SEQ ID NO: 18 - DNA, 2598 bp, Artificial Sequence
  Synthetic
agtgtcttccactcggtgcgtctctctaggagccgcgcgggaaggatgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggagctggaaccctgaccactcttcaggaagaaaggagtctgcacatgcagctgcaccctccctccgatccttcctcccacctccccctcccccttctcccacccctgcccccacttcctgtttgggcccctctcccatccagtgtctcacagccctgcttaccagataatgctactttatttatacactgtctagggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtgactcggctaggtggggcaaggctgggcagtgtccgagagagcacccctctctgcatctgaccacgtgctacccccatgctggaggtgacatctcttacgcccaacccttccccactgcacacacctcagaggctgttcttggggccctacaccttgaggaggggcaggtaaactcctgtcctttacacattcggctccctggagccagactctggtcttctttgggtaaacgtgtgacgggggaaagccaaggtctggagaagctcccaggaacaatcgatggccttgcagcactcacacaggacccccttcccctaccccctcctctctgccgcaatacaggaacccccaggggaaagatgagcttttctaggctacaattttctcccaggaagctttgatttttaccgtttcttccctgtattttctttctctactttgaggaaaccaaagtaaccttttgcacctgctctcttgtaatgatatagccagaaaaacgtgttgccttgaaccacttccctcatctctcctccaagacactgtggacttggtcaccagctcctcccttgttctctaagttccactgagctccatgtgccccctctaccatttgcagagtcctgcacagttttctggctggagcctagaacaggcctcccaagttttaggacaaacagctcagttctagtctctctggggccacacagaaactctttttgggctcctttttctccctctggatcaaagtaggcaggaccatgggaccaggtcttggagctgagcctctcacctgtactcttccgaaaaatcctcttcctctgaggctggatcctagccttatcctctgatctccatggcttcctcctccctcctgccgactcctgggttgagctgttgcctcagtcccccaacagatgcttttctgtctctgcctccctcaccctgagccccttccttgctctgcacccccatatggtcatagcccagatcagctcctaacccttatcaccagctgcctcttctgtgggtgacccaggtccttgtttgctgttgatttctttccagaggggttgagcagggatcctggtttcaatgacggttggaaatagaaatttccagagaagagagtattgggtagatattttttctgaatacaaagtgatgtgtttaaatactgcaattaaagtgatactgaaacaca

SEQ ID NO: 19 - protein, 107 aa, Artificial Sequence
  Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 20 - protein, 24 aa, Artificial Sequence
  Synthetic
IYIWAPLAGTCGVLLLSLVITLYC

SEQ ID NO: 21 - protein, 68 aa, Artificial Sequence
  Synthetic
FWVLVVVGGVLACYSLLVTVAFIIFWVRSKRSRLLHSDYMNMTPRRPGPTRKHYQPYAPPRDFAAYRS

SEQ ID NO: 22 - protein, 41 aa, Artificial Sequence
  Synthetic
KRGRKKLLYIFKQPFMRVQTTQEEDGCSCRFPEEEEGGCEL

SEQ ID NO: 23 - protein, 113 aa, Artificial Sequence
  Synthetic
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - DNA, 25 bp, Artificial Sequence
  Synthetic
  misc_feature (1)..(23): n is a, c, g, or t
nnnnnnnnnnnnnnnnnnnnnnngg

SEQ ID NO: 25 - DNA, 25 bp, Artificial Sequence
  Synthetic
  misc_feature (3)..(25): n is a, c, g, or t
ccnnnnnnnnnnnnnnnnnnnnnnn

SEQ ID NO: 26 - RNA, 22 nt, Artificial Sequence
  Synthetic
  misc_feature (1)..(22): n is a, c, g, or u
nnnnnnnnnnnnnnnnnnnnnn

SEQ ID NO: 27 - DNA, 25 bp, Artificial Sequence
  Synthetic
  misc_feature (1)..(23): n is a, c, g, or t
nnnnnnnnnnnnnnnnnnnnnnntt

SEQ ID NO: 28 - DNA, 25 bp, Artificial Sequence
  Synthetic
  misc_feature (3)..(25): n is a, c, g, or t
aannnnnnnnnnnnnnnnnnnnnnn

SEQ ID NO: 29 - DNA, 109 bp, Artificial Sequence
  Synthetic
tcccccacccctctcccgggcggagggggcgggaagagcgcgtcctggccaagccgagtagtgtcttccactcggtgcgtctctctaggagccgcgcgggaaggatgct

SEQ ID NO: 30 - DNA, 109 bp, Artificial Sequence
  Synthetic
agggggtggggagagggcccgcctcccccgcccttctcgcgcaggaccggttcggctcatcacagaaggtgagccacgcagagagatcctcggcgcgcccttcctacga

SEQ ID NO: 31 - DNA, 109 bp, Artificial Sequence
  Synthetic
ggtccgcaggggcgcgcgcgcagggcccaggatgccgcggggctggaccgcgctttgcttgctgagtttgctgcgtgagtaccgcccgcgcgccgcggccgcttggctt

SEQ ID NO: 32 - DNA, 109 bp, Artificial Sequence
  Synthetic
ccaggcgtccccgcgcgcgcgtcccgggtcctacggcgccccgacctggcgcgaaacgaacgactcaaacgacgcactcatggcgggcgcgcggcgccggcgaaccgaa

SEQ ID NO: 33 - protein, 24 aa, Artificial Sequence
  Synthetic
VRRGARAGPRMPRGWTALCLLSLL

SEQ ID NO: 34 - DNA, 109 bp, Artificial Sequence
  Synthetic
cgtctcacggagggaagggacccctgcccaacccacgcactgccgcccacagctgcttccccccggggccagcgcctcacctgggagctgacgggggtgggaggggaag

SEQ ID NO: 35 - DNA, 109 bp, Artificial Sequence
  Synthetic
gcagagtgcctcccttccctggggacgggttgggtgcgtgacggcgggtgtcgacgaaggggggccccggtcgcggagtggaccctcgactgcccccaccctccccttc

SEQ ID NO: 36 - DNA, 109 bp, Artificial Sequence
  Synthetic
ctgccaacaaggggcagcagggaatgttctagacgaattctacctgaagtcaggaaattgcatcaggtgatatcaccaggctgatatctttaggttctgattcttcctc

SEQ ID NO: 37 - DNA, 109 bp, Artificial Sequence
  Synthetic
gacggttgttccccgtcgtcccttacaagatctgcttaagatggacttcagtcctttaacgtagtccactatagtggtccgactatagaaatccaagactaagaaggag

SEQ ID NO: 38 - DNA, 109 bp, Artificial Sequence
  Synthetic
ctcgtttccctatagcttctgggttcatgagtcttgacaacaacggtactgctaccccagagttacctacccagggaacattttcaaatgtttctacaaatgtatccta

SEQ ID NO: 39 - DNA, 109 bp, Artificial Sequence
  Synthetic
gagcaaagggatatcgaagacccaagtactcagaactgttgttgccatgacgatggggtctcaatggatgggtcccttgtaaaagtttacaaagatgtttacataggat

SEQ ID NO: 40 - protein, 32 aa, Artificial Sequence
  Synthetic
PSGFMSLDNNGTATPELPTQGTFSNVSTNVSY

SEQ ID NO: 41 - DNA, 109 bp, Artificial Sequence
  Synthetic
ccaagaaactacaacacctagtacccttggaagtaccagcctgcaccctgtgtctcaacatggcaatgaggccacaacaaacatcacaggtaaaaacagcatttgtgtc

SEQ ID NO: 42 - DNA, 109 bp, Artificial Sequence
  Synthetic
ggttctttgatgttgtggatcatgggaaccttcatggtcggacgtgggacacagagttgtaccgttactccggtgttgtttgtagtgtccatttttgtcgtaaacacag

SEQ ID NO: 43 - protein, 29 aa, Artificial Sequence
  Synthetic
QETTTPSTLGSTSLHPVSQHGNEATTNIT

SEQ ID NO: 44 - DNA, 109 bp, Artificial Sequence
  Synthetic
agatcccggagagatgctggtgatgcttgggtaaagcatttaggatgttttcagaccgctcccctctccacagaggaaattatacaagtccctagtattaatgacttga

SEQ ID NO: 45 - DNA, 109 bp, Artificial Sequence
  Synthetic
tctagggcctctctacgaccactacgaacccatttcgtaaatcctacaaaagtctggcgaggggagaggtgtctcctttaatatgttcagggatcataattactgaact

SEQ ID NO: 46 - DNA, 109 bp, Artificial Sequence
  Synthetic
ccccagaaaccgaagagattccatctggtcgtagataaagtcaaagtcctgtggagattatgtaagatctaagatcgagaacaaggttgtcgaactttactcaaaccag

SEQ ID NO: 47 - DNA, 109 bp, Artificial Sequence
  Synthetic
agggatgggacacgaagtaactgttaactccccaaactcccttttctgtgttgaagtgcatgcccatatcatgacctgagattttgtgtatctatgagcacatgcacac

SEQ ID NO: 48 - DNA, 109 bp, Artificial Sequence
  Synthetic
tccctaccctgtgcttcattgacaattgaggggtttgagggaaaagacacaacttcacgtacgggtatagtactggactctaaaacacatagatactcgtgtacgtgtg

SEQ ID NO: 49 - DNA, 109 bp, Artificial Sequence
  Synthetic
atgcacacccatgttttggtctcttccagaaacgacagtcaaattcacatctacctctgtgataacctcagtttatggaaacacaaactcttctgtccagtcacagacc

SEQ ID NO: 50 - DNA, 109 bp, Artificial Sequence
  Synthetic
tacgtgtgggtacaaaaccagagaaggtctttgctgtcagtttaagtgtagatggagacactattggagtcaaatacctttgtgtttgagaagacaggtcagtgtctgg

SEQ ID NO: 51 - protein, 27 aa, Artificial Sequence
  Synthetic
ETTVKFTSTSVITSVYGNTNSSVQSQT

SEQ ID NO: 52 - DNA, 109 bp, Artificial Sequence
  Synthetic
tctgtaatcagcacagtgttcaccaccccagccaacgtttcaactccagagacaaccttgaagcctagcctgtcacctggaaatgtttcagacctttcaaccactagca

SEQ ID NO: 53 - DNA, 109 bp, Artificial Sequence
  Synthetic
agacattagtcgtgtcacaagtggtggggtcggttgcaaagttgaggtctctgttggaacttcggatcggacagtggacctttacaaagtctggaaagttggtgatcgt

SEQ ID NO: 54 - protein, 36 aa, Artificial Sequence
  Synthetic
SVISTVFTTPANVSTPETTLKPSLSPGNVSDLSTTS

SEQ ID NO: 55 - DNA, 109 bp, Artificial Sequence
  Synthetic
ctagccttgcaacatctcccactaaaccctatacatcatcttctcctatcctaagtgacatcaaggtgggtgaattgggccaaaaatggcagattgcccctcacttcat

SEQ ID NO: 56 - DNA, 109 bp, Artificial Sequence
  Synthetic
gatcggaacgttgtagagggtgatttgggatatgtagtagaagaggataggattcactgtagttccacccacttaacccggtttttaccgtctaacggggagtgaagta

SEQ ID NO: 57 - protein, 22 aa, Artificial Sequence
  Synthetic
TSLATSPTKPYTSSSPILSDIK

SEQ ID NO: 58 - DNA, 28 bp, Artificial Sequence
  Synthetic
caaactcagcaagcaaagcgcggtccag

SEQ ID NO: 59 - DNA, 29 bp, Artificial Sequence
  Synthetic
  misc_feature (18)..(18): n is a, c, g, or t
caaactcagcaagcaaangcgcggtccag

SEQ ID NO: 60 - DNA, 28 bp, Artificial Sequence
  Synthetic
gtgcaggctggtacttccaagggtacta

SEQ ID NO: 61 - DNA, 27 bp, Artificial Sequence
  Synthetic
gtgcaggctggtacttcaagggtacta

SEQ ID NO: 62 - DNA, 16 bp, Artificial Sequence
  Synthetic
gtgcacaagggtacta

SEQ ID NO: 63 - DNA, 20 bp, Artificial Sequence
  Synthetic
caaactcagcaagcaaagcg

SEQ ID NO: 64 - DNA, 20 bp, Artificial Sequence
  Synthetic
gtgcaggctggtagtaccaa

(19) United States
(12) Patent Application Publication
(10) Pub. No.: US 2024/0366675 A1
(43) Pub. Date: Nov. 7, 2024

(54) COMPOSITIONS AND METHODS FOR CD34 GENE MODIFICATION

(71) Applicant: VOR BIOPHARMA INC., Cambridge, MA (US)

(72) Inventor: Elizabeth Paik, Cambridge, MA (US)

(73) Assignee: VOR BIOPHARMA INC., Cambridge, MA (US)

(21) Appl. No.: 18/269,633

(22) PCT Filed: Dec. 31, 2021
(86) PCT No.: PCT/US2021/065813

§ 371 Date: Jun. 26, 2023

Related U.S. Application Data
(60) Provisional application No. 63/132,852, filed on Dec. 31, 2020.

(51) Int. Cl. A61K 35/28; C07K 14/705; C12N 5/0789; C12N 9/22; C12N 15/11; C12N 15/90

(52) U.S. Cl. CPC A61K 35/28; C07K 14/70596; C12N 5/0647; C12N 9/22; C12N 15/11; C12N 15/907; C12N 2310/20; C12N 2310/313; C12N 2310/321; C12N 2510/00

(57) ABSTRACT

Provided herein are gRNA comprising a targeting domain that targets CD34, which may be used, for example, to make modifications in cells. Also provided herein are methods of genetically engineered cell having a modification (e.g., insertion or deletion) in the CD34 gene and methods involving administering such genetically engineered cells to a subject, such as a subject having a hematopoietic malignancy.

### RELATED APPLICATIONS

This application claims the benefit under 35 U.S.C. 119(e) of U.S. provisional application No. 63/132,852, filed Dec. 31, 2020, which is incorporated by reference herein in its entirety.

### BACKGROUND

When a subject is administered an immunotherapy targeting an antigen associated with a disease or condition, e.g., an anti-cancer CAR-T therapy, the therapy can deplete not only the pathological cells intended to be targeted, but also non-pathological cells that may express the targeted antigen. This “on-target, off-disease” effect has been reported for some CAR-T therapeutics, e.g., those targeting CD19 or CD33. If the targeted antigen is expressed on the surface of cells required for survival or the subject, or on the surface of cells the depletion of which is of significant detriment to the health of the subject, the subject may not be able to receive the immunotherapy, or may have to face severe side effects once administered such a therapy.

### SUMMARY

Aspects of the present disclosure describe compositions, methods, strategies, and treatment modalities that address the detrimental on-target, off-disease effects of certain immunotherapeutic approaches, e.g., of immunotherapeutics comprising lymphocyte effector cells targeting a specific antigen in a subject in need thereof, such a s CAR-T cells or CAR-NK cells. The disclosure is directed, in part, to compositions, methods, strategies, and treatment modalities that reduce (e.g., deplete) a population of stem cells (e.g., CD34-expressing hematopoietic stem cells (HSCs)) in a subject using an immunotherapeutic approach, e.g., comprising lymphocyte effector cells targeting CD34, such as CAR-T cells or CAR-NK cells. In some aspects, such compositions, methods, strategies, and treatment modalities replace or replenish a population of cells (e.g., hematopoietic cells, e.g., hematopoietic stem cells (HSCs)) with genetically engineered cells (e.g., genetically engineered stem cells, e.g., HSCs) that do not express CD34, express a reduced level of CD34 (e.g., relative at a wild type cell), or express a variant form of CD34 that is not recognized by an immunotherapeutic agent targeting CD34. Without wishing to be bound by theory, the genetically engineered cells (e.g., genetically engineered stem cells, e.g., HSCs) that do not express CD34, express a reduced level of CD34 (e.g., relative at a wild type cell), or express a variant form of CD34 that is not recognized by an immunotherapeutic agent targeting CD34 are thought to evade killing by immunotherapeutic approaches targeting CD34, thus providing a subject in need thereof with a healthy cell population (e.g., stem cell population) to replace a targeted (e.g., malignant) cell population. In some embodiments, the immunotherapeutic approach, e.g., comprising lymphocyte effector cells targeting CD34, such as CAR-T cells or CAR-NK cells, and the genetically engineered cells (e.g., genetically engineered stem cells, e.g., HSCs) that do not express CD34, express a reduced level of CD34 (e.g., relative at a wild type cell), or express a variant form of CD34 that is not recognized by an immunotherapeutic agent targeting CD34 are administered in combination to a subject in need thereof.

Accordingly, aspects of the present disclosure provide genetically engineered hematopoietic cells, or descendants thereof, comprising a modified gene encoding CD34. In some embodiments, the modified gene encoding CD34 comprises an INDEL mutation. In some embodiments, the modified gene encoding CD34 is modified such that an exon is skipped. In some embodiments, exon 1, exon 2, or exon 3 of the modified gene encoding CD34 is genetically engineered. In some embodiments, the modified gene encoding CD34 comprises an insertion or deletion immediately proximal to a site cut by an RNA-guided nuclease when bound to a gRNA comprising a targeting domain as provided by any of SEQ ID NO: 11-15. In some embodiments, the modified gene encoding CD34 comprises an insertion or deletion generated by a non-homologous end joining (NHEJ) event or by a homology-directed repair (HDR) event.

In some embodiments, modification of the gene encoding CD34 alters expression of CD34 and/or changes the properties of the hematopoietic cell or a descendent thereof. In some embodiments, expression of CD34 is reduced or eliminated relative to a wild-type counterpart cell that does not harbor the modified gene encoding CD34. In some embodiments, expression of CD34 is less than 25%, less than 20% less than 10% less than 5% less than 2% less than 1%, less than 0.1%, less than 0.01%, or less than 0.001% as compared to the expression level of CD34 in the wild-type counterpart cell that does not harbor a modified gene encoding CD34. In some embodiments, the hematopoietic cell, or descendant thereof has reduced or no binding to an agent comprising an anti-CD34 binding domain. In some embodiments, the hematopoietic cell, or descendant thereof retains the capacity to differentiate normally compared to a population of hematopoietic cells that are not genetically engineered. In some embodiments, the modified gene encoding CD34 results in a loss of function of CD34 in the genetically engineered hematopoietic cell, or descendant thereof.

In some embodiments, the genetically engineered hematopoietic cell or descendent thereof further comprises a modified gene encoding a lineage-specific cell-surface antigen. In some embodiments, the lineage-specific cell-surface antigen is a lymphoid-specific cell-surface antigen. In some embodiments, the lineage-specific cell-surface antigen is a myeloid-specific cell-surface antigen. In some embodiments, the lineage-specific cell-surface antigen is a hematopoietic stem cell-specific cell-surface antigen. In some embodiments, the lineage-specific cell-surface antigen is a hematopoietic stem or progenitor-specific cell-surface antigen. In some embodiments, the lineage-specific cell-surface antigen is a T cell-specific cell-surface antigen. In some embodiments, the lineage-specific cell-surface antigen is a B cell-specific cell-surface antigen. In some embodiments, the lineage-specific cell-surface antigen is an NK cell-specific cell-surface antigen. In some embodiments, the lineage-specific cell-surface antigen is a basophil-specific cell-surface antigen. In some embodiments, the lineage-specific cell-surface antigen is an eosinophil-specific cell-surface antigen. In some embodiments, the lineage-specific cell-surface antigen is a neutrophil-specific cell-surface antigen. In some embodiments, the lineage-specific cell-surface antigen is a monocyte-specific cell-surface antigen. In some embodiments, the lineage-specific cell-surface antigen is an erythrocyte-specific cell-surface antigen. In some embodiments, the lineage-specific cell-surface antigen is CD33, CD123, CLL-1, CD19, CD30, CD5, CD6, CD7, CD38, or BCMA. In some embodiments, the lineage-specific cell surface antigen is expressed on the surface of malignant cells. In some embodiments, the genetically engineered hematopoietic cell, or descendant thereof has reduced or no binding to an agent comprising an binding domain that targets the lineage-specific cell-surface antigen. In some embodiments, the hematopoietic cell, or descendant thereof, lacks a CD34 epitope or has a modified CD34 epitope.

Aspects of the present disclosure provide guide RNAs (gRNA) comprising a targeting domain comprising a sequence described in Tables 1 and 2. In some aspects, the gRNA comprises a targeting domain, wherein the targeting domain comprises a sequence of any one of SEQ ID NOs: 1-15. In some embodiments, the gRNA comprises a first complementarity domain, a linking domain, a second complementarity domain which is complementary to the first complementarity domain, and a proximal domain. In some embodiments, the gRNA is a single guide RNA (sgRNA).

In some embodiments, the gRNA comprises one or more nucleotide residues that are chemically modified. In some embodiments, the gRNA comprises one or more nucleotide residues that comprise a 2′O-methyl moiety. In some embodiments, the gRNA comprises one or more nucleotide residues that comprise a phosphorothioate. In some embodiments, the gRNA comprises one or more nucleotide residues that comprise a thioPACE moiety.

Aspects of the present disclosure provide methods of producing a genetically engineered cell, comprising: providing a cell, and contacting the cell with (i) any of the gRNAs described herein; and (ii) an RNA-guided nuclease that binds the gRNA, thus forming a ribonucleoprotein (RNP) complex under conditions suitable for the gRNA of (i) to form and/or maintain an RNP complex with the RNA-guided nuclease of (ii) and for the RNP complex to bind a target domain in the genome of the cell. In some embodiments, the contacting comprises introducing (i) and (ii) into the cell in the form of a pre-formed ribonucleoprotein (RNP) complex. In some embodiments, the contacting comprises introducing (i) and/or (ii) into the cell in the form of a nucleic acid encoding the gRNA of (i) and/or the RNA-guided nuclease of (ii). In some embodiments, the nucleic acid encoding the gRNA of (i) and/or the RNA-guided nuclease of (ii) is an RNA, preferably an mRNA or an mRNA analog. In some embodiments, the ribonucleoprotein complex is introduced into the cell via electroporation.

In some embodiments, the RNA-guided nuclease is a CRISPR/Cas nuclease. In some embodiments, the CRISPR/Cas nuclease is a Cas9 nuclease. In some embodiments, the CRISPR/Cas nuclease is an spCas nuclease. In some embodiments, the Cas nuclease in an saCas nuclease. In some embodiments, the CRISPR/Cas nuclease is a Cpf1 nuclease.

In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte.

Aspects of the present disclosure provide genetically engineered cells obtained by any of the methods described herein. Aspects of the present disclosure provide cell populations comprising the genetically engineered cells described herein.

Aspects of the present disclosure provide cell populations comprising a genetically engineered cell, wherein the genetically engineered cell comprises a genomic modification that consists of an insertion or deletion immediately proximal to a site cut by an RNA-guided nuclease when bound to a gRNA comprising a targeting domain as described in any of Tables 1 and 2. In some embodiments, wherein the genomic modification is an insertion or deletion generated by a non-homologous end joining (NHEJ) event. In some embodiments, wherein the genomic modification is an insertion or deletion generated by a homology-directed repair (HDR) event. In some embodiments, the genomic modification results in a loss-of function of CD34 in a genetically engineered cell harboring such a genomic modification. In some embodiments, the genomic modification results in a reduction of expression of CD34 to less than 25%, less than 20% less than 10% less than 5% less than 2% less than 1%, less than 0.1%, less than 0.01%, or less than 0.001% as compared to the expression level of CD34 in wild-type cells of the same cell type that do not harbor a genomic modification of CD34. In some embodiments, the genetically engineered cell is a hematopoietic stem or progenitor cell. In some embodiments, the genetically engineered cell is an immune effector cell. In some embodiments, the genetically engineered cell is a T-lymphocyte. In some embodiments, the immune effector cell expresses a chimeric antigen receptor (CAR). In some embodiments, the CAR targets CD34.

Aspects of the present disclosure provide methods comprising administering to a subject in need thereof any of the genetically engineered cells described herein (e.g., a genetically engineered hematopoietic cell or descendent thereof described herein) or any of the cell populations described herein. In some embodiments, the subject has or has been diagnosed with a hematopoietic malignancy. In some embodiments, the method further comprises administering to the subject an effective amount of an agent that targets CD34, wherein the agent comprises an antigen-binding fragment that binds CD34. In some embodiments, the agent is an antibody-drug conjugate or an immune effector cell expressing a chimeric antigen receptor (CAR). In some embodiments, the subject has a malignancy associated or characterized by the expression of CD34 on malignant cells. In some embodiments, the malignant cells are cancer stem cells. In some embodiments, the subject has a hematopoietic malignancy. In some embodiments, the subject has an autoimmune disease.

Aspects of the present disclosure provide pharmaceutical compositions comprising a genetically engineered hematopoietic cell described herein (e.g., a genetically engineered HSC described herein). In some embodiments, the pharmaceutical composition further comprises an effective amount of an agent that targets CD34, e.g., an antigen-binding fragment that binds CD34, an antibody-drug conjugate or an immune effector cell expressing a chimeric antigen receptor (CAR).

Aspects of the present disclosure provide genetically engineered hematopoietic stem cells comprising a modification in a gene encoding CD34. In some embodiments, the genetically engineered hematopoietic stem cell does not express a naturally-occurring CD34 protein. In some embodiments, the genetically engineered hematopoietic stem cell is functionally indistinguishable from a naturally occurring hematopoietic stem cell expressing CD34. In some embodiments, the genetically engineered hematopoietic stem cell expresses one or more hematopoietic stem cell markers. In some embodiments, the genetically engineered hematopoietic stem cell expresses one or more of CD49c, CD71, CD90, CD117, CD135, CD201, CD228, CD243, CD292, CDw293, CD309, CD318, CD325, and CD349. In some embodiments, the genetically engineered hematopoietic stem cell does not express CD34 on its cell surface. In some embodiments, the genetically engineered hematopoietic stem cell does not express a CD34 epitope recognized by an anti-CD34 antibody on its cell surface. In some embodiments, the genetically engineered hematopoietic stem cell does not express CD34 on its cell surface. In some embodiments, the genetically engineered hematopoietic stem cell does not express CD34 on its cell surface and expresses one or more of CD71, CD90, CD201, and CD49c. In some embodiments, the genetically engineered hematopoietic stem cell does not express CD34 on its cell surface and expresses one or more of CD90, CD201, and CD49c. In some embodiments, the genetically engineered hematopoietic stem cell does not express a lineage-specific surface marker characteristic of differentiated hematopoietic cells (lin−). In some embodiments, the genetically engineered hematopoietic stem cell does not express CD2, CD3, CD4, CD8, CD11b, CD14, CD15, CD16, CD19, CD56, CD123, or CD235a, or any combination thereof. In some embodiments, the genetically engineered hematopoietic stem cell is capable of long-term engraftment into a human recipient. In some embodiments, the genetically engineered hematopoietic stem cell is capable of reconstituting the hematopoietic system in a human recipient after engraftment. In some embodiments, the genetically engineered hematopoietic stem cell does not express CD34 on its cell surface and expresses CD90, CD201, and CD49c. In some embodiments, the genetically engineered hematopoietic stem cell does not express CD34 on its cell surface, expresses one or more of CD90, CD201, and CD49c, and is negative for CD2, CD3, CD4, CD8, CD11b, CD14, CD15, CD16, CD19, CD45RA, CD56, CD123, and CD235a. In some embodiments, the genetically engineered hematopoietic stem cell does not express CD34 on its cell surface, expresses CD90, CD201, and CD49c, and is negative for CD2, CD3, CD4, CD8, CD11b, CD14, CD15, CD16, CD19, CD45RA, CD56, CD123, and CD235a.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 is a schematic of the CD34 gene showing the coding region of two splice variants of human CD34 (“CD34 CDS”). A number of exemplary Cas9 gRNA target sequences and their respective binding site within the CD34 gene, are indicated by short arrows in the lower part of the panel, and the location within the named gRNAs CD34-1 through CD34-5 are indicated by short, dark arrows in the middle of the panel.

FIGS. 2A-2C present schematics showing the location of exemplary guide RNAs described herein relative to the CD34 gene. FIG. 2A is a schematic showing the location of the exemplary guide RNAs described herein relative to the nucleic acid sequence encoding exon 1 of CD34. FIG. 2B is a schematic showing the location of exemplary guide RNAs described herein relative to the nucleic acid sequence encoding exon 2 of CD34. FIG. 2C is a schematic showing the location of an exemplary guide RNA described herein relative to the nucleic acid sequence encoding exon 3 of CD34.

FIGS. 3A-3D show survival and editing efficiency of CD34-edited hematopoietic stem cells. FIG. 3A is a schematic showing the workflow of the experiment. CD34+ cells were thawed and electroporated with Cas9 protein and the exemplary CD34-targeting gRNAs described herein, or a control gRNA targeting CD5 (“Ctrl”), followed by analysis of cell viability and editing efficiency by sequencing. FIG. 3B shows average cell count as measured 24 and 48 hours post electroporation with the indicated sgRNA conditions. FIG. 3C shows cell viability as measured 24 and 48 hours post electroporation with the indicated sgRNA conditions. FIG. 3D shows editing efficiency of the CD34 locus as determined by sequencing analysis.

FIG. 4 shows an INDEL (insertion/deletion) distribution for HSC edited with the exemplary CD34-targeting gRNA referred to as guide CD34-2. The X-axis indicates the size of the INDEL and the Y-axis indicates the percentage of the specific INDEL in the mixture.

FIGS. 5A-5C show survival and editing efficiency of CD34-edited donor hematopoietic stem cells. FIG. 5A is a schematic showing the workflow of the experiment. CD34+ cells were thawed and electroporated with Cas9 protein and the exemplary CD34-targeting gRNAs described herein followed by analysis of cell viability and editing efficiency by sequencing. FIG. 5B shows average cell count as measured 24 and 96 hours post electroporation with the indicated gRNA conditions. FIG. 5C shows cell viability as measured 24 and 96 hours post electroporation with the indicated gRNA conditions.

FIGS. 6A and 6B show knockdown of expression of CD34 in CD34-edited donor hematopoietic stem cells at 96 hours post electroporation following the workflow presented in FIG. 5A. FIG. 6A presents plots showing the expression of CD34 in donor hematopoietic stem cells edited using the indicated exemplary CD34-targeting gRNAs, mock edited cells (“Mock”), and cells that were not electroporated (“No EP”) by flow cytometric analysis using an anti-CD34-PE antibody. The X-axis indicates the intensity of antibody staining (anti-CD34-PE Texas) and the Y-axis corresponds to the number of cells. FIG. 6B presents a plot showing expression of CD34 in donor hematopoietic stem cells edited using the indicated exemplary CD34-targeting gRNAs, mock edited cells (“Mock”), and cells that were not electroporated (“No EP”) based on the relative intensity of anti-CD34-PE antibody by flow cytometric analysis.

FIGS. 7A-7D present flow cytometric analysis plots of CD34-edited donor hematopoietic stem cells at 96 hours post electroporation. FIG. 7A shows an unedited control population of cells. FIG. 7B shows donor hematopoietic stem cell edited using exemplary CD34-targeting gRNA guide CD34-2. FIG. 7C shows donor hematopoietic stem cell edited using exemplary CD34-targeting gRNA guide CD34-3. FIG. 7D shows donor hematopoietic stem cell edited using exemplary CD34-targeting gRNA guide CD34-5. The X-axis shows expression of CD34 based on binding of an anti-CD34 antibody (anti-CD34(PE Texas)), and the Y-axis shows live/dead staining (L/D (APC Cy7)).

FIG. 8 is a schematic showing the workflow of a large scale editing experiment targeting CD34 in HSCs and measuring the effect of editing at 48 hrs and 168 hrs post-editing. CD34+ cells were thawed and electroporated with Cas9 protein and the exemplary CD34-targeting gRNAs described herein followed by harvesting of cells at 48 hrs and 168 hrs following electroporation for analysis. Media was changed after the 48 hr and 120 hr time points.

FIGS. 9A and 9B show knockdown of expression of CD34 in CD34-edited donor hematopoietic stem cells at 48 hours post electroporation following the workflow presented in FIG. 8. FIG. 9A presents plots showing the expression of CD34 in donor hematopoietic stem cells edited using the indicated exemplary CD34-targeting gRNAs, mock edited cells (“Mock”), and cells that were not electroporated (“No EP”) by flow cytometric analysis using an anti-CD34-PE antibody. The X-axis indicates the intensity of antibody staining (anti-CD34-PE Texas) and the Y-axis corresponds to the number of cells. FIG. 9B presents a plot showing the percent CD34+ cells in donor hematopoietic stem cells edited using the indicated exemplary CD34-targeting gRNAs, mock edited cells (“Mock”), and cells that were not electroporated (“No EP”) based on the relative intensity of anti-CD34-PE antibody by flow cytometric analysis.

FIGS. 10A and 10B show knockdown of expression of CD34 in CD34-edited donor hematopoietic stem cells at 168 hours post electroporation following the workflow presented in FIG. 8. FIG. 10A presents plots showing the expression of CD34 in donor hematopoietic stem cells edited using the indicated exemplary CD34-targeting gRNAs, mock edited cells (“Mock”), and cells that were not electroporated (“No EP”) by flow cytometric analysis using an anti-CD34-PE antibody. The X-axis indicates the intensity of antibody staining (anti-CD34-PE Texas) and the Y-axis corresponds to the number of cells. FIG. 10B presents a plot showing the percent CD34+ cells in donor hematopoietic stem cells edited using the indicated exemplary CD34-targeting gRNAs, mock edited cells (“Mock”), and cells that were not electroporated (“No EP”) based on the relative intensity of anti-CD34-PE antibody by flow cytometric analysis.

FIGS. 11A-11Q show charts, graphs, and tables showing colony forming unit (CFU) assays for cell lineage differentiation and colony genotyping, as well as editing frequency assays showing INDEL size, position, and allele analysis for exemplary guide RNAs. CFU-GEMM: colony forming unit—granulocyte, erythrocyte, macrophage, megakaryocyte; CFU-GM: colony forming unit—granulocyte, macrophage; CFU-E: Colony forming unit—erythroid; BFU-E: burst forming unit—erythroid. FIG. 11A presents an outline of the experimental approach for CFU assays in CD34-edited HSCs. FIGS. 11B-11C present CFU assay data of CD34-edited HSCs wherein two concentrations of edited cells are plated and used for CFU analysis following editing using either a CRISPR/Cas system comprising either of two exemplary CD34-targeting guide RNAs: CD34-2 (also referred to herein as Guide 2, g2, or sgCD34_2) or CD34-3 (also referred to herein as Guide 3, g3, or sgCD34_3). FIGS. 11D and 11E present editing efficiency data in various HSC burst-forming units (BFUs) and colony forming units (CFUs) engineered using CD34-2 and CD34-3, respectively. FIGS. 11F and 11G present insertion/deletion (INDEL) position distribution for CD34-2- and CD34-3-edited HSC colonies, respectively. FIGS. 11H-11K show allele analyses of CD34-editing of HSCs using CD34-2 or CD34-3, respectively.

FIGS. 11L and 11M show INDEL distribution in analyzed samples of CD34-2- and CD34-3-edited HSC colonies in heat map form, respectively. FIGS. 11N and 11O show a bar graph representation of the INDEL distribution for CD34-edited HSCs edited with CD34-2 and CD34-3, respectively. FIGS. 11P and 11Q shows nucleic acid sequence changes. amino acid sequence changes, frequency, and the protein consequences of dominant INDEL species in HSCs edited with CD34-2 and CD34-3 corresponding to the distribution data shown in FIGS. 11N and 11O, respectively.

FIGS. 12A-12W show graphs of CD34-editing time course data in samples of HSCs from three different donors. FIGS. 12A-12C show viability data across the 0, 24, 48, 120, 168, and 216 hours after electroporation for the three donor populations of CD34 HSCs (Donor 1, Donor 2, and Donor 3, respectively). FIGS. 12D-12I show TIDE analysis of editing efficiency in HSCs at various time points after electroporation as represented by % INDELs for dominant species of INDELs relative to control donor HSC samples that either did not undergo electroporation or were electroporated with control (CTRL) gRNA; FIGS. 12D-12E show data from editing of Donor 1 HSCs. FIGS. 12F-12G shows data from editing of Donor 2 HSCs, and FIGS. 12H-12I show data from editing of Donor 3 HSCs. FIGS. 12J-12V show graphs of flow cytometry analysis of surface expression of CD34 protein in edited HSCs at the indicated time points; FIGS. 12J-12N shows data from editing of Donor 1 HSCs. FIGS. 12O-12S show data from editing of Donor 2 HSCs, and FIGS. 12T-12V show data from editing of Donor 3 HSCs. FIG. 12W shows a graph averaging percent CD34+ cells over time for two donor HSC samples (Donor 1 and Donor 2) edited using exemplary guides CD34-2 or CD34-3; error bars represent standard deviation.

FIGS. 13A-13L show graphs evaluating myeloid in vitro differentiation (IVD) of CD34-edited human hematopoietic stem cells (HSCs). FIG. 13A shows the experimental approach taken for characterizing IVD patterns of CD34-edited HSCs. FIG. 13B shows gel electrophoresis analysis of PCR-amplified genomic DNA isolated at 48 hours post-electroporation from donor Donor 2 HSCs edited using CD34-2 or CD34-3 and prior to IVD. FIGS. 13C and 13D show TIDE analysis of % INDELs in donor Donor 2 HSCs edited using CD34-2 and CD34-3 at 48 hours post-electroporation and prior to IVD. respectively. FIGS. 13E-13F shows flow cytometry analysis of surface expression of CD34 protein in edited HSCs at 0, 2, or 9 days post-electroporation in IVD conditions, measured in CD34 geometric mean fluorescence intensity (FIG. 13E) and % CD34 positive cells (FIG. 13F) in the live population. FIGS. 13G-13I show flow cytometry analysis of surface expression of cell differentiation markers (monocytic markers CD11b, CD14, granulocytic marker CD15, respectively) in edited HSCs at 0, 2, or 9 days post-electroporation in IVD conditions represented as % surface marker-positive cells. FIGS. 13J-13L shows flow cytometry analysis of surface expression of cell differentiation markers in edited HSCs at 0, 2, or 9 days post-electroporation in IVD conditions represented as surface marker geometric mean fluorescence intensity.

FIGS. 14A-14B show in vivo characterization of CD34-edited mouse models. FIG. 14A shows an experimental approach for in vivo characterization of CD34-edited HSCs in an NGS mouse model. Readouts at the different time points include, for example, immunophenotyping of obtained cells. FIG. 14B show exemplary observations characterizing CD34-edited HSCs in the NGS mouse model following engraftment of edited cells into the blood and the bone marrow of the mice.

### DETAILED DESCRIPTION

Some aspects of this disclosure provide compositions, methods, strategies, and treatment modalities related to genetically modified cells, e.g., hematopoietic cells, that are deficient in the expression of an antigen targeted by a therapeutic agent, e.g., an immunotherapeutic agent. The genetically modified cells provided herein are useful, for example, to mitigate, or avoid altogether, certain undesired effects, for example, any on-target, off-disease cytotoxicity, associated with certain immunotherapeutic agents.

Such undesired effects associated with certain immunotherapeutic agents may occur, for example, when healthy cells within a subject in need of an immunotherapeutic intervention express an antigen targeted by an immunotherapeutic agent. For example, a subject may be diagnosed with a malignancy associated with an elevated level of expression of a specific antigen, which is not typically expressed in healthy cells, but may be expressed at relatively low levels in a subset of non-malignant cells within the subject. Alternatively or in addition, a subject may be in need of ablation of cells expressing a specific antigen, such as CD34. Administration of an immunotherapeutic agent, e.g., a CAR-T cell therapeutic or a therapeutic antibody or antibody-drug-conjugate (ADC) targeting the antigen, to the subject may result in efficient killing of the target cells, but may also result in ablation of non-target cells expressing the antigen in the subject. This on-target, off-disease cytotoxicity can result in significant side effects and, in some cases, abrogate the use of an immunotherapeutic agent altogether.

The compositions, methods, strategies, and treatment modalities provided herein address the problem of on-target, off-disease cytotoxicity of certain immunotherapeutic agents. The compositions, methods, strategies, and treatment modalities provided herein also provide an alternative to conventional methods of ablating cell populations expressing CD34, such as irradiation. For example, some aspects of this disclosure provide genetically engineered cells harboring a modification in their genome that results in a lack of expression of an antigen, or a specific form of that antigen, targeted by an immunotherapeutic agent. Such genetically engineered cells, and their progeny, are not targeted by the immunotherapeutic agent, and thus not subject to any cytotoxicity effected by the immunotherapeutic agent. Such cells can be administered to a subject receiving an immunotherapeutic agent targeting the antigen, e.g., in order to replace healthy cells that may have been targeted and killed by the cytotherapeutic agent, and/or in order to provide a population of cells that is resistant to targeting by the cytotherapeutic agent. For example, if healthy hematopoietic cells in the subject express the antigen, genetically engineered hematopoietic cells provided herein, e.g., genetically engineered hematopoietic stem or progenitor cells, may be administered to the subject that do not express the antigen, and thus are not targeted by the cytotherapeutic agent. Such hematopoietic stem or progenitor cells are able to re-populate the hematopoietic niche in the subject and their progeny can reconstitute the various hematopoietic lineages, including any that may have been ablated by the cytotherapeutic agent.

CD34 is a 115 kDa transmembrane glycoprotein receptor rich with O- and N-glycans that is typically expressed on the surface of human hematopoietic stem progenitor and endothelial cells. The function of CD34 is not well-characterized, but expression of Cd34 is thought to be modulated by growth factors such as TGF-β1 and TNF-a and by oxygen concentration. It has been reported that CD34 may modulate cell adhesion, cell shape, or be involved in cell migration (e.g., T cell migration). See Tascv et al. Angiogenesis (2016) 19:325-338. In some embodiments, HSCs express CD34. In some embodiments, CD34 is used as a cell surface marker for detection and/or isolation of stem or progenitor cells (e.g., HSCs).-. The gene encoding human CD34 contains 8 exons and is located on chromosome 1.

In addition to its expression on HSCs, CD34 expression has also been associated with some hematopoietic malignancies.

Due to the shared expression of CD34 on both normal, healthy HSCs as well as being an expressed antigen on malignant cells, therapeutic targeting of CD34 may result in depletion of healthy stem cell and/or progenitor cell pools.

Described herein are gRNAs that have been developed to specifically direct genetic modification of the gene encoding CD34. Also provided herein is use of such gRNAs to produce genetically modified cells, such as hematopoietic cells, immune cells, lymphocytes, and populations of such cells, that are deficient in CD34 or have reduced expression of CD34 such that the modified cells are not recognized by CD34-specific immunotherapies. Also provided herein are methods involving administering such cells, or compositions thereof, to subjects to address the problem of on-target, off-disease cytotoxicity of certain immunotherapeutic agents. In some examples, as described herein, the genetically modified cells are hematopoietic cells (e.g., HSCs) that are deficient in CD34 or have reduced expression of CD34 that are capable, for example, of developing into progenitor cells or lineage-committed cells. Alternatively or in addition, in some examples, as described herein, the genetically modified cells are immune cells, such as CD34-specific CAR T cells that are deficient in in CD34 or have reduced expression of CD34, and therefore, are resistant to fratricide killing by other CD34-specific CAR T cells.

### Definitions

Antibody: As used herein, the term “antibody” refers to a polypeptide that includes canonical immunoglobulin sequence elements sufficient to confer specific binding to a particular target antigen. As is known in the art, intact antibodies as produced in nature are typically approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)-an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains- an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another, so that the dimers are connected to one another and a tetramer is formed. Naturally-produced antibodies are also typically glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present invention (e.g., as a component of a CAR) include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal. In some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody”, as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity). An antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab2, Fab3, F(ab′)2, Fd, Fv, Feb, scFv, SMIP, single domain antibody, single-chain antibody, diabody, triabody, tetrabody, minibody, maxibody, tandab, DVD, BiTe, TandAb, or the like, or any combination thereof. The subunit structures and three-dimensional configurations of different classes of antibodies are known in the art. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.).

Antigen-binding fragment: An “antigen-binding fragment” refers to a portion of an antibody that binds the antigen to which the antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F (ab′)2; diabodies; single domain antibodies; linear antibodies; single-chain antibody molecules (e.g. scFv or VHH or VH or VL domains only); and multispecific antibodies formed from antibody fragments. In some embodiments, the antigen-binding fragments of the antibodies described herein are scFvs. In some embodiments, the antigen-binding fragments of the antibodies described herein are VHH domains only. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

Antibody heavy chain: As used herein, the term “antibody heavy chain” refers to the larger of the two types of polypeptide chains present in all antibody molecules in their naturally occurring conformations.

Antibody light chain: As used herein, the term “antibody light chain” refers to the smaller of the two types of polypeptide chains present in all antibody molecules in their naturally occurring conformations.

Synthetic antibody: As used herein, the term “synthetic antibody” refers to an antibody that is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Autologous: As used herein, the term “autologous” refers to any material derived from an individual to which it is later to be re-introduced into the same individual.

Allogeneic: As used herein, the term “allogeneic” refers to any material (e.g., a population of cells) derived from a different animal of the same species.

Hyperproliferative disease: As used herein, the term “hyperproliferative disease” refers to a disease characterized by the rapid and uncontrolled growth of aberrant cells. A hyperproliferative disease may be a benign or a malign disease. Malign diseases are typically characterized by the presence of malign cells, e.g., cancer cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the hyperproliferative is a hematopoietic malignancy, such as a myeloid malignancy or a lymphoid malignancy. In some embodiments, the hematopoietic malignancy is acute myeloid leukemia. In some embodiments, the hematopoietic malignancy is myelodysplastic syndrome.

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Co-stimulatory ligand: As used herein, the term “co-stimulatory ligand” refers to a molecule on an antigen presenting cell (e.g., an APC, dendritic cell, B cell, and the like) that specifically binds a cognate co-stimulatory molecule on an immune cell (e.g., a T lymphocyte), thereby providing a signal which mediates an immune cell response, including, but not limited to, proliferation, activation, differentiation, and the like. A co-stimulatory ligand can include, but is not limited to, CD7, B7-1 (CD80), B7-2 (CD86), CD28, PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on an immune cell (e.g., a T lymphocyte), such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

Cytotoxic: As used herein, the term “cytotoxic” or “cytotoxicity” refers to killing or damaging cells. In one embodiment, cytotoxicity of the metabolically enhanced cells is improved, e.g. increased cytolytic activity of immune cells (e.g., T lymphocytes).

Effective amount: As used herein, an “effective amount” as described herein refers to a dose that is adequate to prevent or treat a neoplastic disease, e.g., a cancer, in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the genetically engineered cells of the disclosure (e.g., CAR cells) in each or various rounds of administration, for example in temporal proximity with edited hematopoietic stem cells, as described herein.

For purposes of the invention, the amount or dose of a genetically engineered cell comprising a heterologous nucleic acid comprising a CAR construct described herein that is administered should be sufficient to effect a therapeutic or prophylactic response in the subject or animal over a reasonable time frame. For example, the dose should be sufficient to bind to antigen, or detect, treat, or prevent cancer in a period of from about 2 hours or longer, e.g., about 12 to about 24 or more hours, from the time of administration. In certain embodiments, the time period could be even longer. The dose will be determined by the efficacy of the particular genetically engineered cells of the disclosure (e.g., CAR cells) and the condition of the animal (e.g., human), as well as the body weight of the animal (e.g., human) to be treated.

Effector function: As used herein, “effector function” or “effector activity” refers to a specific activity carried out by an immune cell in response to stimulation of the immune cell. For example, an effector function of a T lymphocyte includes, recognizing an antigen and killing a cell that expresses the antigen.

Endogenous: As used herein “endogenous” refers to any material from or produced inside a particular organism, cell, tissue or system.

Exogenous: As used herein, the term “exogenous” refers to any material introduced from or produced outside a particular organism, cell, tissue or system.

Expand: As used herein, the term “expand” refers to increasing in number, as in an increase in the number of cells, for example, immune cells, e.g., T lymphocytes, B lymphocytes, NK cells, and/or hematopoietic cells. In one embodiment, immune cells, e.g., T lymphocytes, B lymphocytes, NK cells, and/or hematopoietic cells that are expanded ex vivo increase in number relative to the number originally present in a culture. In another embodiment, immune cells, e.g., T lymphocytes, B lymphocytes, NK cells, and/or hematopoietic cells that are expanded ex vivo increase in number relative to other cell types in a culture. In some embodiments, expansion may occur in vivo. The term “ex vivo,” as used herein, refers to cells that have been removed from a living organism, (e.g., a human) and propagated outside the organism (e.g., in a culture dish, test tube, or bioreactor).

Functional Portion: As used herein, the term “functional portion” when used in reference to a CAR refers to any part or fragment of the CAR constructs of the invention, which part or fragment retains the biological activity of the CAR construct of which it is a part (the parent CAR construct). Functional portions encompass, for example, those parts of a CAR construct that retain the ability to recognize target cells, or detect, treat, or prevent cancer, to a similar extent, the same extent, or to a higher extent, as the parent CAR construct. In reference to the parent CAR construct, the functional portion can comprise, for instance, about 10%, about 25%, about 30%, about 50%, about 68%, about 80%, about 90%, about 95%, or more, of the parent CAR.

The functional portion can comprise additional amino acids at the amino or carboxy terminus of the portion, or at both termini, which additional amino acids are not found in the amino acid sequence of the parent CAR construct. Desirably, the additional amino acids do not interfere with the biological function of the functional portion, e.g., recognize target cells, detect cancer, treat or prevent cancer, etc. More desirably, the additional amino acids enhance the biological activity as compared to the biological activity of the parent CAR construct.

Functional Variant: As used herein, the term “functional variant,” as used herein, refers to a CAR construct, polypeptide, or protein having substantial or significant sequence identity or similarity to a parent CAR construct, which functional variant retains the biological activity of the CAR of which it is a variant. Functional variants encompass, for example, those variants of the CAR construct described herein (the parent CAR construct) that retain the ability to recognize target cells to a similar extent, the same extent, or to a higher extent, as the parent CAR construct. In reference to the parent CAR construct, the functional variant can, for instance, be at least about 30%, about 50%, about 75%, about 80%, about 90%, about 91%, about 92%, about 93%, about 94%, about 95%, about 96%, about 97%, about 98%, about 99% or more identical in amino acid sequence to the parent CAR construct. A functional variant can, for example, comprise the amino acid sequence of the parent CAR with at least one conservative amino acid substitution. Alternatively or additionally, the functional variants can comprise the amino acid sequence of the parent CAR construct with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR construct.

gRNA: The terms “gRNA” and “guide RNA” are used interchangeably throughout and refer to a nucleic acid that promotes the specific targeting or homing of a gRNA/Cas9 molecule complex to a target nucleic acid. A gRNA can be unimolecular (having a single RNA molecule), sometimes referred to herein as sgRNAs, or modular (comprising more than one, and typically two, separate RNA molecules). A gRNA may bind to a target domain in the genome of a host cell. The gRNA (e.g., the targeting domain thereof) may be partially or completely complementary to the target domain. The gRNA may also comprise a “scaffold sequence,” (e.g., a tracrRNA sequence), that recruits a Cas9 molecule to a target domain bound to a gRNA sequence (e.g., by the targeting domain of the gRNA sequence). The scaffold sequence may comprise at least one stem loop structure and recruits an endonuclease. Exemplary scaffold sequences can be found, for example, in Jinek, et al. I (2012) 337(6096):816-821, Ran, et al. Nature Protocols (2013) 8:2281-2308, PCT Publication No. WO2014/093694, and PCT Publication No. WO2013/176772.

Heterologous: As used herein, the term “heterologous” refers to a phenomenon occurring in a living system, e.g., a cell, that does not naturally occur in that system. For example, expression of a protein in a cell, where the protein does not naturally occur in that cell (e.g., the cell does not naturally encode that protein), would be heterologous expression of the protein. In some embodiments, the heterologous nucleic acid encodes a chimeric antigen receptor construct.

Immune cell: As used herein, the term “immune cell,” used interchangeably herein with the term “immune effector cell,” refers to a cell that is involved in an immune response, e.g., promotion of an immune response. Examples of immune cells include, but are not limited to, T-lymphocytes, natural killer (NK) cells, macrophages, monocytes, dendritic cells, neutrophils, eosinophils, mast cells, platelets, large granular lymphocytes, Langerhans' cells, or B-lymphocytes. A source of immune cells (e.g., T lymphocytes, B lymphocytes, NK cells) can be obtained from a subject.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Single chain antibodies: As used herein, the term “single chain antibodies” refers to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

Specifically binds: As used herein, the term “specifically binds,” with respect to an antigen binding domain, such as an antibody agent or a portion of a chimeric antigen receptor, refers to an antigen binding domain or antibody agent which recognizes a specific antigen, but does not substantially recognize or bind other molecules in a sample. For example, an antigen binding domain or antibody agent that specifically binds to an antigen from one species may also bind to that antigen from one or more species. But, such cross-species reactivity does not itself alter the classification of an antigen binding domain or antibody agent as specific. In another example, an antigen binding domain or antibody agent that specifically binds to an antigen may also bind to different allelic forms of the antigen. However, such cross reactivity does not itself alter the classification of an antigen-binding domain or antibody agent as specific. In some instances, the terms “specific binding” or “specifically binding,” can be used in reference to the interaction of an antigen binding domain or antibody agent, a protein, or a peptide with a second chemical species, to mean that the interaction is dependent upon the presence of a particular structure (e.g., an antigenic determinant or epitope) on the chemical species; for example, an antigen binding domain or antibody agent recognizes and binds to a specific protein structure rather than to proteins generally. If an antigen binding domain or antibody agent is specific for epitope “A”, the presence of a molecule containing epitope A (or free, unlabeled A), in a reaction containing labeled “A” and the antigen binding domain or antibody agent, will reduce the amount of labeled A bound to the antibody.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments, a human subject is an adult, adolescent, or pediatric subject. In some embodiments, a subject is suffering from a disease, disorder or condition, e.g., a disease, disorder, or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by, for example, a CAR, as described herein.

Therapeutic: As used herein, the term “therapeutic” refers to a treatment and/or prophylaxis. A therapeutic effect is obtained by suppression, remission, or eradication of a disease state.

Transfected: As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Transgene: As used herein, the term “transgene” refers to an exogenous nucleic acid sequence comprised in a cell, e.g., in the genome of the cell, in which the nucleic acid sequence does not naturally occur. In some embodiments, a transgene may comprise or consist of a nucleic acid sequence encoding a gene product, e.g., a CAR. In some embodiments, a transgene may comprise or consist of an expression construct, e.g., a nucleic acid sequence encoding a gene product under the control of a regulatory element, e.g., a promoter.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition. In some embodiments, treating may comprise administering to a subject an immune cell comprising a genetically engineered cell expressing a CAR (e.g., a T lymphocyte, B-lymphocyte, NK cell) or administering to a subject a hematopoietic stem cell transplant comprising genetically engineered stem cells.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

### Genetically Engineered Cells and Related Compositions and Methods

Some aspects of this disclosure provide genetically engineered cells comprising a modification in their genome that results in a loss of expression of CD34, or expression of a variant form of CD34 that is not recognized by an immunotherapeutic agent targeting CD34. In some embodiments, the modification in the genome of the cell is a mutation in a genomic sequence encoding CD34.

The term “mutation,” as used herein, refers to a change (e.g., an insertion, deletion, inversion, or substitution) in a nucleic acid sequence as compared to a reference sequence, e.g., the corresponding sequence of a cell not having such a mutation, or the corresponding wild-type nucleic acid sequence. In some embodiments provided herein, a mutation in a gene encoding CD34 results in a loss of expression of CD34 in a cell harboring the mutation. In some embodiments, a mutation in a gene encoding CD34 results in the expression of a variant form of CD34 that is not bound by an immunotherapeutic agent targeting CD34, or bound at a significantly lower level than the non-mutated CD34 form encoded by the gene. In some embodiment, a cell harboring a genomic mutation in the CD34 gene as provided herein is not bound by, or is bound at a significantly lower level by an immunotherapeutic agent that targets CD34, e.g., an anti-CD34 antibody or chimeric antigen receptor (CAR).

Some aspects of this disclosure provide compositions and methods for generating the genetically engineered cells described herein, e.g., genetically engineered cells comprising a modification in their genome that results in a loss of expression of CD34, or expression of a variant form of CD34 that is not recognized by an immunotherapeutic agent targeting CD34. Such compositions and methods provided herein include, without limitation, suitable strategies and approaches for genetically engineering cells, e.g., by using RNA-guided nucleases, such as CRISPR/Cas nucleases, and suitable RNAs able to bind such RNA-guided nucleases and target them to a suitable target site within the genome of a cell to effect a genomic modification resulting in a loss of expression of CD34, or expression of a variant form of CD34 that is not recognized by an immunotherapeutic agent targeting CD34.

In some embodiments, a genetically engineered cell (e.g., a genetically engineered hematopoietic cell, such as, for example, a genetically engineered hematopoietic stem or progenitor cell or a genetically engineered immune effector cell) described herein is generated via genome editing technology, which includes any technology capable of introducing targeted changes, also referred to as “edits,” into the genome of a cell.

One exemplary suitable genome editing technology is “gene editing,” comprising the use of a RNA-guided nuclease, e.g., a CRISPR/Cas nuclease, to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair (HDR) that typically result in an altered nucleic acid sequence (e.g., via nucleotide or nucleotide sequence insertion, deletion, inversion, or substitution) at or immediately proximal to the site of the nuclease cut. See, Yeh et al. Nat. Cell. Biol. (2019) 21:1468-1478; e.g., Hsu et al. Cell (2014) 157:1262-1278; Jasin et al. DNA Repair (2016) 44:6-16; Sfeir et al. Trends Biochem. Sci. (2015) 40:701-714.

Another exemplary suitable genome editing technology is “base editing,” which includes the use of a base editor, e.g., a nuclease-impaired or partially nuclease-impaired RNA-guided CRISPR/Cas protein fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533:420-424; Rees et al. Nat. Rev. Genet. (2018) 19(12): 770-788; Anzalone et al. Nat. Biotechnol. (2020) 38:824-844;

Yet another exemplary suitable genome editing technology includes “prime editing,” which includes the introduction of new genetic information, e.g., an altered nucleotide sequence, into a specifically targeted genomic site using a catalytically impaired or partially catalytically impaired RNA-guided nuclease, e.g., a CRISPR/Cas nuclease, fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157.

The use of genome editing technology typically features the use of a suitable RNA-guided nuclease, which, in some embodiments, e.g., for base editing or prime editing, may be catalytically impaired, or partially catalytically impaired. Examples of suitable RNA-guided nucleases include CRISPR/Cas nucleases. For example, in some embodiments, a suitable RNA-guided nuclease for use in the methods of genetically engineering cells provided herein is a Cas9 nuclease, e.g., an spCas9 or an saCas9 nuclease. For another example, in some embodiments, a suitable RNA-guided nuclease for use in the methods of genetically engineering cells provided herein is a Cas12 nuclease, e.g., a Cas12a nuclease. Exemplary suitable Cas12 nucleases include, without limitation, AsCas12a, FnCas12a, other Cas 12a orthologs, and Cas 12a derivatives, such as the MAD7 system (MAD7™, Inscripta, Inc.), or the Alt-R Cas12a (Cpf1) Ultra nuclease (Alt-R® Cas12a Ultra; Integrated DNA Technologies, Inc.). See, e.g., Gill et al. LIPSCOMB 2017. In United States: Inscripta Inc.; Price et al. Biotechnol. Bioeng. (2020) 117(60): 1805-1816;

In some embodiments, a genetically engineered cell (e.g., a genetically engineered hematopoietic cell, such as, for example, a genetically engineered hematopoietic stem or progenitor cell or a genetically engineered immune effector cell) described herein is generated by targeting an RNA-guided nuclease, e.g., a CRISPR/Cas nuclease, such as, for example, a Cas9 nuclease or a Cas12a nuclease, to a suitable target site in the genome of the cell, under conditions suitable for the RNA-guided nuclease to bind the target site and cut the genomic DNA of the cell. A suitable RNA-guided nuclease can be targeted to a specific target site within the genome by a suitable guide RNA (gRNA). Suitable gRNAs for targeting CRISPR/Cas nucleases according to aspects of this disclosure are provided herein and exemplary suitable gRNAs are described in more detail elsewhere herein.

In some embodiments, a CD34 gRNA described herein is complexed with a CRISPR/Cas nuclease, e.g., a Cas9 nuclease. Various Cas9 nucleases are suitable for use with the gRNAs provided herein to effect genome editing according to aspects of this disclosure, e.g., to create a genomic modification in the CD34 gene. Typically, the Cas nuclease and the gRNA are provided in a form and under conditions suitable for the formation of a Cas/gRNA complex, that targets a target site on the genome of the cell, e.g., a target site within the CD34 gene. In some embodiments, a Cas nuclease is used that exhibits a desired PAM specificity to target the Cas/gRNA complex to a desired target domain in the CD34 gene. Suitable target domains and corresponding gRNA targeting domain sequences are provided herein.

In some embodiments, a Cas/gRNA complex is formed, e.g., in vitro, and a target cell is contacted with the Cas/gRNA complex, e.g., via electroporation of the Cas/gRNA complex into the cell. In some embodiments, the cell is contacted with Cas protein and gRNA separately, and the Cas/gRNA complex is formed within the cell. In some embodiments, the cell is contacted with a nucleic acid, e.g., a DNA or RNA, encoding the Cas protein, and/or with a nucleic acid encoding the gRNA, or both.

In some embodiments, genetically engineered cells as provided herein are generated using a suitable genome editing technology, wherein the genome editing technology is characterized by the use of a Cas9 nuclease. In some embodiments, the Cas9 molecule is of, or derived from, Streptococcus pyogenes (SpCas9), Staphylococcus aureus (SaCas9), or Streptococcus thermophilus (stCas9). Additional suitable Cas9 molecules include those of, or derived from, Neisseria meningitidis (NmCas9), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In some embodiments, catalytically impaired, or partially impaired, variants of such Cas9 nucleases may be used. Additional suitable Cas9 nucleases, and nuclease variants, will be apparent to those of skill in the art based on the present disclosure. The disclosure is not limited in this respect.

In some embodiments, the Cas nuclease is a naturally occurring Cas molecule. In some embodiments, the Cas nuclease is an engineered, altered, or modified Cas molecule that differs, e.g., by at least one amino acid residue, from a reference sequence, e.g., the most similar naturally occurring Cas9 molecule or a sequence of Table 50 of PCT Publication No. WO2015/157070, which is herein incorporated by reference in its entirety.

In some embodiments, a Cas nuclease is used that belongs to class 2 type V of Cas nucleases. Class 2 type V Cas nucleases can be further categorized as type V-A, type V-B, type V-C, and type V-U. See, e.g., Stella et al. Nature Structural & Molecular Biology (2017). In some embodiments, the Cas nuclease is a type V-B Cas endonuclease, such as a C2c1. See, e.g., Shmakov et al. Mol Cell (2015) 60:385-397. In some embodiments, the Cas nuclease used in the methods of genome editing provided herein is a type V-A Cas endonuclease, such as a Cpf1 (Cas12a) nuclease. See, e.g., Strohkendl et al. Mol. Cell (2018) 71:1-9. In some embodiments, a Cas nuclease used in the methods of genome editing provided herein is a Cpf1 nuclease derived from Provetella spp. or Francisella spp., Acidaminococcus sp. (AsCpf1), Lachnospiraceae bacterium (LpCpf1), or Eubacterium rectale. In some embodiments, the Cas nuclease is MAD7.

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure.

Some features of some exemplary, non-limiting suitable Cas nucleases are described in more detail herein, without wishing to be bound to any particular theory.

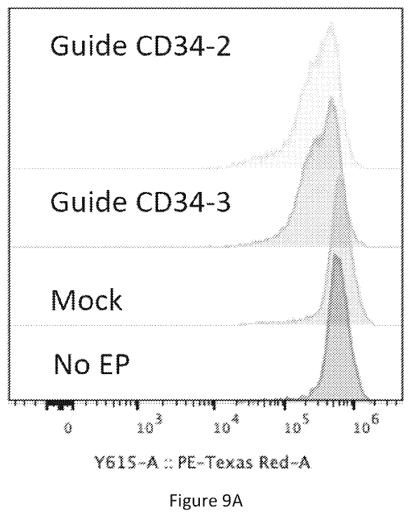

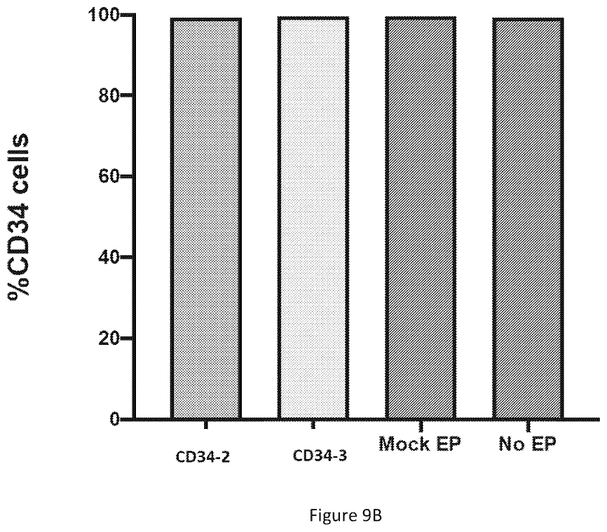

A naturally occurring Cas9 nuclease typically comprises two lobes: a recognition (REC) lobe and a nuclease (NUC) lobe; each of which further comprises domains described, e.g., in PCT Publication No. WO2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat: anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule and comprises amino acids 1099-1368 of the sequence of S. pyogenes Cas9.

Crystal structures have been determined for naturally occurring bacterial Cas9 nucleases (see, e.g., Jinek et al., Science, 343(6176): 1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/nature13579).

In some embodiments, a Cas9 molecule described herein exhibits nuclease activity that results in the introduction of a double strand DNA break in or directly proximal to a target site. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas nuclease or a Cas/gRNA complex described herein is administered together with a template for homology directed repair (HDR). In some embodiments, a Cas nuclease or a Cas/gRNA complex described herein is administered without a HDR template.

In some embodiments, a Cas9 nuclease is used that is modified to enhance specificity of the enzyme (e.g., reduce off-target effects, maintain robust on-target cleavage). In some embodiments, the Cas9 molecule is an enhanced specificity Cas9 variant (e.g., cSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529:490-495.

Various Cas nucleases are known in the art and may be obtained from various sources and/or engineered/modified to modulate one or more activities or specificities of the enzymes. PAM sequence preferences and specificities of suitable Cas nucleases, e.g., suitable Cas9 nucleases, such as, for example, spCas9 and saCas9 are known in the art. In some embodiments, the Cas nuclease has been engineered/modified to recognize one or more PAM sequence. In some embodiments, the Cas nuclease has been engineered/modified to recognize one or more PAM sequence that is different than the PAM sequence the Cas nuclease recognizes without engineering/modification. In some embodiments, the Cas nuclease has been engineered/modified to reduce off-target activity of the enzyme.

In some embodiments, a Cas nuclease is used that is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168:20-36. In some embodiments, a Cas nuclease is used that is modified to alter the PAM recognition or preference of the endonuclease. For example, SpCas9 recognizes the PAM sequence NGG, whereas some variants of SpCas9 comprising one or more modifications (e.g., VQR SpCas9, EQR SpCas9, VRER SpCas9) may recognize variant PAM sequences, e.g., NGA, NGAG, and/or NGCG. For another example, SaCas9 recognizes the PAM sequence NNGRRT, whereas some variants of SaCas9 comprising one or more modifications (e.g., KKH SaCas9) may recognize the PAM sequence NNNRRT. In another example, FnCas9 recognizes the PAM sequence NNG, whereas a variant of the FnCas9 comprises one or more modifications (e.g., RHA FnCas9) may recognize the PAM sequence YG. In another example, the Cas12a nuclease comprising substitution mutations S542R and K607R recognizes the PAM sequence TYCV. In another example, a Cpf1 endonuclease comprising substitution mutations S542R, K607R, and N552R recognizes the PAM sequence TATV. See, e.g., Gao et al. Nat. Biotechnol. (2017) 35(8): 789-792.

In some embodiments, a base editor is used to create a genomic modification resulting in a loss of expression of CD34, or in expression of a CD34 variant not targeted by an immunotherapy. Base editors typically comprise a catalytically inactive or partially inactive Cas nuclease fused to a functional domain, e.g., a deaminase domain. See, e.g., Eid et al. Biochem. J. (2018) 475(11): 1955-1964; Rees et al. Nature Reviews Genetics (2018) 19:770-788. In some embodiments, a catalytically inactive Cas nuclease is referred to as “dead Cas” or “dCas.” In some embodiments, the endonuclease comprises a dCas fused to an adenine base editor (ABE), for example an ABE evolved from the RNA adenine deaminase TadA. In some embodiments, the endonuclease comprises a dCas fused to cytidine deaminase enzyme (e.g., APOBEC deaminase, pmCDA1, activation-induced cytidine deaminase (AID)). In some embodiments, the catalytically inactive Cas molecule has reduced activity and is, e.g., a nickase.

Examples of suitable base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa (KKH)-BE3, Target-AID, Target-AID-NG, xBE3, cA3A-BE3, BE-PLUS, TAM, CRISPR-X, ABE7.9, ABE7.10, ABE7.10*, xABE, ABESa, VQR-ABE, VRER-ABE, Sa (KKH)-ABE, and CRISPR-SKIP. Additional examples of base editors can be found, for example, in US Publication No. 2018/0312825A1, US Publication No. 2018/0312828A1, and PCT Publication No. WO 2018/165629A1, which are incorporated by reference herein in their entireties.

Some aspects of this disclosure provide guide RNAs that are suitable to target an RNA-guided nuclease, e.g. as provided herein, to a suitable target site in the genome of a cell in order to effect a modification in the genome of the cell that results in a loss of expression of CD34, or expression of a variant form of CD34 that is not recognized by an immunotherapeutic agent targeting CD34.

The terms “guide RNA” and “gRNA” are used interchangeably herein and refer to a nucleic acid, typically an RNA, that is bound by an RNA-guided nuclease and promotes the specific targeting or homing of the RNA-guided nuclease to a target nucleic acid, e.g., a target site within the genome of a cell. A gRNA typically comprises at least two domains: a “binding domain,” also sometimes referred to as “gRNA scaffold” or “gRNA backbone” that mediates binding to an RNA-guided nuclease (also referred to as the “binding domain”), and a “targeting domain” that mediates the targeting of the gRNA-bound RNA-guided nuclease to a target site. Some gRNAs comprise additional domains, e.g., complementarity domains, or stem-loop domains. The structures and sequences of naturally occurring gRNA binding domains and engineered variants thereof are well known to those of skill in the art. Some suitable gRNAs are unimolecular, comprising a single nucleic acid sequence, while other suitable gRNAs comprise two sequences (e.g., a crRNA and tracrRNA sequence).

Some exemplary suitable Cas9 gRNA scaffold sequences are provided herein, and additional suitable gRNA scaffold sequences will be apparent to the skilled artisan based on the present disclosure. Such additional suitable scaffold sequences include, without limitation, those recited in Jinek, et al. Science (2012) 337(6096):816-821, Ran, et al. Nature Protocols (2013) 8:2281-2308, PCT Publication No. WO2014/093694, and PCT Publication No. WO2013/176772.

For example, the binding domains of naturally occurring spCas9 gRNA typically comprise two RNA molecules, the crRNA (partially) and the tracrRNA. Variants of spCas9 gRNAs that comprise only a single RNA molecule including both crRNA and tracrRNA sequences, covalently bound to each other, e.g., via a tetraloop or via click-chemistry type covalent linkage, have been engineered and are commonly referred to as “single guide RNA” or “sgRNA.” Suitable gRNAs for use with other Cas nucleases, for example, with Cas12a nucleases, typically comprise only a single RNA molecule, as the naturally occurring Cas12a guide RNA comprises a single RNA molecule. A suitable gRNA may thus be unimolecular (having a single RNA molecule), sometimes referred to herein as sgRNAs, or modular (comprising more than one, and typically two, separate RNA molecules).

A gRNA suitable for targeting a target site in the CD34 gene may comprise a number of domains. In some embodiments, e.g., in some embodiments where a Cas9 nuclease is used, a unimolecular sgRNA, may comprise, from 5′ to 3′:
a targeting domain corresponding to a target site sequence in the CD34 gene;
a first complementarity domain;
a linking domain;
a second complementarity domain (which is complementary to the first complementarity domain);
a proximal domain; and
optionally, a tail domain.

Each of these domains is now described in more detail.

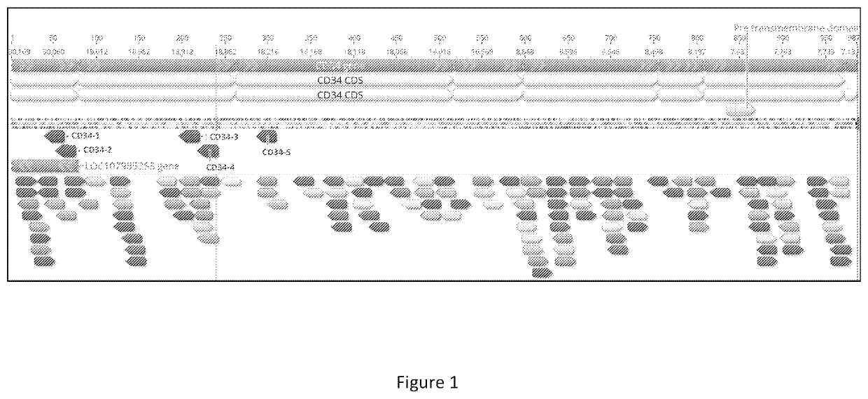

A gRNA as provided herein typically comprises a targeting domain that binds to a target site in the genome of a cell. The target site is typically a double-stranded DNA sequence comprising the PAM sequence and, on the same strand as, and directly adjacent to, the PAM sequence, the target domain. The targeting domain of the gRNA typically comprises an RNA sequence that corresponds to the target domain sequence in that it resembles the sequence of the target domain, sometimes with one or more mismatches, but typically comprises an RNA instead of a DNA sequence. The targeting domain of the gRNA thus base-pairs (in full or partial complementarity) with the sequence of the double-stranded target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., FIG. 1 of Vanegas et al., Fungal Biol Biotechnol. 2019; 6: 6, which is incorporated by reference herein. For additional illustration and description of the mechanism of gRNA targeting an RNA-guided nuclease to a target site, see Fu Y et al, Nat Biotechnol 2014 (doi: 10.1038/nbt.2808) and Sternberg S H et al., Nature 2014 (doi: 10.1038/nature13011), both incorporated herein by reference.

The targeting domain may comprise a nucleotide sequence that corresponds to the sequence of the target domain, i.e., the DNA sequence directly adjacent to the PAM sequence (e.g., 5′ of the PAM sequence for Cas9 nucleases, or 3′ of the PAM sequence for Cas12a nucleases). The targeting domain sequence typically comprises between 17 and 30 nucleotides and corresponds fully with the target domain sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below. Top to bottom, left to right the sequences correspond to SEQ ID NOs 24-26:

[ target domain (DNA) ][ PAM ]
5'-N-N-N-N-N-N-N-N-N-N-N-N-N-N-N-N-N-N-N-N-N-N-N-G-G-3' (DNA)
3'-N-N-N-N-N-N-N-N-N-N-N-N-N-N-N-N-N-N-N-N-N-N-N-C-C-5' (DNA)
| | | | | | | | | | | | | | | | | | | | |
5'-N-N-N-N-N-N-N-N-N-N-N-N-N-N-N-N-N-N-N-N-N-N-[gRNA scaffold]-3' (RNA)
[ target domain (RNA) ] [binding domain]

An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below. Top to bottom, left to right the sequences correspond to SEQ ID NOs 27, 28, and 26:

```
                    [ PAM ][ target domain (DNA) ]
                    5'-T-T-N-N-N-N-N-N-N-N-N-N-N-N-N-N-N-N-N-N-N-N-N-N-N-3' (DNA)
                    3'-A-A-N-N-N-N-N-N-N-N-N-N-N-N-N-N-N-N-N-N-N-N-N-N-N-5' (DNA)
                                      | | | | | | | | | | | | | | | | | | | | |
5'-[gRNA scaffold]-N-N-N-N-N-N-N-N-N-N-N-N-N-N-N-N-N-N-N-N-N-N-3' (RNA)
    [binding domain][ target domain (RNA) ]
```

In some embodiments, the Cas12a PAM sequence is 5′-T-T-T-V-3′.

While not wishing to be bound by theory, at least in some embodiments, it is believed that the length and complementarity of the targeting domain with the target sequence contributes to specificity of the interaction of the gRNA/Cas9 molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in PCT Publication No. WO2015/157070, which is incorporated by reference in its entirety. In some embodiments, the core domain comprises about 8 to about 13 nucleotides from the 3′ end of the targeting domain (e.g., the most 3′8 to 13 nucleotides of the targeting domain). In some embodiments, the secondary domain is positioned 5′ to the core domain. In some embodiments, the core domain corresponds fully with the target domain sequence, or a part thereof. In other embodiments, the core domain may comprise one or more nucleotides that are mismatched with the corresponding nucleotide of the target domain sequence.

In some embodiments, e.g., in some embodiments where a Cas9 gRNA is provided, the gRNA comprises a first complementarity domain and a second complementarity domain, wherein the first complementarity domain is complementary with the second complementarity domain, and, at least in some embodiments, has sufficient complementarity to the second complementarity domain to form a duplexed region under at least some physiological conditions. In some embodiments, the first complementarity domain is 5 to 30 nucleotides in length. In some embodiments, the first complementarity domain comprises 3 subdomains, which, in the 5′ to 3′ direction are: a 5′ subdomain, a central subdomain, and a 3′ subdomain. In some embodiments, the 5′ subdomain is 4 to 9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in PCT Publication No. WO2015/157070, which is herein incorporated by reference in its entirety, including p. 88-112 therein.

A linking domain may serve to link the first complementarity domain with the second complementarity domain of a unimolecular gRNA. The linking domain can link the first and second complementarity domains covalently or non-covalently. In some embodiments, the linkage is covalent. In some embodiments, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO2018/126176, the entire contents of which are incorporated herein by reference.

In some embodiments, the second complementarity domain is complementary, at least in part, with the first complementarity domain, and in an embodiment, has sufficient complementarity to the second complementarity domain to form a duplexed region under at least some physiological conditions. In some embodiments, the second complementarity domain can include a sequence that lacks complementarity with the first complementarity domain, e.g., a sequence that loops out from the duplexed region. In some embodiments, the second complementarity domain is 5 to 27 nucleotides in length. In some embodiments, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5, e.g., 3 nucleotides in length. In some embodiments, the 3′ subdomain is 4 to 9, e.g., 4, 5, 6, 7, 8 or 9 nucleotides in length. In some embodiments, the 5′ subdomain and the 3′ subdomain of the first complementarity domain, are respectively, complementary, e.g., fully complementary, with the 3′ subdomain and the 5′ subdomain of the second complementarity domain.

In some embodiments, the proximal domain is 5 to 20 nucleotides in length. In some embodiments, the proximal domain can share homology with or be derived from a naturally occurring proximal domain. In an embodiment, it has at least 50% homology with a proximal domain from S. pyogenes, S. aureus, or S. thermophilus.

A broad spectrum of tail domains are suitable for use in gRNAs. In some embodiments, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, the tail domain has at least 50% homology/identity with a tail domain from S. pyogenes, S. aureus or S. thermophilus. In some embodiments, the tail domain includes nucleotides at the 3′ end that are related to the method of in vitro or in vivo transcription.

In some embodiments, a gRNA provided herein comprises:
a first strand comprising, e.g., from 5′ to 3′:
   a targeting domain (which corresponds to a target domain in the CD34 gene); and
   a first complementarity domain; and
a second strand, comprising, e.g., from 5′ to 3′:
   optionally, a 5′ extension domain;
   a second complementarity domain;
   a proximal domain; and
   optionally, a tail domain.

In some embodiments, any of the gRNAs provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable gRNA modifications will be apparent to the skilled artisan based on this disclosure, and such suitable gRNA modifications include, without limitation, those described, e.g., in Rahdar et al. PNAS (2015) 112 (51) E7110-E7117 and Hendel et al., Nat Biotechnol. (2015); 33(9): 985-989, each of which is incorporated herein by reference in its entirety.

For example, a gRNA provided herein may comprise one or more 2′-O modified nucleotide, e.g., a 2′-O-methyl nucleotide. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., a 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, a gRNA provided herein may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, a gRNA provided herein comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

In some embodiments, a gRNA described herein comprises one or more 2′-O-methyl-3′-phosphorothioate nucleotides, e.g., at least 1, 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the gRNA may comprise one or more modified nucleotides, e.g., as described in PCT Publication Nos. WO2017/214460, WO2016/089433, and WO2016/164356, which are incorporated by reference their entirety.

The CD34 targeting gRNAs provided herein can be delivered to a cell in any manner suitable. Various suitable methods for the delivery of CRISPR/Cas systems, e.g., comprising an RNP including a gRNA bound to an RNA-guided nuclease, have been described, and exemplary suitable methods include, without limitation, electroporation of RNP into a cell, electroporation of mRNA encoding a Cas nuclease and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

The present disclosure provides a number of CD34 target sites and corresponding gRNAs that are useful for targeting an RNA-guided nuclease to human CD34. Table 1 below illustrates preferred target domains in the human endogenous CD34 gene that can be bound by gRNAs described herein. The exemplary target sequences of human CD34 shown in Table 1, in some embodiments, are for use with a Cas9 nuclease, e.g., SpCas9.

TABLE 1
Exemplary Cas9 target site sequences of human
CD34 are provided, as are exemplary gRNA
targeting domain sequences useful for
targeting such sites. For each target site,
the first sequence represents the DNA target
domain sequence, the second sequence represents
the reverse complement thereof, and the third
sequence represents an exemplary targeting
domain sequence of a gRNA that can be used
to target the respective target site.

| gRNA Name | Target domain sequence |
|---|---|
| CD34-1 | CAAAGCGCGGTCCAGCCCCG (SEQ ID NO: 1) |
| | CGGGGCTGGACCGCGCTTTG (SEQ ID NO: 6) |
| | CAAAGCGCGGUCCAGCCCCG (SEQ ID NO: 11) |
| CD34-2 | CAAACTCAGCAAGCAAAGCG (SEQ ID NO: 2) |
| | CGCTTTGCTTGCTGAGTTTG (SEQ ID NO: 7) |
| | CAAACUCAGCAAGCAAAGCG (SEQ ID NO: 12) |
| CD34-3 | GTGCAGGCTGGTACTTCCAA (SEQ ID NO: 3) |
| | TTGGAAGTACCAGCCTGCAC (SEQ ID NO: 8) |
| | GUGCAGGCUGGUACUUCCAA (SEQ ID NO: 13) |
| CD34-4 | CATTGCCATGTTGAGACACA (SEQ ID NO: 4) |
| | TGTGTCTCAACATGGCAATG (SEQ ID NO: 9) |
| | CAUUGCCAUGUUGAGACACA (SEQ ID NO: 14) |
| CD34-5 | ATAAACTGAGGITATCACAG (SEQ ID NO: 5) |
| | CTGTGATAACCTCAGTTTAT (SEQ ID NO: 10) |
| | AUAAACUGAGGUUAUCACAG (SEQ ID NO: 15) |

The present disclosure provides exemplary CD34 targeting gRNAs that are useful for targeting an RNA-guided nuclease to human CD34. Table 2 below illustrates preferred targeting domains for use in gRNAs targeting Cas9 nucleases to human endogenous CD34 gene. The exemplary target sequences of human CD34 shown in Table 2, in some embodiments, are for use with a Cas9 nuclease, e.g., SpCas9.

TABLE 2
Exemplary Cas9 targeting domain sequences
of gRNAs targeted to human
CD34 are provided.

| gRNA Name | Targeting domain sequence |
|---|---|
| CD34-1 | CAAAGCGCGGUCCAGCCCCG (SEQ ID NO: 11) |
| CD34-2 | CAAACUCAGCAAGCAAAGCG (SEQ ID NO: 12) |
| CD34-3 | GUGCAGGCUGGUACUUCCAA (SEQ ID NO: 13) |
| CD34-4 | CAUUGCCAUGUUGAGACACA (SEQ ID NO: 14) |
| CD34-5 | AUAAACUGAGGUUAUCACAG (SEQ ID NO: 15) |

A representative amino acid sequence of CD34 is provided by UniProtKB/Swiss-Prot Accession No. P28906-2, shown below.

(SEQ ID NO: 16)
MLVRRGARAG PRMPRGWTAL CLLSLLPSGF MSLDNNGTAT PELPTQGTFS
NVSTNVSYQE TTTPSILGST SLHPVSQHGN EATTNITETT VKFTSISVIT
SVYGNINSSV QSQTSVISTV FTTPANVSTP ETTLKPSLSP GNVSDLSTTS
TSLATSPTKP YTSSSPILSD IKAEIKCSGI REVKLTQGIC LEQNKTSSCA
EFKKDRGEGL ARVLCGEEQA DADAGAQVCS LLLAQSEVRP QCLLLVLANR
TEISSKLQLM KKHQSDLKKL GILDFTEQDV ASHQSYSQKT LIALVTSGAL
LAVLGITGYF LMNRRSWSPT GERLGEDPYY TENGGGQGYS SGPGTSPEAQ
GKASVNRGAQ ENGTGQATSR NGHSARQHVV ADTEL

A representative DNA sequence of CD34 is provided by NCBI Reference Sequence No. NM_001025109.2, shown below.

(SEQ ID NO: 17)
agtgtcttcc actcggtgcg tctctctagg agccgcgcgg gaaggatgct ggtccgcagg
ggcgcgcgcg cagggcccag gatgccgcgg ggctggaccg cgctttgctt gctgagtttg
ctgccttctg ggttcatgag tcttgacaac aacggtactg ctaccccaga gttacctacc
cagggaacat tttcaaatgt ttctacaaat gtatcctacc aagaaactac aacacctagt
acccttggaa gtaccagcct gcaccctgtg tctcaacatg gcaatgaggc cacaacaaac
atcacagaaa cgacagtcaa attcacatct acctctgtga taacctcagt ttatggaaac
acaaactctt ctgtccagtc acagacctct gtaatcagca cagtgttcac caccccagcc
aacgtttcaa ctccagagac aaccttgaag cctagcctgt cacctggaaa tgtttcagac
ctttcaacca ctagcactag ccttgcaaca tctcccacta aaccctatac atcatcttct
cctatcctaa gtgacatcaa ggcagaaatc aaatgttcag gcatcagaga agtgaaattg
actcagggca tctgcctgga gcaaaataag acctccagct gtgcggagtt taagaaggac
aggggagagg gcctggcccg agtgctgtgt ggggaggagc aggctgatgc tgatgctggg
gcccaggtat gctccctgct ccttgcccag tctgaggtga ggcctcagtg tctactgctg
gtcttggcca acagaacaga aatttccagc aaactccaac ttatgaaaaa gcaccaatct
gacctgaaaa agctggggat cctagatttc actgagcaag atgttgcaag ccaccagagc
tattcccaaa agaccctgat tgcactggtc acctcgggag ccctgctggc tgtcttgggc
atcactggct atttcctgat gaatcgccgc agctggagcc ccacaggaga aaggctgggc
gaagaccctt attacacgga aaacggtgga ggccagggct atagctcagg acctgggacc
tcccctgagg ctcagggaaa ggccagtgtg aaccgagggg ctcaggaaaa cgggaccggc
caggccacct ccagaaacgg ccattcagca agacaacacg tggtggctga taccgaattg
tgactcggct aggtggggca aggctgggca gtgtccgaga gagcacccct ctctgcatct
gaccacgtgc tacccccatg ctggaggtga catctcttac gcccaaccct tccccactgc
acacacctca gaggctgttc ttggggccct acaccttgag gaggggcagg taaactcctg
tcctttacac attcggctcc ctggagccag actctggtct tctttgggta aacgtgtgac
gggggaaagc caaggtctgg agaagctccc aggaacaatc gatggccttg cagcactcac
acaggacccc cttcccctac cccctcctct ctgccgcaat acaggaaccc ccaggggaaa
gatgagcttt tctaggctac aattttctcc caggaagctt tgatttttac cgtttcttcc
ctgtattttc tttctctact ttgaggaaac caaagtaacc ttttgcacct gctctcttgt
aatgatatag ccagaaaaac gtgttgcctt gaaccacttc cctcatctct cctccaagac
actgtggact tggtcaccag ctcctccctt gttctctaag ttccactgag ctccatgtgc
cccctctacc atttgcagag tcctgcacag ttttctggct ggagcctaga acaggcctcc
caagttttag gacaaacagc tcagttctag tctctctggg gccacacaga aactcttttt
gggctccttt ttctccctct ggatcaaagt aggcaggacc atgggaccag gtcttggagc
tgagcctctc acctgtactc ttccgaaaaa tcctcttcct ctgaggctgg atcctagcct
tatcctctga tctccatggc ttcctcctcc ctcctgccga ctcctgggtt gagctgttgc
ctcagtcccc caacagatgc ttttctgtct ctgcctccct caccctgagc cccttccttg
ctctgcaccc ccatatggtc atagcccaga tcagctccta acccttatca ccagctgcct
cttctgtggg tgacccaggt ccttgtttgc tgttgatttc tttccagagg ggttgagcag
ggatcctggt ttcaatgacg gttggaaata gaaatttcca gagaagagag tattgggtag
atattttttc tgaatacaaa gtgatgtgtt taaatactgc aattaaagtg atactgaaac
acatctgtta tgtgactctg tcttagctgg gtgtgtctgc atgcaagagt gacaccctcc
attagaccta gctagactgt gcagtgatgt ggtggggagg accagccagg gaagagggag
cacctcagca gacacaggca ccagccagga tgctaaggac ctttagccaa gtctgccaac
tattctcctc catggggaga ggaaacatcc atttccagtg gtagaaaggc agacccgaat
gtaccaggga gcttccaaat ggagggtggt atgttgggtt cttaggagct gtacccttca
tgaacaccct tctgagaaga ggagcatgct gatcactgct gcaaaatatg caaaacaaag
ggaaggggca atgtcctgtg caccctttat tatcaggcca cccccctccc cagcccccca
ggtcagagta gacacagtga aggactatgt ggggactgtt gttctagaga cctggcagcc
aactcaggga gggggctggt ttccaccctc aagattaaga cagcagccta attaaaaaaa
aaatctgtaa gcatgtacct ccccccagct tccaaaacaa cccccacccc acccctacca
ggccatagga agttggggag ggagtgctga ggagctccag gaaacactcc caagtgtgtc
gacagtggca gaggcagttg gggccaaaca aaggttgatt cttccattct tatctccata
aagccagacc tttcccttca gcactcctcc acccccatct ccttcttgct tttctccaac
tcctctaatc ataggttctt ccctaggaca gaggggaggc gaaatgatga ggttcagagt
cttccctcaa aggcgatggc tgccttgagg gttggagcaa aggatgatga gcaaaagacg
atggtaatca gtagggaagt ccagcccact tgcatctagt tgcacatctt gccttgagag
taatccagtg agggtctgtc ccagctagga catcaagtag gaggggtggg ttcagggttc
agattcctag gaaatatggg aggagaggaa aaggcaactt ggatgcacct ccagcttcag
gcctagcaac ctgcaatgca tctcaccctg agtttgctgg aatgtgtatg tatgctttgg
gaggaagggc tgtgtgtgta ttgcggggtg gggtggggca gctggttccc tctgacagct
ggacagcttg ccctgaagaa tttgcctgct ttctggaaaa atccaacttt cccaccgtgg
gcctgagcgt cctggtacag caatggcgcc acctgctggc cttattgagg tcctactgct
cagcctcagc tcaatcgcct ccatgttggg cttctctccc tggctgcccc accctctagt
ccaatttctc ttgtacacaa agctcatata actatagaac gtcactgttg aagagaactt
taaagataca tttaattaaa ctcccttatg gtatagttaa agacaaacta aggctcagag
aagggaggtg gcttgcccaa tcacccagaa ttccaaagtc ctgaatctgt agttttccct
tccatcatat catcctactc ttctgccgag tcctccgtgt tactccagtt ggatgtcatg
aagccagtgt ggcagtgtga agataggttt gggacttcac ttctggagca tttcatcaac
ataagctatc ctaggcctgg ccagccaagc aggtcctgga ggagccccag gacaaagatc
acaggaggcc atgaggttcg gcttcttcgg cgcccacagt gagcccagga aaattagctg
tagggtatta cactgttgac tatggagagc atatctggaa ttatcttcag ccagattttc
atctgaatgg ataaatggga ataccatcta agtccagata aatagatcac ttccatctca
tcccttctag gtagattaat cccacacttc ctcttcacac aaaaccagta ataggtcatc
gattttgtgc aacaggatgc tgcttctctt cctaaagccc ccatcgaaga ggcttccagc
caccattcaa tcattcatca agtcttatga tgtgccagac actgcgcgaa atgtgccaga
acatctgtta tgtgccagac actgttcttg agactgggga tacagcaaac actcatgaag
cttataattc tagcagaaga ggacagtaaa caatgtcatc tcagtaagta tatacatgtg
ttttcaggat tgagagctat gaaaaacata aaatatattg agaataatgg ttggtatttt
acatatggtg gttactttta gaaaaataac agtggagagc acagcttcac ttgaatgaag
tggagaagca ggttgtatgc caagctggga gagattatcc cacacagggg aaaggacaag
tgcaaagccc tatgatgaaa agctgccaag tgcagaaagc ctcagatggc agggggcaag
atggccatga ggttgtgtca gtgagtgggg gtggggagag gcaggaggtc agactacatg
gggccttttt agttgtagat tgggaagcca ctggagggtt ttgagcagag aagtcatatc
atctgcttta tgttttaaaa ggatcatgct ggctgctgag tagagaatag aggttgaggg
ataagaaagt agaaggagac cgtagcaaga agaacgatca tggctgggag caggtgatca
tattggcagt gatgagatca agcagaattc aaaaagtggt ttcaaagtag aggtaacagg
acttgctcag tctatttatt tcttcaaata ataatcatat ttacaatgat agtagctaac
agtttttgag tgcttactgt atgaaaattg agatatggtg ccaatattta aatagcatat
tttacttaac attcacagaa accctgtgaa gtaggttcta ttatctcaga aaaagaaact
gaaactcaga gaataacaag ggactgtgtt acgtgcacag tggcagaggc aaagatgaat
aggatgtgag tttatttgaa ccccaaatgt ttaaatcttg gggataatac aacacacatt
taaacaaaga agcaagaaaa aaaatgcaca acagaaagtg agaaataaca cgaggaaaga
ctaaatgaag tgctttgtat ctagatgtgg gcaggaccct ttccagctga gaagatctga
gactgggtca tgaacaggtg gtttctgagt gggtcctgta aaaatgaata cgattttgat
gatagtaatg agtaaggaca tttgagactg atagaagagt acatacaata tgtagtgatg
gggaaagata aggtactgtc aaaggacaat gtgttttctg gtatgacaga gaagtagaat
gtgttaaggg aagccgagta ccagaaagat ccgggtgtca cagtttgtgt agggtgttta
aagctaaacc acagagttta attttatcca atagaagagg agccacagaa gagtttccat
ttattcatta atttattcat ttattcaaaa aatatttgag tgcttattat aagccaggta
ctatgccagg cacctgggat aagacatagt cccttctgtc aagtctttac attgggtgga
tgtgggaggg acagatgaca gaacaatatg cattgagtgt aagtgctatg gtataggaag
ctctgagtgg gaggggcatg gaagccgtgg aagaccatgg aaggcttccc aggagaagtg
acgtctggac tgatcctttg gtcaagcagg agttaaagag gagaaaagga gagatatggg
tgttcccgag agaggaagaa gccttgtccc aggagcaaag tgagggtgat tgttccagaa
atgtgagtga ttcttttaag gctcaagcaa agcatgtgat tcttctttat accttctatt
tctttgctga gtgtttctgt tcttttgttt caagcatgct gcaattgctc attaaagcat
gtttatgatg gctgtctgtt ttaaaattct tgtcagatgg tttcaacatc tttatcatct
caatgttggc atctgttaat ggttttttct caatcaaatt gagattttcc tggttcttgg
tattaccagt gattttaatt gcatctggaa atttgggatt tatgttgaaa gactggatct
tattgaaaga ttctgtttag cacccctcct ttgataccac actggtgggt ccaggttccc
cattcagctg ttgacacctt cagggcagag aggtgggatg gggtgaaggg ggtacctcat
tattgctggc ccaggttaga agttcaggct tcccagtaga tctctgctga taccaccctg
gtgccatgtc attccttgag tccaaaagtc cctcccaatt ctgccttctt ctctctacat
atcggagtct ccctatgttt gacttatata taatgtccag ggtttttaga gttagttaac
aggaggcata agaaaaagtg tgtccactcc atcttgtctg gaactggaag ttcaagtcga
atataagaga gaggagagga aattacaagc catgagactg gagagttagg caggttctac
accagctatt ctcaaagccc tcttacactc ttaaaaattt agaacttcaa agagcttttg
attttgaaag ttacatctat caattattac tgtttcaaaa attaaaattg agaaaatttt
atttattaat ttgtttaaaa ataacaataa ttattcaatt acatgataat gtaagtaatg
cttttcttaa tgaaaaataa ttatattttc caaaacaaaa acaattagga aaaagagtgt
cattgtttta gactttggta aatctctcta atatctggct gaagagaaga atgctgattc
tttttttttt tttttttttt tgagacggag tctcgctctg tcacccaggc tggagtgtag
tggtgtgatc tcggctcact gcaagctctg cctcccgggt tcacgccatt ctcctgcctc
agcctcccaa gtagctggga ctacaggcac ccgccaccac gcccggctaa tttttttgta
tttttagtag agatggggtt tcaccgtgtt agccaggctg gtctcgatct cctgacctca
tgatccaccc acctcagcct cccaaagcgc tgggattaca ggtgtgagac accgcgccca
gcccccgaat gctgattctt ttatctgctt ctgtattcaa tctgttgtga tatgatgggt
agcctctgaa acactccact gtatacttgt gaaagaatga atgtgaaaaa ggaaaataga
tttgtagtat tattattcaa attgttttga cctcagagac cacttggaaa tgttttaggg
aacccccaga ggaccttgga tcatgctttg agaaccgcgg ctctagatat gttactattt
cagtagcatc taagtacatg tggctgctga gcacttgtaa tgtggctagt gcaaatgaga
gacaggactt ccagctatat gtaatttaat aaactcaaat ttaaaaactg gaacctcata
aaatgttttg ttgttgttgt taaacatgac cttatagttt tggtaggaa

A further representative DNA sequence of CD34 is provided by NCBI Reference Sequence No. NM_001773.3, shown below.

(SEQ ID NO: 18)
agtgtcttcc actcggtgcg tctctctagg agccgcgcgg gaaggatgct ggtccgcagg
ggcgcgcgcg cagggcccag gatgccgcgg ggctggaccg cgctttgctt gctgagtttg
ctgccttctg ggttcatgag tcttgacaac aacggtactg ctaccccaga gttacctacc
cagggaacat tttcaaatgt ttctacaaat gtatcctacc aagaaactac aacacctagt
acccttggaa gtaccagcct gcaccctgtg tctcaacatg gcaatgaggc cacaacaaac
atcacagaaa cgacagtcaa attcacatct acctctgtga taacctcagt ttatggaaac
acaaactctt ctgtccagtc acagacctct gtaatcagca cagtgttcac caccccagcc
aacgtttcaa ctccagagac aaccttgaag cctagcctgt cacctggaaa tgtttcagac
ctttcaacca ctagcactag ccttgcaaca tctcccacta aaccctatac atcatcttct
cctatcctaa gtgacatcaa ggcagaaatc aaatgttcag gcatcagaga agtgaaattg
actcagggca tctgcctgga gcaaaataag acctccagct gtgcggagtt taagaaggac
aggggagagg gcctggcccg agtgtgtgt ggggaggagc aggctgatgc tgatgctggg
gcccaggtat gctccctgct ccttgcccag tctgaggtga ggcctcagtg tctactgctg
gtcttggcca acagaacaga aatttccagc aaactccaac ttatgaaaaa gcaccaatct
gacctgaaaa agctggggat cctagatttc actgagcaag atgttgcaag ccaccagagc
tattcccaaa agaccctgat tgcactggtc acctcgggag ccctgctggc tgtcttgggc
atcactggct atttcctgat gaatcgccgc agctggagcc ccacaggaga aaggctggag
ctggaaccct gaccactctt caggaagaaa ggagtctgca catgcagctg caccctccct
ccgatccttc ctcccacctc cccctccccc ttctcccacc cctgccccca cttcctgttt
gggcccctct cccatccagt gtctcacagc cctgcttacc agataatgct actttattta
tacactgtct agggcgaaga cccttattac acggaaaacg gtggaggcca gggctatagc
tcaggacctg ggacctcccc tgaggctcag ggaaaggcca gtgtgaaccg aggggctcag
gaaaacggga ccggccaggc cacctccaga aacggccatt cagcaagaca acacgtggtg
gctgataccg aattgtgact cggctaggtg gggcaaggct gggcagtgtc cgagagagca
cccctctctg catctgacca cgtgctaccc ccatgctgga ggtgacatct cttacgccca
acccttcccc actgcacaca cctcagaggc tgttcttggg gccctacacc ttgaggaggg
gcaggtaaac tcctgtcctt tacacattcg gctccctgga gccagactct ggtcttcttt
gggtaaacgt gtgacggggg aaagccaagg tctggagaag ctcccaggaa caatcgatgg
ccttgcagca ctcacacagg acccccttcc cctaccccct cctctctgcc gcaatacagg
aacccccagg ggaaagatga gcttttctag gctacaattt tctcccagga agctttgatt
tttaccgttt cttccctgta ttttctttct ctactttgag gaaaccaaag taaccttttg
cacctgctct cttgtaatga tatagccaga aaaacgtgtt gccttgaacc acttccctca
tctctcctcc aagacactgt ggacttggtc accagctcct cccttgttct ctaagttcca
ctgagctcca tgtgccccct ctaccatttg cagagtcctg cacagttttc tggctggagc
ctagaacagg cctcccaagt tttaggacaa acagctcagt tctagtctct ctggggccac
acagaaactc tttttgggct cctttttctc cctctggatc aaagtaggca ggaccatggg
accaggtctt ggagctgagc ctctcacctg tactcttccg aaaaatcctc ttcctctgag
gctggatcct agccttatcc tctgatctcc atggcttcct cctccctcct gccgactcct
gggttgagct gttgcctcag tcccccaaca gatgcttttc tgtctctgcc tccctcaccc
tgagcccctt ccttgctctg cacccccata tggtcatagc ccagatcagc tcctaaccct
tatcaccagc tgcctcttct gtgggtgacc caggtccttg tttgctgttg atttctttcc
agaggggttg agcagggatc ctggtttcaa tgacggttgg aaatagaaat ttccagagaa
gagagtattg ggtagatatt ttttctgaat acaaagtgat gtgtttaaat actgcaatta
aagtgatact gaaacaca

Some aspects of this disclosure provide genetically engineered cells comprising a modification in their genome that results in a loss of expression of CD34, or expression of a variant form of CD34 that is not recognized by an immunotherapeutic agent targeting CD34. In some embodiments, the modification in the genome of the cell is a mutation in a genomic sequence encoding CD34. In some embodiments, the modification is effected via genome editing, e.g., using a Cas nuclease and a gRNA targeting a CD34 target site provided herein or comprising a targeting domain sequence provided herein.

While the compositions, methods, strategies, and treatment modalities provided herein may be applied to any cell or cell type, some exemplary cells and cell types that are particularly suitable for genomic modification in the CD34 gene according to aspects of this invention are described in more detail herein. The skilled artisan will understand, however, that the provision of such examples is for the purpose of illustrating some specific embodiments, and additional suitable cells and cell types will be apparent to the skilled artisan based on the present disclosure, which is not limited in this respect.

Some aspects of this disclosure provide genetically engineered hematopoietic cells comprising a modification in their genome that results in a loss of expression of CD34, or expression of a variant form of CD34 that is not recognized by an immunotherapeutic agent targeting CD34. In some embodiments, the genetically engineered cells comprising a modification in their genome results in reduced cell surface expression of CD34 and/or reduced binding by an immunotherapeutic agent targeting CD34, e.g., as compared to a hematopoietic cell (e.g., HSC) of the same cell type but not comprising a genomic modification. In some embodiments, a hematopoietic cell is a hematopoietic stem cell (HSC). In some embodiments, the hematopoietic cell is a hematopoietic progenitor cell (HPC). In some embodiments, the hematopoietic cell is a hematopoietic stem or progenitor cell. As used herein, an HSC refers to a cell capable of self-renewal and which can generate and/or reconstitute all lineages of the hematopoietic system. In some embodiments, an HSC can be engrafted into a subject, wherein the HSC expands and may generate and/or reconstitute all lineages of the hematopoietic system. In some embodiments, an HSC expresses one or more cell-surface markers, e.g., CD34. In some embodiments, a genetically engineered cell (e.g., genetically engineered HSC) described herein does not express one or more cell-surface markers typically associated with HSC identification or isolation, expresses a reduced amount of the cell-surface markers, or expresses a variant cell-surface marker not recognized by an immunotherapeutic agent targeting the cell-surface marker, but nevertheless is capable of self-renewal and can generate and/or reconstitute all lineages of the hematopoietic system.

In some embodiments, a hematopoietic cell (e.g., an HSC or HPC) comprising a modification in their genome that results in a loss of expression of CD34, or expression of a variant form of CD34 that is not recognized by an immunotherapeutic agent targeting CD34, is created using a nuclease and/or a gRNA targeting human CD34 as described herein. It will be understood that such a cell can be created by contacting the cell with the nuclease and/or the gRNA, or the cell can be the daughter cell of a cell that was contacted with the nuclease and/or gRNA. In some embodiments, a cell described herein (e.g., a genetically engineered HSC or HPC) is capable of populating the HSC or HPC niche and/or of reconstituting the hematopoietic system of a subject. In some embodiments, a cell described herein (e.g., an HSC or HPC) is capable of one or more of (e.g., all of): engrafting in a human subject, producing myeloid lineage cells, and producing and lymphoid lineage cells. In some preferred embodiments, a genetically engineered hematopoietic cell provided herein, or its progeny, can differentiate into all blood cell lineages, preferably without any differentiation bias as compared to a hematopoietic cell of the same cell type, but not comprising a genomic modification that results in a loss of expression of CD34, or expression of a variant form of CD34 that is not recognized by an immunotherapeutic agent targeting CD34.

In some embodiments, a genetically engineered cell provided herein comprises only one genomic modification, e.g., a genomic modification that results in a loss of expression of CD34, or expression of a variant form of CD34 that is not recognized by an immunotherapeutic agent targeting CD34. It will be understood that the gene editing methods provided herein may result in genomic modifications in one or both alleles of a target gene. In some embodiments, genetically engineered cells comprising a genomic modification in both alleles of a given genetic locus are preferred.

In some embodiments, a genetically engineered cell provided herein comprises two or more genomic modifications, e.g., one or more genomic modifications in addition to a genomic modification that results in a loss of expression of CD34, or expression of a variant form of CD34 that is not recognized by an immunotherapeutic agent targeting CD34.

In some embodiments, a genetically engineered cell provided herein comprises a genomic modification that results in a loss of expression of CD34, or expression of a variant form of CD34 that is not recognized by an immunotherapeutic agent targeting CD34, and further comprises an expression construct that encodes a chimeric antigen receptor, e.g., in the form of an expression construct encoding the CAR integrated in the genome of the cell. In some embodiments, the CAR comprises a binding domain, e.g., an antibody fragment, that binds CD34.

Some aspects of this disclosure provide genetically engineered immune effector cells comprising a modification in their genome that results in a loss of expression of CD34, or expression of a variant form of CD34 that is not recognized by an immunotherapeutic agent targeting CD34. In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT) cell. In some embodiments, the immune effector cell is a natural killer (NK) cell. In some embodiments, the immune effector cell does not express an endogenous transgene, e.g., a transgenic protein. In some embodiments, the immune effector cell expresses a chimeric antigen receptor (CAR). In some embodiments, the immune effector cell expresses a CAR targeting CD34. In some embodiments, the immune effector cell does not express a CAR targeting CD34.

In some embodiments, a genetically engineered cell provided herein comprises a genomic modification that results in a loss of expression of CD34, or expression of a variant form of CD34 that is not recognized by an immunotherapeutic agent targeting CD34, and does not comprise an expression construct that encodes an exogenous protein, e.g., does not comprise an expression construct encoding a CAR.

In some embodiments, a genetically engineered cell provided herein expresses substantially no CD34 protein, e.g., expresses no CD34 protein that can be measured by a suitable method, such as an immunostaining method. In some embodiments, a genetically engineered cell provided herein expresses substantially no wild-type CD34 protein, but expresses a mutant CD34 protein variant, e.g., a variant not recognized by an immunotherapeutic agent targeting CD34, e.g., a CAR-T cell therapeutic, or an anti-CD34 antibody, antibody fragment, or antibody-drug conjugate (ADC).

In some embodiments, the genetically engineered cells provided herein are hematopoietic cells, e.g., hematopoietic stem cells. Hematopoietic cells are typically characterized by pluripotency, self-renewal properties, and/or the ability to generates cells of the hematopoietic system. In some embodiments, hematopoietic stem cells (HSCs) are capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. In some embodiments, HSCs are characterized by the expression of the cell surface marker CD34 (e.g., CD34+). In some embodiments, CD34 can be used for the identification and/or isolation of HSCs. In some embodiments, absence of one or more cell surface markers, e.g., CD34, is associated with commitment to a cell lineage. In some embodiments, a genetically engineered cell described herein (e.g., a genetically engineered HSC) does not express CD34, expresses CD34 at a reduced level, or expresses a CD34 variant (e.g., not recognized by an immunotherapeutic agent targeting CD34). In some such embodiments, e.g., in embodiments where the genetically engineered HSC is characterized by a complete loss of expression of CD34, the genetically engineered cell is not identifiable or isolatable as an HSC by expression of CD34. In some embodiments, a genetically engineered HSC does not express CD34, expresses CD34 at a reduced level, or expresses a CD34 variant (e.g., not recognized by an immunotherapeutic agent targeting CD34) but nevertheless is an HSC capable of self-renewal.

In some embodiments, a genetically engineered HSC disclosed herein, e.g., an HSC that does not express CD34, expresses CD34 at a reduced level, or expresses a CD34 variant, is not identifiable or isolable as an HSC by expression of CD34, but can still be identified as an HSC, e.g., by other characteristics of HSCs. For example, in some embodiments, a genetically engineered HSC as disclosed herein, e.g., an HSC that does not express CD34, expresses CD34 at a reduced level, or expresses a CD34 variant, may be identified as an HSC by the status of its CD34 promoter activity or the epigenetic state of a CD34 promoter in its genome, e.g., via a heterologous reporter construct driven by an endogenous CD34 promoter, or via CD34 promoter methylation status (or other suitable epigentic marker). In some embodiments, a genetically engineered HSC as disclosed herein, e.g., an HSC that does not express CD34, expresses CD34 at a reduced level, or expresses a CD34 variant, may be identified as an HSC by its expression profile of other HSC markers, e.g., in that such expression profile matches or closely resembles that of non-edited HSCs. In some embodiments, a genetically engineered HSC as disclosed herein, e.g., an HSC that does not express CD34, expresses CD34 at a reduced level, or expresses a CD34 variant, may be identified as an HSC by its capacity to engraft into a recipient subject, e.g., a human subject in need thereof, and to re-constitute all hematopoietic cell lineages.

In some embodiments, a population of genetically engineered cells described herein comprises a plurality of genetically engineered hematopoietic stem cells. In some embodiments, a population of genetically engineered cells described herein comprises a plurality of genetically engineered hematopoietic progenitor cells. In some embodiments, a population of genetically engineered cells described herein comprises a plurality of genetically engineered hematopoietic stem cells and a plurality of genetically engineered hematopoietic progenitor cells.

In some embodiments, the genetically engineered HSCs are obtained from a subject, such as a human subject. Methods of obtaining HSCs are described, e.g., in PCT Application No. US2016/057339, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs are obtained from a healthy donor. In some embodiments, the HSCs are obtained from the subject to whom the immune cells expressing the chimeric receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, a population of genetically engineered cells is a heterogeneous population of cells, e.g. heterogeneous population of genetically engineered cells containing different CD34 mutations. In some embodiments, at least 40%, at least 50%, at least 60%, at least 70%, at least 75%, at least 80%, at least 85%, at least 90%, or at least 95% of copies of a gene encoding CD34 in the population of genetically engineered cells comprise a mutation effected by a genome editing approach described herein, e.g., by a CRISPR/Cas system using a gRNA provided herein. By way of example, a population of genetically engineered cells can comprise a plurality of different CD34 mutations and each mutation of the plurality may contribute to the percent of copies of CD34 in the population of cells that have a mutation.

In some embodiments, the expression of CD34 on the genetically engineered hematopoietic cell (e.g., HSC) is compared to the expression of CD34 on a naturally occurring hematopoietic cell (e.g., a wild-type counterpart), e.g., a naturally occurring HSC. In some embodiments, the genetic engineering results in a reduction in the expression level of CD34 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of CD34 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD34 as compared to a naturally occurring hematopoietic cell (e.g., a wild-type counterpart).

In some embodiments, the genetic engineering as described herein, e.g., using a gRNA targeting CD34 as described herein, results in a reduction in the expression level of wild-type CD34 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the level of wild-type CD34 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD34 as compared to a naturally occurring hematopoietic cell (e.g., a wild-type counterpart).

In some embodiments, the genetic engineering as described herein, e.g., using a gRNA targeting CD34 as described herein, results in a reduction in the expression level of wild-type lineage-specific cell surface antigen (e.g., CD34) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to a suitable control (e.g., a cell or plurality of cells). In some embodiments, the suitable control comprises the level of the wild-type lineage-specific cell surface antigen measured or expected in a plurality of non-engineered cells from the same subject. In some embodiments, the suitable control comprises the level of the wild-type lineage-specific cell surface antigen measured or expected in a plurality of cells from a healthy subject. In some embodiments, the suitable control comprises the level of the wild-type lineage-specific cell surface antigen measured or expected in a population of cells from a pool of healthy individuals (e.g., 10, 20, 50, or 100 individuals). In some embodiments, the suitable control comprises the level of the wild-type lineage-specific cell surface antigen measured or expected in a subject in need of a treatment described herein, e.g., an anti-CD34 therapy, e.g., wherein the subject has a cancer, wherein cells of the cancer express CD34.

In some embodiments, a method of genetically engineering cells described herein comprises a step of providing a wild-type cell, e.g., a wild-type hematopoietic stem or progenitor cell. In some embodiments, the wile-type cell is an un-edited cell comprising (e.g., expressing) two functional copies of a gene encoding CD34. In some embodiments, the cell comprises a CD34 gene sequence according to SEQ ID NO: 113. In some embodiments, the cell comprises a CD34 gene sequence encoding a CD34 protein that is encoded in SEQ ID NO: 17 or 18, e.g., the CD34 gene sequence may comprise one or more silent mutations relative to SEQ ID NO: 17 or 18. In some embodiments, the cell used in the method is a naturally occurring cell or a non-engineered cell. In some embodiments, the wild-type cell expresses CD34, or gives rise to a more differentiated cell that expresses CD34 at a level comparable to (or within 90%-110%, 80%-120%, 70%-130%, 60-140%, or 50%-150% of) a cell line expressing CD34.

In some embodiments, the wild-type cell binds an antibody that binds CD34 (e.g., an anti-CD34 antibody), or gives rise to a more differentiated cell that binds such an antibody at a level comparable to (or within 90%-110%, 80%-120%, 70%-130%, 60-140%, or 50%-150% of) binding of the antibody to a cell line expressing CD34, e.g., L1236, L428, KM-H2, and L591). Antibody binding may be measured, for example, by flow cytometry or immunohistochemistry.

In some embodiments, the CD34-edited cells provided herein are CD34-edited hematopoietic stem cells (HSCs). In some embodiments, such CD34-edited hematopoietic stem cells are functionally equivalent to non-edited (e.g., naturally-occurring) HSCs, for example, in that they are capable of long-term engraftment into a recipient, for example in a clinical setting, of differentiating into all hematopoietic lineages, and of reconstituting recipient's hematopoietic system, e.g., after a hematopoietic stem cell transplant. In some embodiments, the CD34-edited HSCs provided herein express cell surface markers characteristic of HSCs, with the exception of CD34. Some suitable cell surface markers associated with hematopoietic stem cells are provided herein, and other suitable surface markers associated with hematopoietic stem cells will be apparent to the skilled artisan based on the present disclosure. Suitable HSC markers include, for example, those disclosed in the BD Biosciences Human and Mouse CD Marker Handbook, accessible at www.bd.com/documents/bd-legacy/catalogue/biosciences/DS_Human-Mouse-CD-Maker-Biosciences_CT_DE.pdf, last accessed Dec. 30, 2021. Additional suitable HSC markers include, for example, those disclosed in Tomellini et al., Cell Reports 2019 28(4):1063-1073 (PMID: 31340144 DOI: 10.1016/j.celrep.2019.06.084), the entire contents of each of which are incorporated herein by reference. Some exemplary suitable HSC markers include, without limitation, CD49c, CD71, CD90, CD117, CD135, CD243, CD292, CDw293, CD318, CD325, CD349, CD201, CD228, and CD309. In some embodiments, suitable HSC surface markers include the absence of surface markers characteristic for differentiated cells. For example, in some embodiments, CD34-edited HSCs as provided herein, e.g., CD34-edited HSCs that are functionally indistinguishable from non-edited (e.g., naturally-occurring) HSCs, are negative for CD34 (CD34−) and express one or more surface markers characteristic of or typically expressed by naturally occurring HSCs, for example, naturally occurring HSCs capable of long-term engraftment into a suitable recipient. In some embodiments, such CD34-edited HSCs as provided herein are CD34− and express CD90 (are CD90+), e.g., as determined by immunostaining pr any other suitable method. In some embodiments, such CD34-edited HSCs as provided herein are negative for CD34 (CD34−) and are CD71+. In some embodiments, such CD34-edited HSCs as provided herein are negative for CD34 (CD34−) and are CD117+. In some embodiments, such CD34-edited HSCs as provided herein are negative for CD34 (CD34−) and are CD135+. In some embodiments, such CD34-edited HSCs as provided herein are negative for CD34 (CD34−) and are CD243+. In some embodiments, such CD34-edited HSCs as provided herein are negative for CD34 (CD34−) and are CD292+. In some embodiments, such CD34-edited HSCs as provided herein are negative for CD34 (CD34−) and are CDw293+. In some embodiments, such CD34-edited HSCs as provided herein are negative for CD34 (CD34−), and are CD318+. In some embodiments, such CD34-edited HSCs as provided herein are negative for CD34 (CD34−) and are CD325+. In some embodiments, such CD34-edited HSCs as provided herein are negative for CD34 (CD34−) and are CD349+. In some embodiments, such CD34-edited HSCs as provided herein are negative for CD34 (CD34−) and are CD201+. In some embodiments, such CD34-edited HSCs as provided herein are negative for CD34 (CD34−) and are CD228+. In some embodiments, such CD34-edited HSCs as provided herein are negative for CD34 (CD34−) and are CD309+. In some embodiments, such CD34-edited HSCs as provided herein are negative for CD34 (CD34−) and are CD71+. In some embodiments, such CD34-edited HSCs as provided herein are negative for CD34 (CD34−) and are CD49c+. In some embodiments, such CD34-edited HSCs as provided herein are negative for CD34 (CD34−) and are CD201+. In some embodiments, such CD34-edited HSCs as provided herein are negative for CD34 (CD34−) and are negative for any lineage marker associated with differentiated hematopoietic cells (lin−). In some embodiments, lineage markers include CD2, CD3, CD4, CD8, CD11b, CD14, CD15, CD16, CD19, CD56, CD123, or CD235a, or any combination of two or more of these markers. Accordingly, in some embodiments, such CD34-edited HSCs as provided herein are negative for CD34 (CD34−) and are negative for CD2, CD3, CD4, CD8, CD11b, CD14, CD15, CD16, CD19, CD56, CD123, or CD235a, or any combination of two or more of these markers. In some embodiments, such CD34-edited HSCs as provided herein are negative for CD34 (CD34−) and are CD45RA−. In some embodiments, such CD34-edited HSCs as provided herein satisfy a combination of two or more of the criteria provided above. For example, in some embodiments, such CD34-edited HSCS are CD34−, CD90+, and CD71+. In some embodiments, such CD34-edited HSCs are CD34−, CD90+, CD71+, and lin−. In some embodiments, such CD34-edited HSCs are CD34−, CD90+, CD45RA−. In some embodiments, such CD34-edited HSCs are CD34−, CD90+, CD45RA−, and CD201+. In some embodiments, such CD34-edited HSCs; are CD34−, CD90+, CD45RA−, and CD49c+; In some embodiments, such CD34-edited HSCs CD34−, CD90+, CD45RA−, CD201+, and CD49c+. In some embodiments, such CD34-edited HSCs CD34−, CD90+, CD45RA−, CD201+, CD49c+, and lin−. In some embodiments, the genetically engineered hematopoietic stem cell does not express CD34 on its cell surface and expresses CD90, CD201, and CD49c. In some embodiments, the genetically engineered hematopoietic stem cell does not express CD34 on its cell surface, expresses CD90, CD201, and CD49c, and does not express CD45c. In some embodiments, the genetically engineered hematopoietic stem cell does not express CD34 on its cell surface and expresses one or more of CD90, CD201, and CD49c, and is negative for CD2, CD3, CD4, CD8, CD11b, CD14, CD15, CD16, CD19, CD45RA−, CD56, CD123, and CD235a.

In some embodiments, CD34-edited HSCs provided herein comprise a genetic edit in the gene encoding CD34 that results in the edited gene not encoding a CD34 gene product, e.g., a CD34 protein, that is expressed on the surface of the CD34-edited HSC, or that results in the edited gene encoding a CD34 protein that cannot be bound by a CD34-targeted antibody (e.g., having a modification in the epitope bound by the CD34-targeted antibody). In some embodiments, the CD34 genetic edit comprises an INDEL that results from an NHEJ-mediated repair of a double-stranded cut in the gene encoding CD34, e.g., from an RNA-guided nuclease, which in turn results in an early termination and thus a truncation of a gene product encoded by CD34, or otherwise in the edited CD34 gene not encoding a CD34 gene product, e.g., a CD34 protein, that is expressed on the surface of the CD34-edited HSC, or that results in the edited gene encoding a CD34 protein that cannot be bound by a CD34-targeted antibody (e.g., having a modification in the epitope bound by the CD34-targeted antibody). In some embodiments, the CD34 genetic edit comprises a single nucleotide change, e.g., resulting from a base edit or a prime edit, or from an HDR-mediated repair of a double-stranded cut in the gene encoding CD34, which in turn results in an early termination and thus a truncation of a gene product encoded by CD34, or otherwise in the edited gene not encoding a CD34 gene product, e.g., a CD34 protein, that is expressed on the surface of the CD34-edited HSC, or that results in the edited gene encoding a CD34 protein that cannot be bound by a CD34-targeted antibody (e.g., having a modification in the epitope bound by the CD34-targeted antibody). In some embodiments, the CD34 genetic edit results in a frame shift or in a splice variant. In some embodiments, the CD34 genetic edit results in a deletion, in full or in part, or in a modification, of an epitope bound be a CD34-antibody. In some embodiments, the CD34-edited HSCs are not recognized by a CD34-targeted antibody, CD34-binding antibody fragment, or other immune binder, e.g., CD34-binding scFv, CD34-binding CAR, or CD34-binding antibody-drug-conjugate. In some embodiments, the CD34-edited HSCs are not recognized by a CD34-targeted immunotherapeutic, e.g., by a CD34-targeted CAR-T cell or a CD34-targeted CAR-NK cell.

Dual gRNA Compositions and Uses Thereof

In some embodiments, a gRNA provided herein (e.g., a gRNA provided in Table 1 or 2) can be used in combination with a second gRNA, e.g., for targeting a CRISPR/Cas nuclease to two sites in a genome. For instance, in some embodiments it may desired to produce a hematopoietic cell that is deficient for CD34 and a lineage-specific cell surface antigen, e.g., CD33, CD123, CLL-1, CD19, CD30, CD5, CD6, CD7, CD38, or BCMA, so that the cell can be resistant to two agents: an anti-CD34 agent and an agent targeting the lineage-specific cell surface antigen. In some embodiments, the hematopoietic cell is deficient for CD34 and a lymphoid-specific cell surface antigen or a myeloid-specific cell-surface antigen. In some embodiments, it is desirable to contact a cell with two different gRNAs that target different sites of CD34, e.g., in order to make two cuts and create a deletion or an insertion between the two cut sites. Accordingly, the disclosure provides various combinations of gRNAs and related CRISPR systems, as well as cells created by genome editing methods using such combinations of gRNAs and related CRISPR systems. In some embodiments, the CD34 gRNA binds a different nuclease than the second gRNA. For example, in some embodiments, the CD34 gRNA may bind Cas9 and the second gRNA may bind Cas12a, or vice versa.

In some embodiments, the first gRNA is a CD34 gRNA provided herein (e.g., a gRNA provided in Table 1 or 2 or a variant thereof) and the second gRNA targets a lineage-specific cell-surface antigen chosen from: BCMA, CD19, CD20, CD30, ROR1, B7H6, B7H3, CD23, CD33, CD38, C-type lectin like molecule-1, CS1, IL-5, L1-CAM, PSCA, PSMA, CD138, CD133, CD70, CD5, CD6, CD7, CD13, NKG2D, NKG2D ligand, CLEC12A, CD11, CD123, CD56, CD30, CD14, CD66b, CD41, CD61, CD62, CD235a, CD146, CD326, LMP2, CD22, CD52, CD10, CD3/TCR, CD79/BCR, and CD26.

In some embodiments, the first gRNA is a CD34 gRNA provided herein (e.g., a gRNA provided in Table 1 or 2 or a variant thereof) and the second gRNA targets a lymphoid-specific cell-surface antigen (e.g., a T cell-, B Cell-, or NK cell-specific cell-surface antigen). In some embodiments, the lymphoid-specific cell-surface antigen is CD3, CD4, CD8, CD19, CD20, or CD56. In some embodiments, the first gRNA is a CD34 gRNA provided herein (e.g., a gRNA provided in Table 1 or 2 or a variant thereof) and the second gRNA targets a myeloid-specific cell-surface antigen (e.g., a monocyte-, macrophage-, neutrophil-, basophil-, dendritic cell-, erythrocyte-, or platelet-specific cell-surface antigen). In some embodiments, the myeloid-specific cell-surface antigen is CD11c, CD123, CD14, CD33, CD66b, CD41, CD61, CD62, or CD235a. Additional lymphoid- and myeloid-specific cell-surface antigens and the cell types to which they are associated can be found in, e.g., BD Biosciences Human and Mouse CD Marker Handbook, accessible at www.bd.com/documents/bd-legacy/catalogue/biosciences/DS_Human-Mouse-CD-Maker-Biosciences_CT_DE.pdf, last accessed Dec. 30, 2021, the lists of which are incorporated by reference herein.

In some embodiments, the first gRNA is a CD34 gRNA provided herein (e.g., a gRNA provided in Table 1 or 2 or a variant thereof) and the second gRNA targets a lineage-specific cell-surface antigen associated with a neoplastic or malignant disease or disorder, e.g., with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

In some embodiments, the first gRNA is a CD34 gRNA provided herein (e.g., a gRNA provided in Table 1 or 2 or a variant thereof) and the second gRNA targets a lineage-specific cell-surface antigen chosen from: CD1a, CD1b, CD1c, CD1d, CD1e, CD2, CD3, CD3d, CD3c, CD3g, CD4, CD5, CD6, CD7, CD8a, CD8b, CD9, CD10, CD11a, CD11b, CD11c, CD11d, CDw12, CD13, CD14, CD15, CD16, CD16 b, CD17, CD18, CD19, CD20, CD21, CD22, CD23, CD24, CD25, CD26, CD27, CD28, CD29, CD30, CD31, CD32a, CD32b, CD32c, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49c, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300c, CD301, CD302, CD303, CD304, CD305, CD306, CD307a, CD307b, CD307c, CD307d, CD307c, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 or CD363.

In some embodiments, the second gRNA is a gRNA disclosed in any of WO2017/066760, WO2019/046285, WO/2018/160768, or Borot et al. PNAS (2019) 116 (24):11978-11987, each of which is incorporated herein by reference in its entirety.

### Methods of Administration to Subjects in Need Thereof

Some aspects of this disclosure provide methods comprising administering an effective number of genetically engineered cells as described herein, comprising a modification in their genome that results in a loss of expression of CD34, or expression of a variant form of CD34 that is not recognized by an immunotherapeutic agent targeting CD34, to a subject in need thereof.

A subject in need thereof is, in some embodiments, a subject undergoing or about to undergo an immunotherapy targeting CD34. A subject in need thereof is, in some embodiments, a subject having or having been diagnosed with an autoimmune disease, e.g., characterized by detrimental immune activity of CD34-expressing cells. A subject in need thereof is, in some embodiments, a subject having or having been diagnosed with, a malignancy characterized by expression of CD34 on malignant cells. In some embodiments, a subject having such a malignancy or autoimmune disease may be a candidate for immunotherapy targeting CD34, but the risk of detrimental on-target, off-disease effects may outweigh the benefit, expected or observed, to the subject. In some such embodiments, administration of genetically engineered cells as described herein, results in an amelioration of the detrimental on-target, off-disease effects, as the genetically engineered cells provided herein are not targeted efficiently by an immunotherapeutic agent targeting CD34.

Examples of autoimmune diseases for which the cells, compositions, and methods described herein may be useful include, without limitation, Achalasia, Addison's disease, Adult Still's disease, Agammaglobulinemia, Alopecia arcata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner car disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopeniaurpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Parancoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Discasc.

In some embodiments, a subject having such a malignancy or autoimmune disease is a candidate for a radiation therapy, e.g., to ablate malignant cells (e.g., CD34 expressing malignant cells). In some embodiments, the risk of detrimental off-target effects (e.g., to adjacent or surrounding cells or tissue) and on-target off-disease effects (e.g., to non-malignant CD34-expressing cells), may outweigh the benefit, expected or observed, to the subject for radiation therapy. In some embodiments, administration of genetically engineered cells (e.g., genetically engineered hematopoietic cells, e.g., HSCs) described herein after radiation therapy results in an amelioration of the detrimental on-target, off-disease effects. Without wishing to be bound by theory, the combination of an immunotherapeutic approach, e.g., comprising lymphocyte effector cells targeting CD34, such as CAR-T cells or CAR-NK cells, and genetically engineered cells (e.g., genetically engineered stem cells, e.g., HSCs) that do not express CD34, express a reduced level of CD34 (e.g., relative at a wild type cell), or express a variant form of CD34 that is not recognized by an immunotherapeutic agent targeting CD34 is thought to be an alternative to radiation therapy for a subject having a CD34-expressing malignancy or an autoimmune disease characterized by detrimental immune activity of CD34-expressing cells. An immunotherapeutic approach targeting CD34 is thought to avoid or significantly decrease the risk of off-target effects (e.g., to adjacent or surrounding cells or tissue). Replenishment of depleted stem cell populations with immunotherapy-resistant genetically engineered cells (e.g., genetically engineered stem cells, e.g., HSCs) that do not express CD34, express a reduced level of CD34 (e.g., relative at a wild type cell), or express a variant form of CD34 that is not recognized by an immunotherapeutic agent targeting CD34 is thought to ameliorate or eliminate on-target off-disease effects of the immunotherapeutic approach.

In some embodiments, the malignancy is a hematologic malignancy, or a cancer of the blood. In some embodiments, the malignancy is a lymphoid malignancy. In general, lymphoid malignancies are associated with the inappropriate production, development, and/or function of lymphoid cells, such as lymphocytes of the T lineage or the B lineage. In some embodiments, the malignancy is characterized or associated with cells that express CD34 on the cell surface.

In some embodiments, the malignancy is associated with aberrant T lymphocytes, such as a T-lineage cancer, e.g., a T cell leukemia or a T-cell lymphoma.

Examples of T cell leukemias and T-cell lymphomas include, without limitation, T-lineage Acute Lymphoblastic Leukemia (T-ALL), Hodgkin's lymphoma, or a non-Hodgkin's lymphoma, acute lymphoblastic leukemia (ALL), chronic lymphocytic leukemia (CLL), large granular lymphocytic leukemia, adult T-cell leukemia/lymphoma (ATLL), T-cell prolymphocytic leukemia (T-PLL), T-cell chronic lymphocytic leukemia, T-prolymphocytic leukemia, T-cell lymphocytic leukemia, peripheral T-cell lymphoma not otherwise specified (PTCL-NOS), enteropathy associated T-cell lymphoma, B-cell chronic lymphocytic leukemia, mantle cell lymphoma, peripheral T-cell lymphoma (PTCL), anaplastic large-cell lymphoma, cutaneous T-cell lymphoma, angioimmunoblastic lymphoma, anaplastic large cell lymphoma, enteropathy-type T-cell lymphoma, hematosplenic gamma-delta T-cell lymphoma, lymphoblastic lymphoma, or hairy cell leukemia.

In some embodiments, the malignancy is associated with aberrant B lymphocytes, such as a B-lineage cancer, e.g., a B-cell leukemia or a B-cell lymphoma. In some embodiments, the malignancy is B-lineage Acute Lymphoblastic Leukemia (B-ALL) or chronic lymphocytic leukemia (B-CLL), primary mediastinal B-cell lymphoma.

In some embodiments, cells of the malignancy express CD34, e.g., on their surfaces.

In some embodiments, the malignancy comprises a population of cells characterized by expression of CD34. In some embodiments, the population of cells characterized by expression of CD34 are cancer stem cells. Without wishing to be bound by theory, the cancer stem cell theory suggests that for some malignancies, cancer stem cells share many properties with normal healthy stem cells. In some embodiments, a cancer stem cell expresses CD34, e.g., on its surface. In some embodiments, an immunotherapeutic approach described herein, e.g., comprising lymphocyte effector cells targeting CD34, such as CAR-T cells or CAR-NK cells, specifically targets the cancer stem cells of a malignancy. In some embodiments, an immunotherapeutic approach described herein that targets cancer stem cells also has detrimental on-target off-disease effects, e.g., on healthy stem cells. In some embodiments, genetically engineered cells (e.g., genetically engineered stem cells, e.g., HSCs) that do not express CD34, express a reduced level of CD34 (e.g., relative at a wild type cell), or express a variant form of CD34 that is not recognized by an immunotherapeutic agent targeting CD34 are used to replenish or replace non-cancer stem cells (e.g., healthy stem cells) targeted by the immunotherapeutic approach.

In some embodiments, the malignancy is graft-versus host disease.

Also within the scope of the present disclosure are malignancies that are considered to be relapsed and/or refractory, such as relapsed or refractory hematological malignancies. A subject in need thereof is, in some embodiments, a subject undergoing or that will undergo an immune effector cell therapy targeting CD34, e.g., CAR-T cell therapy, wherein the immune effector cells express a CAR targeting CD34, and wherein at least a subset of the immune effector cells also express CD34 on their cell surface or healthy stem cells (e.g., HSCs) in the subject undergoing the therapy express CD34 on their cell surface. As used herein, the term “fratricide” refers to self-killing. For example, cells of a population of cells kill or induce killing of cells of the same population. In some embodiments, cells of the immune effector cell therapy kill or induce killing of other cells of the immune effector cell therapy. In such embodiments, fratricide ablates a portion of or the entire population of immune effector cells before a desired clinical outcome, e.g., ablation of malignant cells expressing CD34 within the subject, can be achieved. In some such embodiments, using genetically engineered immune effector cells, as provided herein, e.g., immune effector cells that do not express CD34 or do not express a CD34 variant recognized by the CAR, as the immune effector cells forming the basis of the immune effector cell therapy, will avoid such fratricide and the associated negative impact on therapy outcome. In such embodiments, genetically engineered immune effector cells, as provided herein, e.g., immune effector cells that do not express CD34 or do not express a CD34 variant recognized by the CAR, may be further modified to also express the CD34-targeting CAR. In some embodiments, the immune effector cells may be lymphocytes, e.g., T-lymphocytes, such as, for example alpha/beta T-lymphocytes, gamma/delta T-lymphocytes, or natural killer T cells. In some embodiments, the immune effector cells may be natural killer (NK) cells.

In some embodiments, cells of the immune effector cell therapy kill or induce killing of stem cells (e.g., HSCs) expressing CD34 on their cell surface in the subject. In some embodiments, methods described herein result in depletion of a target stem cell niche (e.g., an HSC niche) in a subject. In some embodiments, methods described herein do not alter or do not appreciably alter the level or viability of stem cells in at least one non-target stem cell niche in a subject. In some embodiments, methods described herein target all stem cell niches of a particular type in a subject (e.g., all HSC niches). In some embodiments, methods described herein result in complete depletion of a stem cell niche (e.g., an HSC niche) in a subject. As used herein, a stem cell niche refers to an anatomical area of a subject comprising a specific microenvironment comprising a population of stem cells in an undifferentiated and self-renewable state.

In some embodiments, administering to the subject genetically engineered stem cells not expressing CD34 or expressing a variant form of CD34 that is not recognized by an immunotherapeutic agent targeting CD34 replenishes the supply of stem cells (e.g., HSCs) in the subject. In some embodiments, a subject is administered genetically engineered stem cells not expressing CD34 or expressing a variant form of CD34 that is not recognized by an immunotherapeutic agent targeting CD34 in combination with immune effector cells targeting CD34 (e.g., genetically engineered immune effector cells as provided herein, e.g., immune effector cells that do not express CD34 or do not express a CD34 variant recognized by the CAR, which may be further modified to also express the CD34-targeting CAR).

In some embodiments, an effective number of genetically engineered cells as described herein, comprising a modification in their genome that results in a loss of expression of CD34, or expression of a variant form of CD34 that is not recognized by an immunotherapeutic agent targeting CD34, is administered to a subject in need thereof, e.g., to a subject undergoing or that will undergo an immunotherapy targeting CD34, wherein the immunotherapy is associated or is at risk of being associated with a detrimental on-target, off-disease effect, e.g., in the form of cytotoxicity towards healthy cells in the subject that express CD34. In some embodiments, an effective number of such genetically engineered cells may be administered to the subject in combination with the anti-CD34 immunotherapeutic agent.

It is understood that when agents (e.g., CD34-modified cells and an anti-CD34 immunotherapeutic agent) are administered in combination, the cells and the agent may be administered at the same time or at different times, e.g., in temporal proximity. Furthermore, the cells and the agent may be admixed or in separate volumes or dosage forms. For example, in some embodiments, administration in combination includes administration in the same course of treatment, e.g., in the course of treating a subject with an anti-CD34 immunotherapy, the subject may be administered an effective number of genetically engineered, CD34-modified cells concurrently or sequentially, e.g., before, during, or after the treatment, with the anti-CD34 immunotherapy.

In some embodiments, the immunotherapeutic agent that targets CD34 as described herein is an immune cell that expresses a chimeric antigen receptor, which comprises an antigen-binding fragment (e.g., a single-chain antibody) capable of binding to CD34. The immune cell may be, e.g., a T cell (e.g., a CD4+ or CD8+ T cell) or an NK cell.

A Chimeric Antigen Receptor (CAR) can comprise a recombinant polypeptide comprising at least an extracellular antigen binding domain, a transmembrane domain, and a cytoplasmic signaling domain comprising a functional signaling domain, e.g., one derived from a stimulatory molecule. In one some embodiments, the cytoplasmic signaling domain further comprises one or more functional signaling domains derived from at least one costimulatory molecule, such as 4-1BB (i.e., CD137), CD27, and/or CD28, or fragments of those molecules. The extracellular antigen binding domain of the CAR may comprise a CD34-binding antibody fragment. The antibody fragment can comprise one or more CDRs, the variable regions (or portions thereof), the constant regions (or portions thereof), or combinations of any of the foregoing.

A chimeric antigen receptor (CAR) typically comprises an antigen-binding domain, e.g., comprising an antibody fragment, fused to a CAR framework, which may comprise a hinge region (e.g., from CD8 or CD28), a transmembrane domain (e.g., from CD8 or CD28), one or more costimulatory domains (e.g., CD28 or 4-1BB), and a signaling domain (e.g., CD3zeta). Exemplary sequences of CAR domains and components are provided, for example in PCT Publication No. WO 2019/178382, and in Table 3 below.

TABLE 3
Exemplary components of a chimeric antigen receptor

| Chimeric receptor component | Amino acid sequence |
|---|---|
| Antigen-binding fragment | e.g., scFv, light chain-linker- |
| | heavy chain, single- |
| | domain antibody, or nanobody |
| CD28 costimulatory domain | IEVMYPPPYLDNEKSNGTIIHVKGKHLCP |
| | SPLFPGPSKPFWVLVVVGGVLACYSLLVTV |
| | AFIIFWVRSKRSRLLHSDYMNMTPRRPGPT |
| | RKHYQPYAPPRDFAAYRS (SEQ ID NO: 19) |
| CD8alpha transmembrane | IYIWAPLAGTCGVLLLSLVITLYC |
| domain | (SEQ ID NO: 20) |
| CD28 transmembrane domain | FWVLVVVGGVLACYSLLVTVAFII |
| | FWVRSKRSRLLHSDYMNMTPRR |
| | PGPTRKHYQPYAPPRDFAAYRS |
| | (SEQ ID NO: 21) |
| 4-1BB intracellular domain | KRGRKKLLYIFKQPFMRVQTTQEEDGCS |
| | CRFPEEEEGGCEL (SEQ ID NO: 22) |
| CD3ζ cytoplasmic signaling | RVKFSRSADAPAYQQGQNQLYNELNLGR |
| domain | REEYDVLDKRRGRDPEMGGKPQRRKNPQ |
| | EGLYNELQKDKMAEAYSEIGMKGERRRG |
| | KGHDGLYQGLSTATKDTYDALHMQALPP |
| | R (SEQ ID NO: 23) |

In some embodiments, the number of genetically engineered cells provided herein, e.g., HSCs, HPCs, or immune effector cells that are administered to a subject in need thereof, is within the range of 10⁶-10¹¹. However, amounts below or above this exemplary range are also within the scope of the present disclosure. For example, in some embodiments, the number of genetically engineered cells provided herein, e.g., HSCs, HPCs, or immune effector cells that are administered to a subject in need thereof is about 10⁶, about 10⁷, about 10⁸, about 10⁹, about 10¹⁰, or about 10¹¹. In some embodiments, the number of genetically engineered cells provided herein, e.g., HSCs, HPCs, or immune effector cells that are administered to a subject in need thereof, is within the range of 10⁶-10⁹, within the range of 10⁶-10⁸, within the range of 10⁷-10⁹, within the range of about 10⁷-10¹⁰, within the range of 10⁸-10¹⁰, or within the range of 10⁹-10¹¹.

In some embodiments, the immunotherapeutic agent that targets CD34 is an antibody-drug conjugate (ADC). The ADC may be a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on the its cell surface (e.g., target cell), thereby resulting in death of the target cell.

Suitable antibodies and antibody fragments binding CD34 that can be used for the generation of immunotherapeutics, for example, of chimeric antigen receptors, of CAR-T cells expressing such chimeric receptors, or for the generation of antibody-drug-conjugates, will be apparent to those of ordinary skill in the art. Examples of suitable anti-CD34 antibodies include, without limitation, EP373Y (Abcam (rabbit, unconjugated)), clone QBEnd-10 (LifeSpan BioSciences (mouse)), clone MEC14.7 (LifeSpan BioSciences (rat)), clone SI16-01 (antibody name ET1606-11; HUABIO (rabbit), clone HPCA1/2598R (biorbyt (rabbit), 4C8 and humanized h4C8, 4H11, cQBEND/10 or hQBEND/10 variants of QBEnd-10, 27H2, CIRMA-K4, DS554AB, 10C304, 9C5, B-C34, RM300, AC136.

Any suitable antibodies and antigen-binding fragments capable of binding human CD34 (mRNA NCBI Reference Sequence: NM_001025109.1, Protein NCBI Reference Sequence: NP_001020280.1) can be used in conjunction with the compositions and methods described herein. There are two isoforms of CD34 that differ in the length of their cytoplasmic tail (long and short). Recently, the long isoform was used to generate a stable cell line expressing CD34 that could be used as an immunogen (see, e.g., Adv. Pharm. Bull. 5:69-75, 2015). CD34, such as the long isoform of CD34, can be used as an immunogen in order to identify antibodies and antigen-binding fragments thereof capable of binding CD34 and to generate suitable CD34-targeted immunotherapeutics, for example, CD34-targeted CARs and CAR-T cells, or antibody-drug-conjugates, for the treatment of cancers and autoimmune diseases, as well as for use as a conditioning agent prior to hematopoietic stem cell transplant therapy.

Some suitable CD34 antibodies, CD34-binding antibody fragments, and CD34-binding scFvs are described herein and additional suitable CD34 antibodies, CD34-binding antibody fragments, and CD34-binding scFvs will be apparent to the skilled artisan based on the present disclosure. These include, without limitation, CD34-binding antibodies produced and released from ATCC Accession No. AC133.1 and HB 12346, as described, for example, in U.S. Pat. No. 5,843,633, incorporated herein by reference.

Suitable CD34-binding antibodies and CD34-binding antibody fragments and scFvs that may be used in conjunction with the compositions and methods described herein further include humanized variants of CD34 antibodies, CD34-binding antibody fragments, scFvs, as well as any antibodies, antibody fragments, and scFvs that specifically bind the same CD34 epitope as those described herein, as assessed, for instance, by way of a competitive CD34 binding assay.

Toxins or drugs compatible for use in antibody-drug conjugates are known in the art and will be evident to one of ordinary skill in the art. See, e.g., Peters et al. Biosci. Rep. (2015) 35(4): e00225; Beck et al. Nature Reviews Drug Discovery (2017) 16:315-337; Marin-Acevedo et al. J. Hematol. Oncol. (2018)11:8; Elgundi et al. Advanced Drug Delivery Reviews (2017) 122: 2-19.

In some embodiments, the antibody-drug conjugate may further comprise a linker (e.g., a peptide linker, such as a cleavable linker) attaching the antibody and drug molecule.

Examples of suitable toxins or drugs for antibody-drug conjugates include, without limitation, the toxins and drugs comprised in brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Ax1-ADC, pinatuzumab vedotin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD33 A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861.

In some embodiments, binding of the antibody-drug conjugate to the epitope of the cell-surface lineage-specific protein induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.): Handbook of Experimental Immunology (D. M. Weir and C. C. Blackwell, eds.): Gene Transfer Vectors for Mammalian Cells (J. M. Miller and M. P. Calos, eds., 1987; Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, cd. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1: Genetic Editing of CD34 in Human Cells

Design of sgRNA Constructs

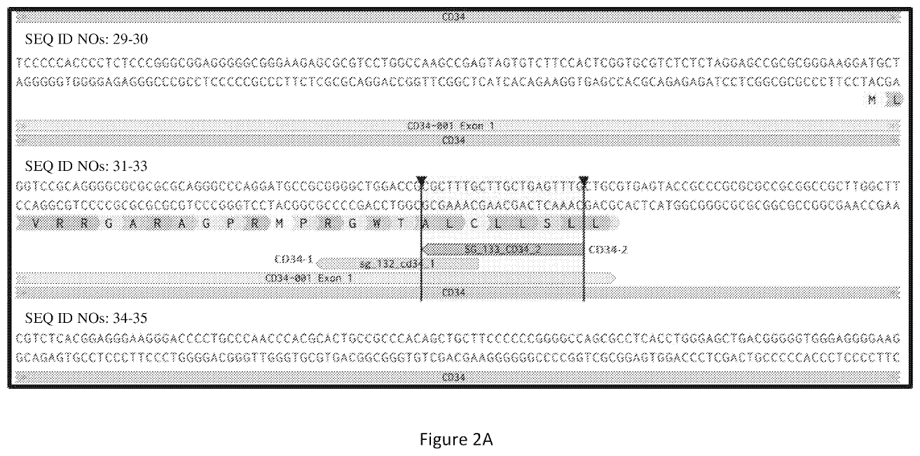

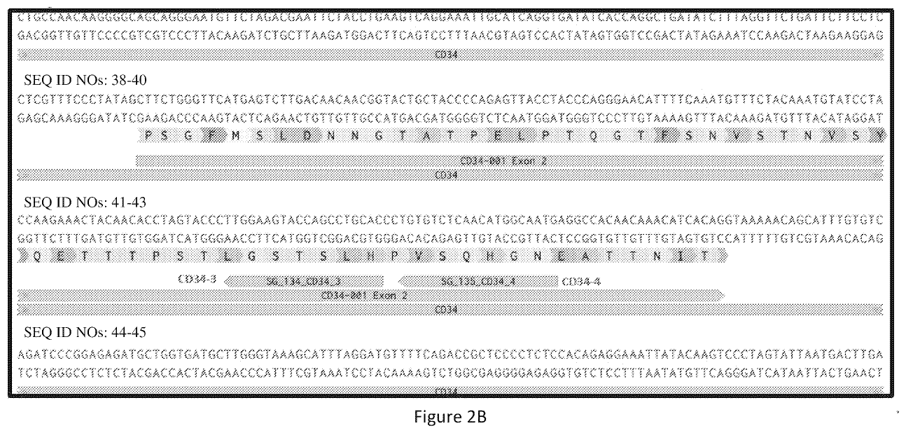

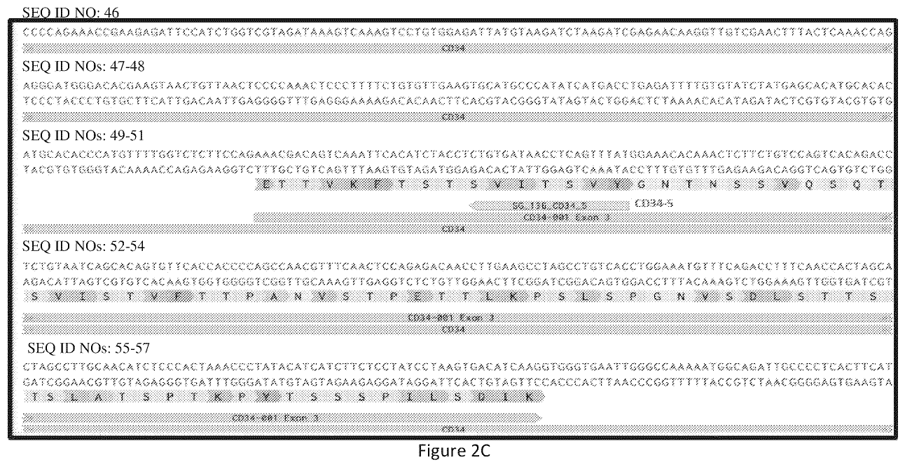

The target domains and gRNAs indicated in Tables 1 and 2 were designed by manual inspection for a PAM sequence for an applicable nuclease, e.g., Cas9, with close proximity to the target region and prioritized according to predicted specificity by minimizing potential off-target sites in the human genome with an online search algorithm (e.g., the Benchling algorithm, Doench et al 2016, Hsu et al 2013). FIGS. 1 and 2A-2C show the location of exemplary gRNAs within the CD34 gene. All designed synthetic sgRNAs were produced with chemically modified nucleotides at the three terminal positions at both the 5′ and 3′ ends. Modified nucleotides contained 2′-O-methyl-3′-phosphorothioate (abbreviated as “ms”) and the ms-sgRNAs were HPLC-purified.

### Editing Human CD34+ HSCs

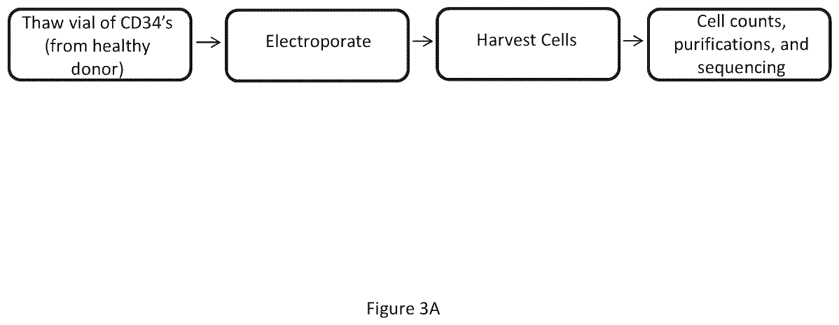

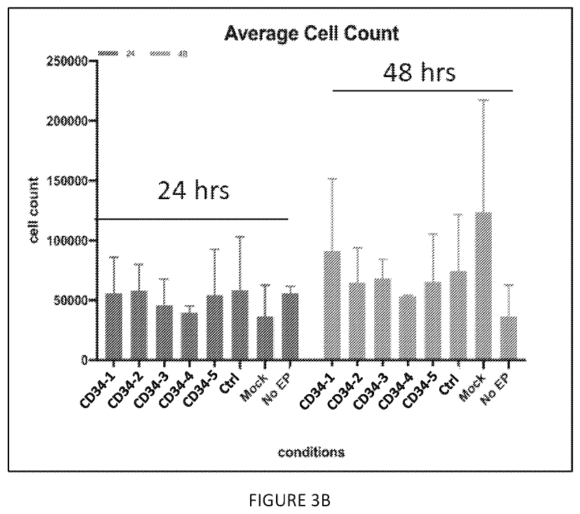

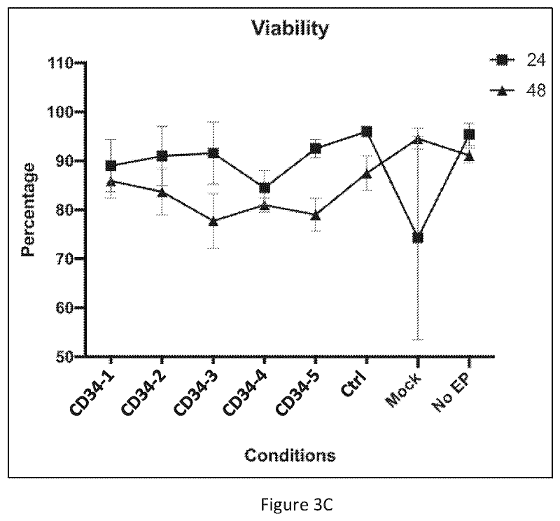

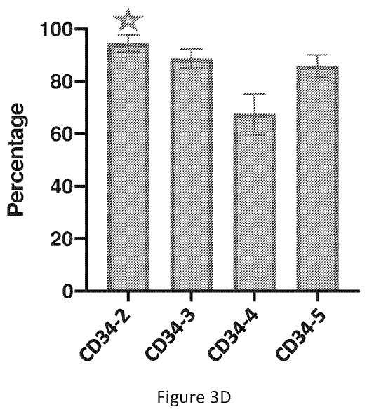

Frozen CD34+ HSCs were thawed according to manufacturer's instructions. To edit HSCs, ˜HSCs were thawed and cultured in StemSpan SFEM medium supplemented with StemSpan CC110 cocktail (StemCell Technologies) for approximately 48 h before electroporation with RNP, as shown in FIG. 3A. To electroporate HSCs, approximately 2×10⁵ cells were pelleted and resuspended in electroporation solution and mixed with 3 μg Cas9 and 3 μg gRNA in the form of RNP. CD34+ HSCs were electroporated using standard electroporation procedures. Experimental conditions are provided in Table 4.

TABLE 4

| Number of | | | Guide |
|---|---|---|---|
| cells | Conditions | Cas9 | concentration |
| 200K | mock electroporated | 0 | 0 |
| | (Mock) | | |
| 200K | positive control | 3 ug | 3 ug |
| 200K | Cas9 + gRNA CD34-1 | 3 ug | 3 ug |
| 200K | Cas9 + gRNA CD34-2 | 3 ug | 3 ug |
| 200K | Cas9 + gRNA CD34-3 | 3 ug | 3 ug |
| 200K | Cas9 + gRNA CD34-4 | 3 ug | 3 ug |
| 200K | Cas9 + gRNA CD34-5 | 3 ug | 3 ug |
| 200K | Cas9 + gRNA CD34-1 | 3 ug | 3 ug |
| 200K | Cas9 + gRNA CD34-2 | 3 ug | 3 ug |
| 200K | Cas9 + gRNA CD34-3 | 3 ug | 3 ug |
| 200K | Cas9 + gRNA CD34-4 | 3 ug | 3 ug |
| 200K | Cas9 + gRNA CD34-5 | 3 ug | 3 ug |
| 200K | no electroporation (No | 0 | 0 |
| | EP) | | |

### Genomic DNA Analysis

Approximately 48 hours following electroporation, cells were harvested for cell counts, viability analysis, purification, and sequencing. For all genomic analysis, DNA was harvested from cells, amplified with primers flanking the target region, purified and the allele modification frequencies were analyzed using TIDE (Tracking of INDELs by Decomposition). Analyses were performed using a reference sequence from a mock-transfected sample.

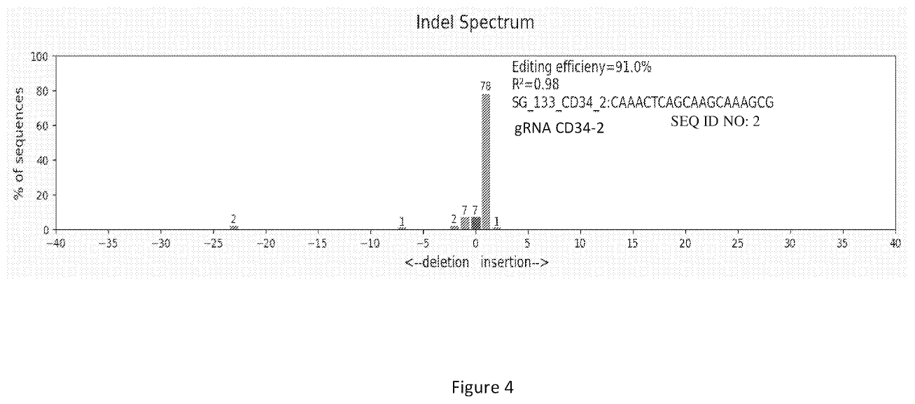

Human CD34+ cells were electroporated with Cas9 protein and indicated CD34-targeting gRNAs, as described above. The percentage editing was determined by % INDEL as assessed by TIDE analysis. Editing efficiency was determined by TIDE analysis, as shown in FIGS. 3D and 4.

### Flow Cytometry Analysis

The CD34 gRNA-edited cells may also be evaluated for surface expression of CD34 protein, for example by flow cytometry analysis (FACS). Live HSCs are stained for CD34 using an anti-CD34 antibody and analyzed by flow cytometry on the Attune NxT flow cytometer (Life Technologies). Cells in which the CD34 gene have been genetically modified show a reduction in CD34 expression as detected by FACS.

### Viability of Edited Cells

At 24 hours and 48 hours post-ex vivo editing, the average cell counts and percentages of viable, edited CD34KO cells and control cells were quantified using flow cytometry and the 7AAD viability dye. As shown in FIGS. 3B and 3C, CD34KO cells edited using the CD34 gRNAs described herein were viable and remain viable over time following electroporation and gene editing, similar to what is observed in the control cells editing with a gRNA targeting CD5, mock edited cells, and cells that were not electroporated.

### Example 2: Large Scale Electroporation-96 hr Time Point

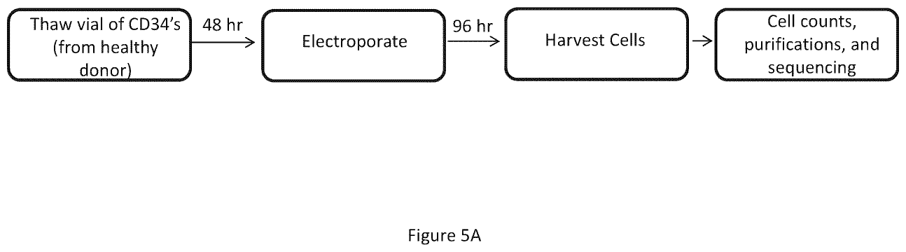

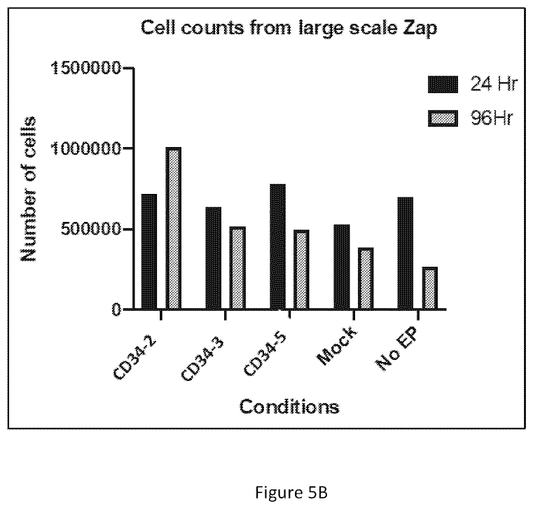

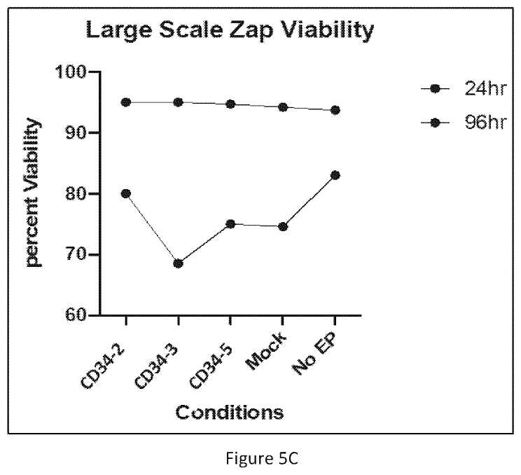

Frozen CD34+ HSCs were thawed according to manufacturer's instructions. To edit HSCs, ˜HSCs were thawed and cultured in StemSpan SFEM medium supplemented with StemSpan CC110 cocktail (StemCell Technologies) for approximately 48 h before electroporation with RNP, as shown in FIG. 5A. To electroporate HSCs, approximately 1×10⁶ cells were pelleted and resuspended in electroporation solution and mixed with 15 μg Cas9 and 15 μg gRNA in the form of RNP. CD34+ HSCs were electroporated using standard electroporation procedures. Experimental conditions are provided in Table 5.

TABLE 5

| Number | | | Guide |
|---|---|---|---|
| of cells | Conditions | Cas9 | concentration |
| 1M | Cas9 + gRNA CD34-2 | 15 ug | 15 ug |
| 1M | Cas9 + gRNA CD34-3 | 15 ug | 15 ug |
| 1M | Cas9 + gRNA CD34-5 | 15 ug | 15 ug |
| 1M | Mock electroporated | | |
| | (Mock) | | |
| 1M | No electroporation (No | | |
| | EP) | | |

At 24 hours and 96 hours post electroporation, cells were harvested for cell counts, viability analysis, purification, and sequencing analysis. Cell counts and viability of cells edited using the indicated CD34 gRNAs were found to have similar levels of viability as mock edited cells (Mock) or cells that were not electroporated (No EP), as shown in FIGS. 5B and 5C.

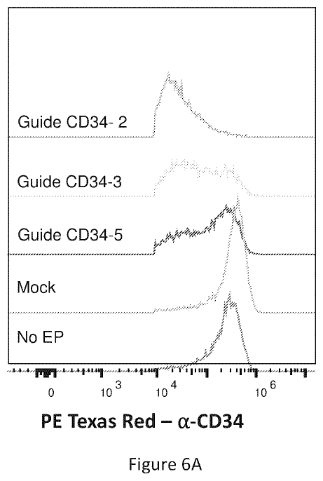

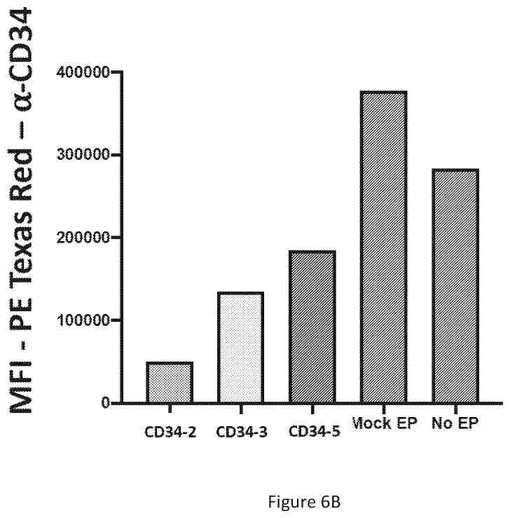

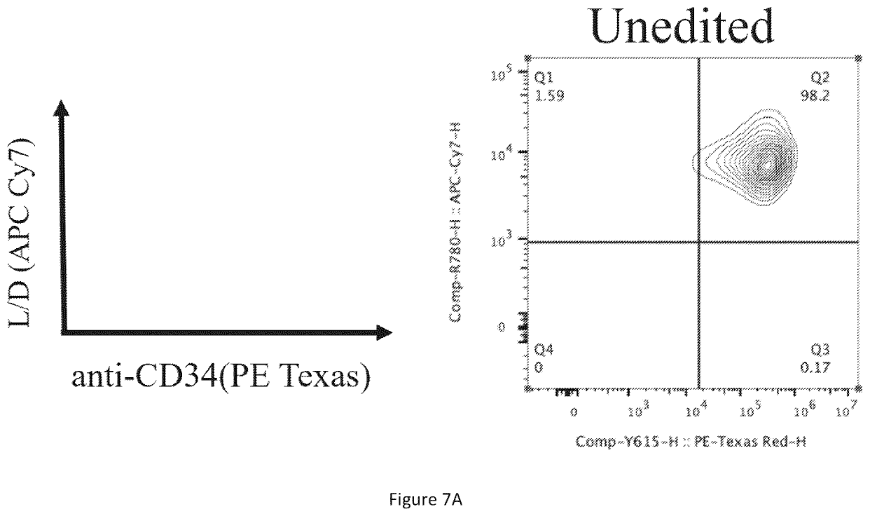

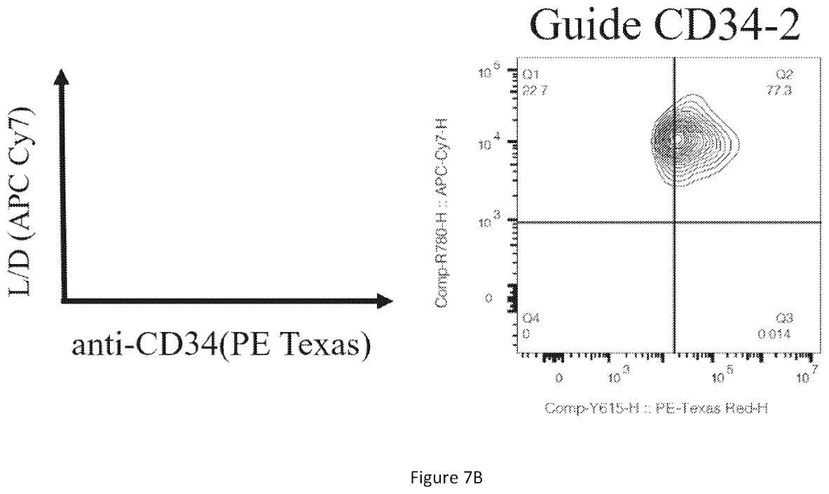

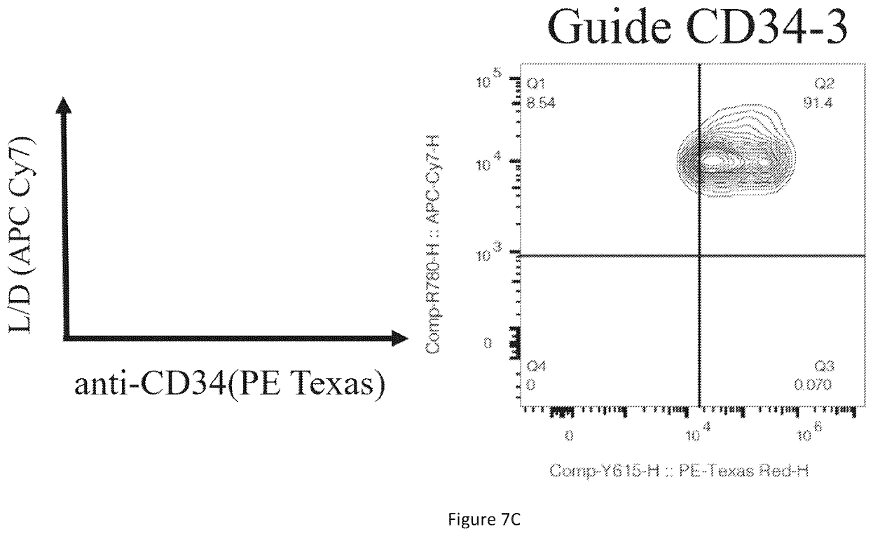

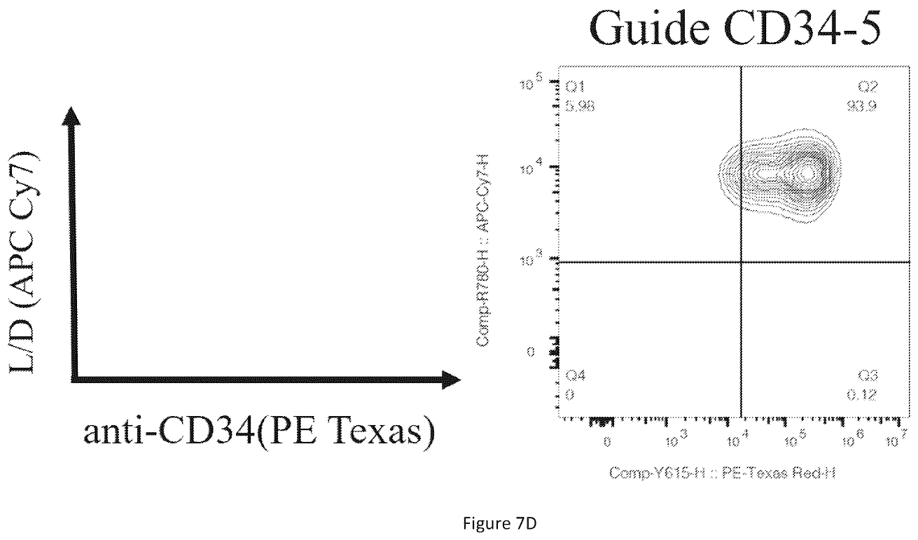

The CD34 gRNA-edited cells were also be evaluated for surface expression of CD34 protein by flow cytometry analysis (FACS) using an anti-CD34 antibody (anti-CD34-PE Texas Red). Cells in which the CD34 gene have been genetically modified were found to have reduced CD34 expression as detected by FACS. See, FIGS. 6A, 6B, and 7A-7D.

### Example 3: Large Scale Electroporation-168 Hour Time Point

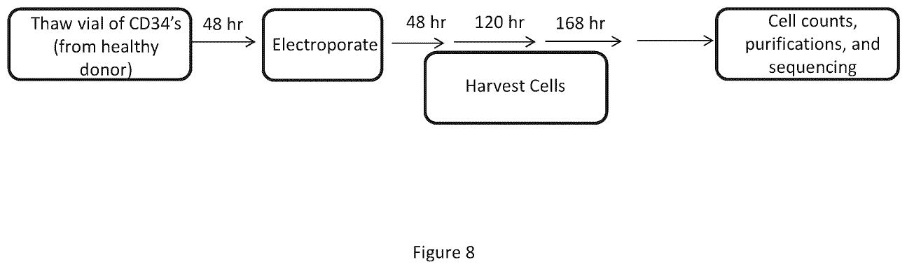

Frozen CD34+ HSCs were thawed according to manufacturer's instructions. To edit HSCs, ˜HSCs were thawed and cultured in StemSpan SFEM medium supplemented with StemSpan CC110 cocktail (StemCell Technologies) for approximately 48 h before electroporation with RNP, as shown in FIG. 8. To electroporate HSCs, approximately 1×10⁶ cells were pelleted and resuspended in electroporation solution and mixed with 15 μg Cas9 and 15 μg gRNA in the form of RNP, and then electroporated using standard electroporation procedures. Experimental conditions are provided in Table 6.

TABLE 6

| Number of | | | Guide |
|---|---|---|---|
| cells | Conditions | Cas9 | concentration |
| 1M | Cas9 + gRNA CD34-2 | 15 ug | 15 ug |
| 1M | Cas9 + gRNA CD34-3 | 15 ug | 15 ug |
| 1M | Mock electroporated | | |
| | (Mock) | | |
| 1M | No electroporation (No | | |
| | EP) | | |

At 24, 48, 120, 144, and 168 hours post electroporation, cells were harvested for cell counts, viability analysis, purification, and sequencing analysis. Cell counts and viability of cells edited using the indicated CD34 gRNAs were found to have similar levels of viability as mock edited cells (Mock) or cells that were not electroporated (No EP).

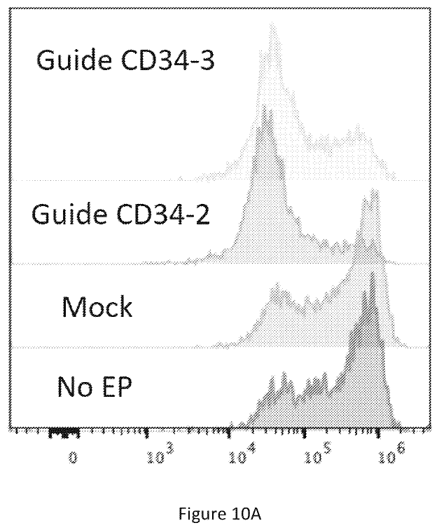

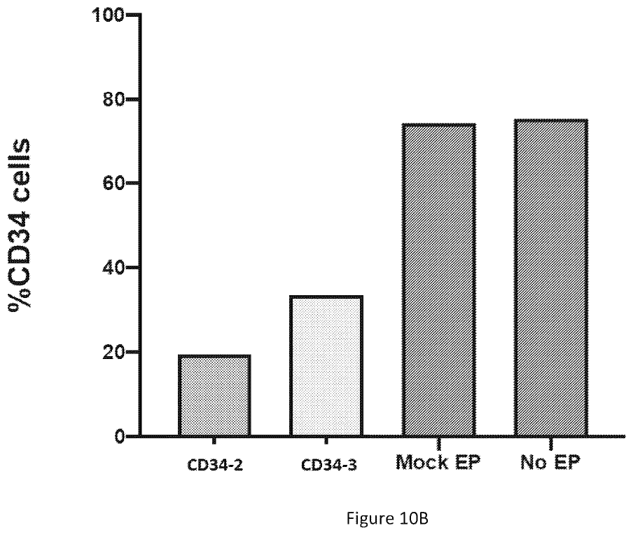

The CD34 gRNA-edited cells were also be evaluated for surface expression of CD34 protein by flow cytometry analysis (FACS) using an anti-CD34 antibody (anti-CD34-PE Texas Red). At 48 hours post electroporation, cells in which the CD34 gene have been genetically modified were found to slightly have reduced CD34 expression as detected by FACS. See, FIGS. 9A and 9B. However, at 168 hours post electroporation, cells in which the CD34 gene have been genetically modified were found to have reduced CD34 expression as detected by FACS. See, FIGS. 10A and 10B.

### Example 4: CAR-T Cytotoxicity Assay

Genetically modified cells produced using the gRNAs shown in Tables 1 or 2 may be evaluated for killing by CD34-CAR T cells.

### CAR Constructs and Lentiviral Production

Second-generation CARs are constructed to target CD34. An exemplary CAR construct consists of an extracellular scFv antigen-binding domain, using CD8α signal peptide, CD8α hinge and transmembrane regions, the 4-1BB costimulatory domain, and the CD3ξ signaling domain. The anti-CD34 scFv sequence may be obtained from any anti-CD34 antibody known in the art. CAR cDNA sequences for the target are sub-cloned into the multiple cloning site of the pCDH-EF1α-MCS-T2A-GFP expression vector, and lentivirus is generated following the manufacturer's protocol (System Biosciences). Lentivirus can be generated by transient transfection of 293TN cells (System Biosciences) using Lipofectamine 3000 (ThermoFisher). The exemplary CAR construct is generated by cloning the light and heavy chain of an anti-CD34 antibody, to the CD8α hinge domain, the ICOS transmembrane domain, the ICOS signaling domain, the 4-1BB signaling domain and the CD3ξ signaling domain into the lentiviral plasmid pHIV-Zsgreen.

### CAR Transduction and Expansion

Human primary T cells are isolated from Leuko Pak (Stem Cell Technologies) by magnetic bead separation using anti-CD4 and anti-CD8 microbeads according to the manufacturer's protocol (Stem Cell Technologies). Purified CD4+ and CD8+ T cells are mixed 1:1 and activated using anti-CD3/CD28 coupled Dynabeads (Thermo Fisher) at a 1:1 bead to cell ratio. T cell culture media used is CTS Optimizer T cell expansion media supplemented with immune cell serum replacement, L-Glutamine and GlutaMAX (all purchased from Thermo Fisher) and 100 IU/mL of IL-2 (Peprotech). T cell transduction is performed 24 hours post activation by spinoculation in the presence of polybrene (Sigma). CAR-T cells are cultured for 9 days prior to cryopreservation. Prior to all experiments, T cells are thawed and rested at 37° C. for 4-6 hours.

### Flow Cytometry Based CAR-T Cytotoxicity Assay

The cytotoxicity of target cells is measured by comparing survival of target cells relative to the survival of negative control cells. For CD34 cytotoxicity assays, CD34+cells may be used as target cells and CD34+ cells deficient in CD34 or having reduced expression of CD34 may be generated as described in Examples 1-3.

Target cells and negative control cells are stained with CellTrace Violet (CTV) and CFSE (Thermo Fisher), respectively, according to the manufacturer's instructions. After staining, target cells and negative control cells are mixed at 1:1.

Anti-CD34 CAR-T cells are used as effector T cells. Non-transduced T cells (mock CAR-T) are used as control. The effector T cells are co-cultured with the target cell/negative control cell mixture at a 1:1 effector to target ratio in duplicate. A group of target cell/negative control cell mixture alone without effector T cells is included as control. Cells are incubated at 37° C. for 24 hours before flow cytometric analysis. Propidium iodide (ThermoFisher) is used as a viability dye. For the calculation of specific cell lysis, the fraction of live target cell to live negative control cell (termed target fraction) is used. Specific cell lysis is calculated as ((target fraction without effector cells-target fraction with effector cells)/(target fraction without effectors))×100%.

### Example 5: Effect of Anti-CD34 Antibody Drug Conjugates on Engineered HSCs

Genetically modified cells produced using the gRNAs shown in Tables X may be evaluated for killing by antibody-drug conjugates, such as an anti-CD34 antibody to an immunotoxin.

Frozen CD34+ HSPCs derived from mobilized peripheral blood are thawed and cultured for 72 h before electroporation with ribonucleoprotein comprising Cas9 and an sgRNA. Samples are electroporated with the following conditions:
i. Mock (Cas9 only), and
ii. KO sgRNA (such as any one of the CD34 gRNAs shown in Tables X).
   Cells are allowed to recover for 72 hours and genomic DNA is collected and analyzed. The percentage of CD34-positive cells is assessed by flow cytometry to confirm that editing with the CD34 gRNAs is effective in knocking out or reducing CD34 expression. The editing events in the HSCs result in a variety of INDEL sequences.

### Sensitivity of Cells Having CD34 Deletion to Antibody-Drug Conjugates

To determine in vitro toxicity, cells are incubated with the antibody-drug conjugate in the culture media and the number of viable cells is quantified over time. Engineered cells that are deficient in CD34 or have reduced CD34 expression generated with the CD34 gRNAs described herein are more resistant to antibody-drug conjugate treatment than cells expressing full length CD34 (mock).

### (ii) Enrichment of CD34-Edited Cells

To assay if CD34-modified cells are enriched following treatment with the antibody-drug conjugate, HSPCs are edited with 50% of standard nuclease (e.g., Cas9, Cpf1) to gRNA ratios. The bulk population of cells are analyzed prior to and after treatment with the antibody-drug conjugate. Following treatment with the antibody-drug conjugate, CD34-edited cells are enriched so that the percentage of CD34-deficient cells increased.

(iii) In Vitro Differentiation of CD34-Edited HSPCs

Cell populations are assessed for lymphoid differentiation prior to and after treatment with the antibody-drug conjugate at various days post differentiation. Engineered CD34 knockout cells generated with the CD34 gRNAs described herein may show increased expression of differentiation markers, whereas cells expressing full length CD34 (mock) may not differentiate.

### Example 6: Evaluation of the Persistence of CD34KO Cells In Vivo

### Editing in Mobilized Peripheral Blood CD34+ HSCs (mPB CD34+ HSPCs)

gRNAs (Synthego) were designed as described in Examples 1-3. mPB CD34+ HSPCs are purchased from Fred Hutchinson Cancer Center and thawed according to manufacturer's instructions. These cells are then edited via CRISPR/Cas9 as described in Examples 1-3 using the CD34-targeting gRNAs described herein, as well as a non-CD34 targeting control gRNA (gCtrl) that is designed not to target any region in the human or mouse genomes.

At 4, 24, and 48 hours post-ex vivo editing, the percentages of viable, edited CD34KO cells and control cells are quantified using flow cytometry and the 7AAD viability dye. High levels of CD34KO cells edited using the CD34 gRNAs described herein may be viable and remain viable over time following electroporation and gene editing, comparable to what is observed in the control cells edited with the non-CD34 targeting control gRNA, gCtrl.

Additionally, at 48 hours post-ex vivo editing, the genomic DNA is harvested from cells, PCR amplified with primers flanking the target region, purified, and analyzed by TIDE, in order to determine the percentage editing as assessed by INDEL (insertion/deletion), as described in Examples 1-3.

Following TIDE analysis, the percentage of long term-HSCs (LT-HSCs) following editing with the CD34 gRNAs described herein are quantified by flow cytometry. The percentages of LT-HSCs following editing with the specified CD34 gRNAs is assessed. This assay may be performed, for example, at the time of cryopreservation of the edited cells, prior to injection into mice for investigation of persistence of CD34KO cells in vivo. The edited cells are cryopreserved in CS10 media (Stem Cell Technology) at 5×10⁶ cells/mL, in a 1 mL volume of media per vial.

### Investigating Engraftment Efficiency and Persistence of CD34KO mPB HSPCs In Vivo

Female NSG mice (JAX) that are 6 to 8 weeks of age, are allowed to acclimate for 2-7 days. Following acclimation, mice are irradiated using 175 cGy whole body irradiation by X-ray irradiator. This is regarded as day 0 of the investigation. At 4-10 hours, following irradiation, the mice are engrafted with the CD34KO cells generated during any of the CD34 gRNAs described herein or control cells edited with gCtrl. The cryopreserved cells are thawed and counted using a BioRad TC-20 automated cell counter. The number of viable cells is quantified in the thawed vials, which is used to prepare the total number of cells for engraftment in the mice. Mice are given a single intravenous injection of 1×10⁶ edited cells in a 100 μL volume. Body weight and clinical observations are recorded once weekly for each mouse in the four groups.

At weeks 8 and 12 following engraftment, 50 μL of blood is collected from each mouse by retroorbital bleed for analysis by flow cytometry. At week 16, following engraftment, mice are sacrificed, and blood, spleens, and bone marrow are collected for analysis by flow cytometry. Bone marrow is isolated from the femur and the tibia. Bone marrow from the femur is also used for on-target editing analysis. Flow cytometry is performed using the FACSCanto™ 10 color and BDFACSDiva™ software. Cells are generally first sorted by viability using the 7AAD viability dye (live/dead analysis), then Live cells are gated by expression of human CD45 (hCD45) but not mouse CD45 (mCD45). The cells that are hCD45+ are then further gated for the expression of human CD19 (hCD19) (lymphoid cells, specifically B cells). Cells expressing human CD45 (hCD45) are also gated and analyzed for the presence of for various cellular markers of the myeloid lineage.

Numbers of cells expressing each of the analyzed markers that are comparable across all mice regardless of which edited cells they are engrafted with indicates successful engraftment of CD34KO cells edited with the CD34 gRNAs in the blood of mice.

At weeks 8, 12, and 16 following engraftment, the percentage of nucleated blood cells that are hCD45+ is quantified in the groups of mice (n=15 mice/group) that received control cells edited with the control gRNA (gCtrl), or the CD34KO cells. This is quantified by dividing the hCD45+ absolute cell count by the mouse CD45+ (mCD45) absolute cell count.

The percentage of hCD34+ cells in the blood is also quantified at week 8 following engraftment in the control and CD34KO mouse groups. Mice engrafted with the CD34KO cells (edited with any of the CD34 gRNAs described herein) are expected to have significantly lower levels of hCD34+ cells compared to the mice engrafted with control cells at weeks 8, 12, and 16.

Next, the percentages of particular populations of differentiated cells, such as CD19+ lymphoid cells, hCD14+ monocytes, and hCD11b+ granulocytes/neutrophils in the blood are quantified at weeks 8, 12, and 16 following engraftment in the mice engrafted with CD34KO cells or control cells. The levels of hCD19+ cells, hCD14+ cells, and hCD11b+ cells in the blood are equivalent between the control and CD34KO groups, and the levels of these cells remained equivalent from weeks 8 to 16 post-engraftment. Comparable levels of hCD19+, hCD14+, and hCD11b+ cells in the blood indicate that similar levels of human myeloid and lymphoid cell populations are present in mice that received the CD34KO cells and mice that received the control cells.

Finally, amplicon-seq may be performed on bone marrow samples isolated at week 16 post-engraftment to analyze the on-target CD34 editing in mice that are engrafted with the edited CD34KO cells.

### Evaluating Cell Samples Obtained From the Spleen of Engrafted Animals

At week 16 post-engraftment, the percentages of hCD45+ cells and the percentage of hCD34+ cells are also quantified in the spleen of mice that are engrafted with control cells or CD34KO cells. Comparable levels of hCD45+ cells and reduced levels of hCD34+ cells between the groups of mice (engrafted with control cells or CD34KO cells) may indicate the long-term persistence of CD34KO HSCs in the spleens of NSG mice.

Additionally, at week 16 post engraftment, the percentages of hCD14+ monocytes, hCD11b+ granulocytes/neutrophils, CD19+ lymphoid cells, and hCD3+ T cells in the spleen are quantified. Comparable levels of hCD14+ cells, hCD11b+ cells, hCD19+ cells, and hCD3+ in the spleen between the control and CD34KO groups may indicate that the edited CD34KO cells are capable of multilineage human hematopoietic cell reconstitution in the spleen of the NSG mice.

### Evaluating Blood and Bone Marrow Neutrophils

At week 16 post engraftment, the percentage of hCD11b+ cells are quantified in the blood and the bone marrow of mice engrafted with control cells or CD34KO cells. Comparable levels of CD11b+ neutrophil populations observed in the mice engrafted with control cells and the CD34KO cells in both the blood and the bone marrow of the NSG mice indicates successful engraftment and differentiation.

### Evaluating Blood and Bone Marrow Myeloid and Lymphoid Progenitor Cells

Also, at week 16, the percentage of hCD123+ cells in the blood and the percentage of hCD123+ cells in the bone marrow, and the percentage of hCD10+ cells in the bone marrow are quantified in mice engrafted with control cells or CD34KO cells. Comparable levels of myeloid and lymphoid progenitor cells between the control and CD34KO groups may indicate successful engraftment and development.

### Example 7: Genotyping Analysis of CD34 Edited-HSC Colony Forming Units (CFUs)

This example demonstrates characterization of exemplary CD34-edited HSCs by CFU analysis and INDEL analysis prior to differentiation. The HSCs were edited using exemplary guide RNAs: CD34-2 or CD34-3.

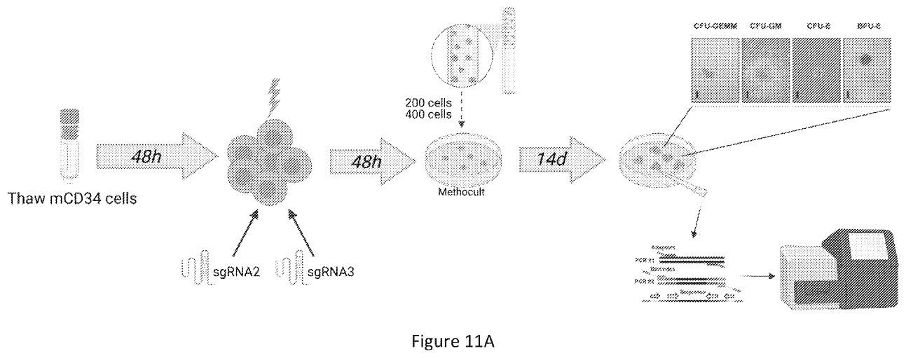

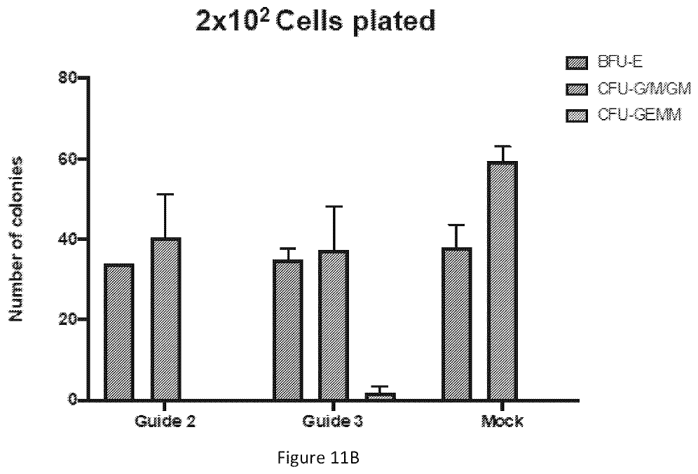

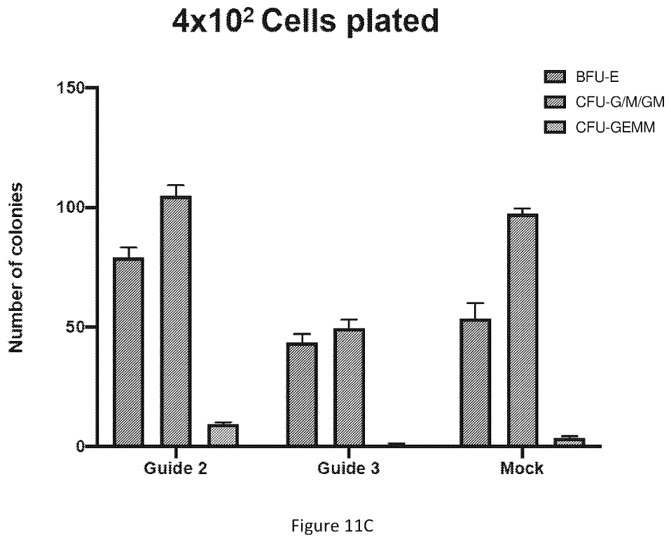

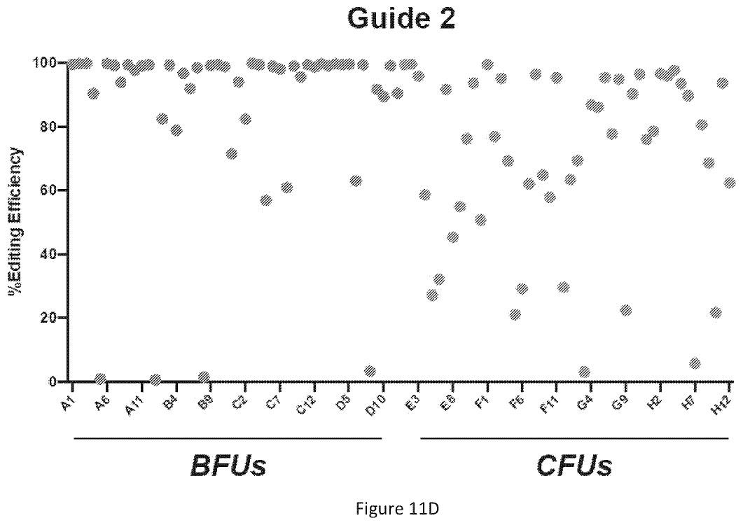

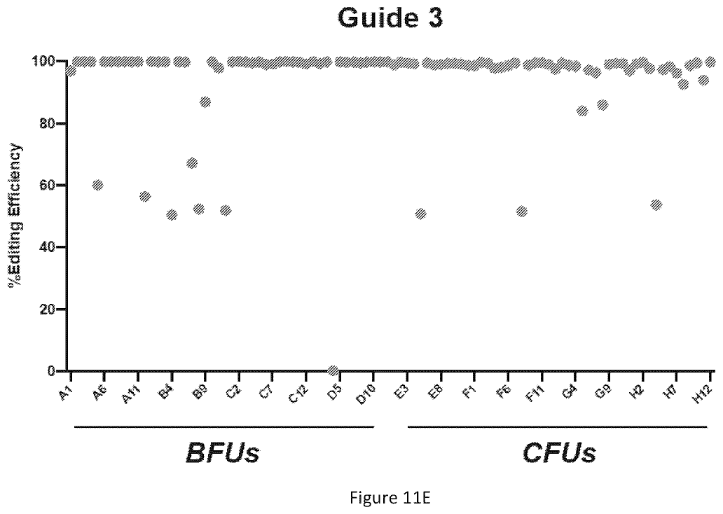

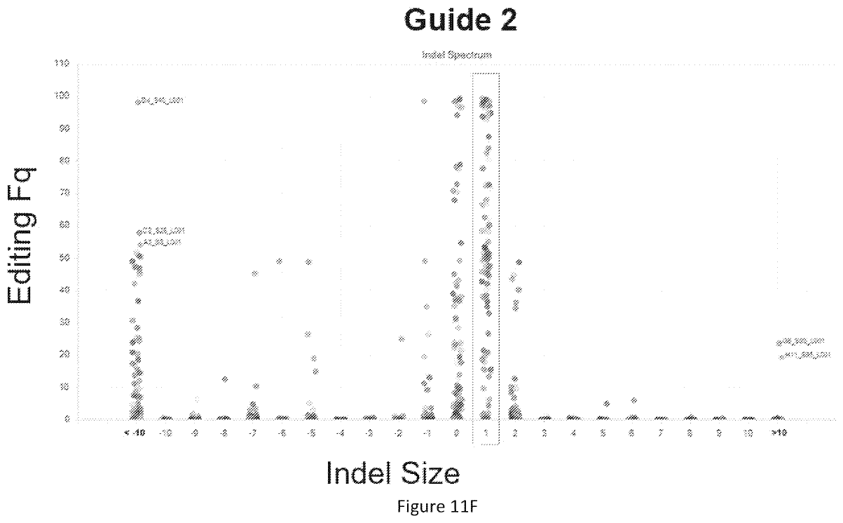

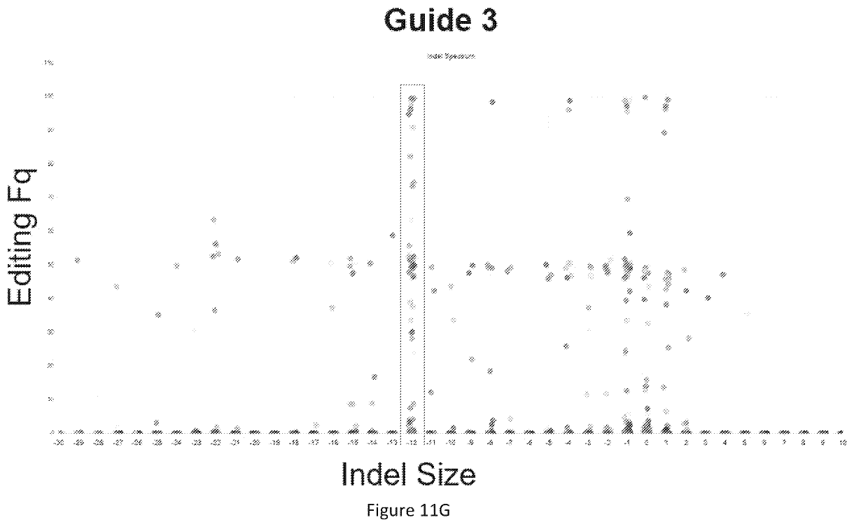

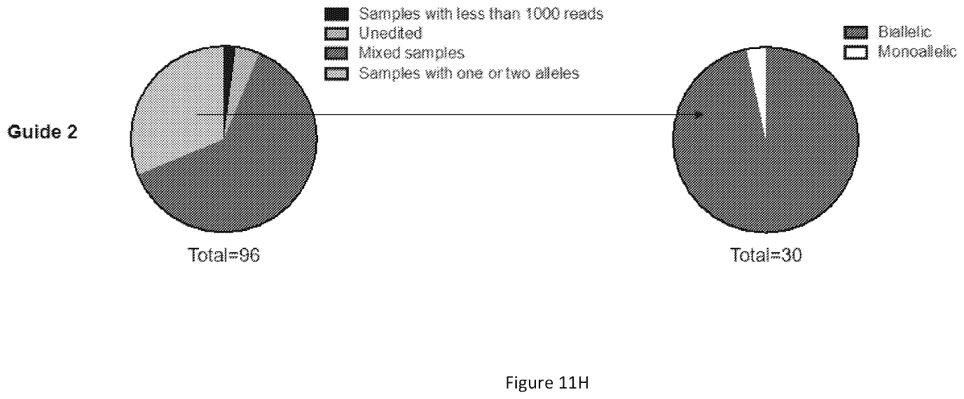

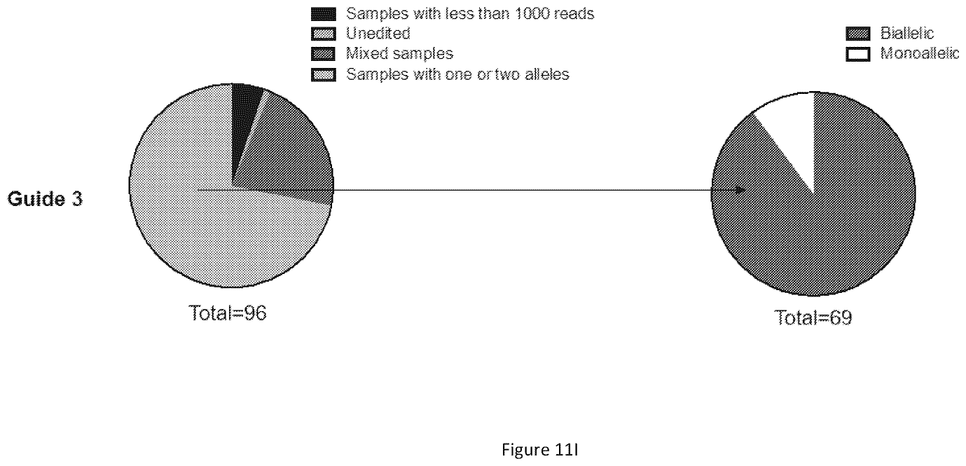

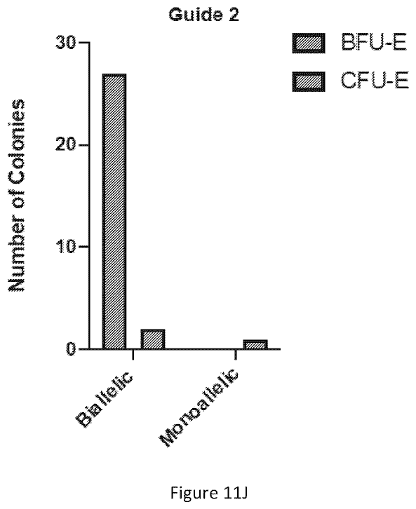

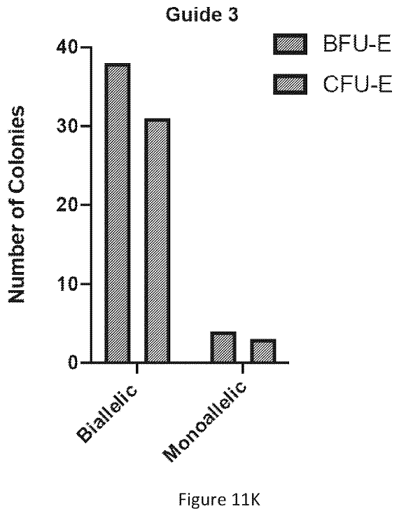

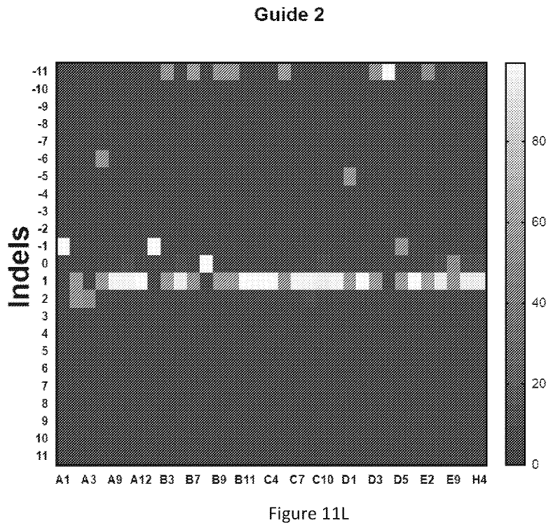

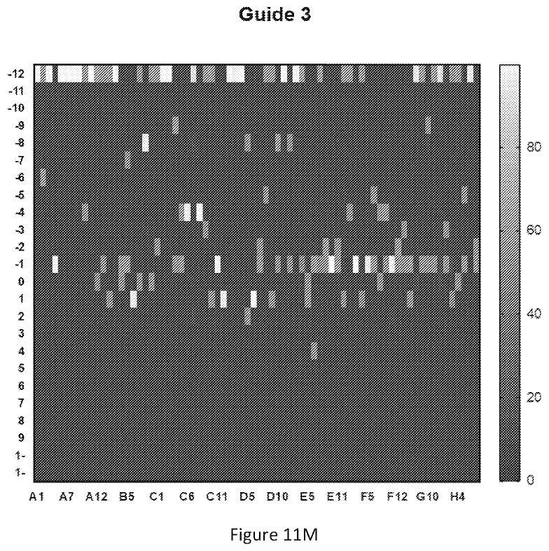

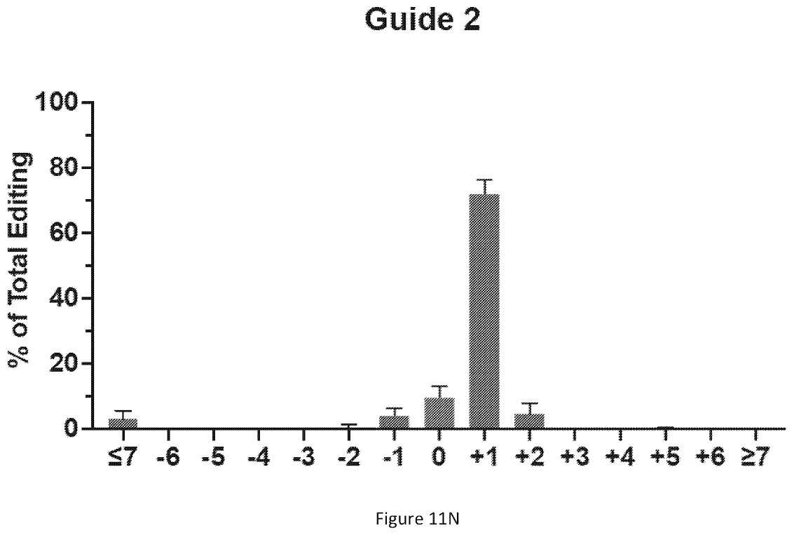

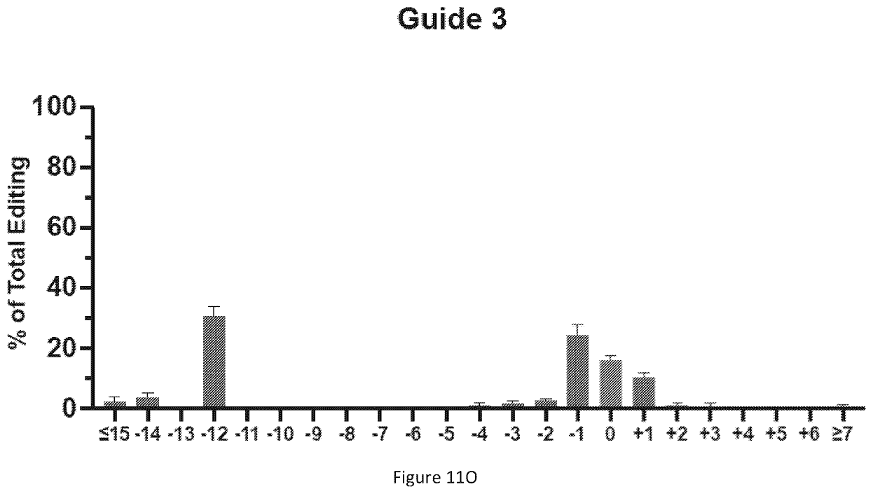

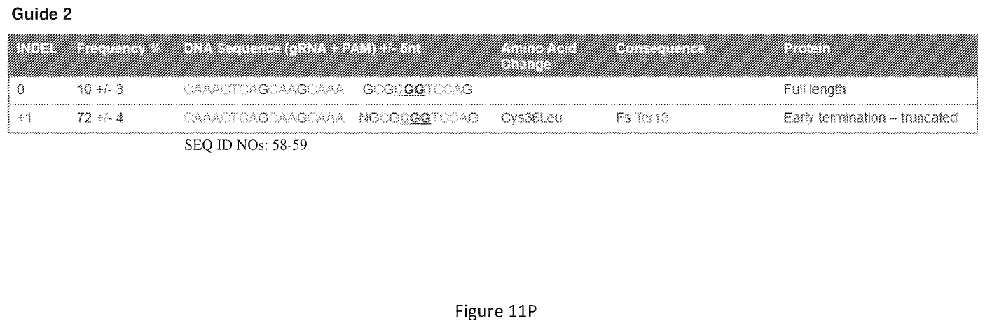

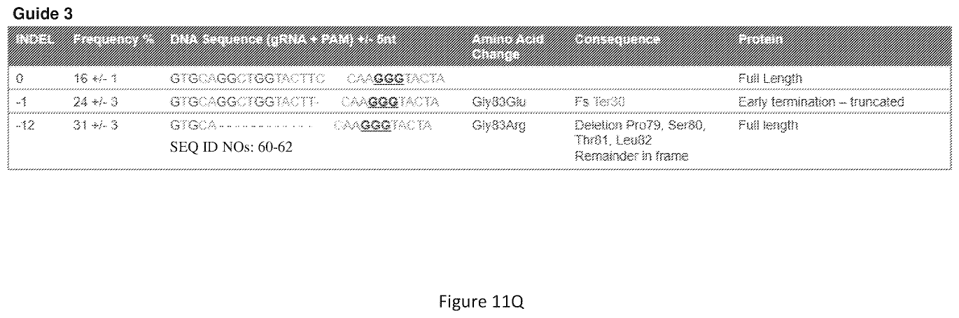

FIG. 11A shows the experimental approach employed for HSC editing in this and later examples. Briefly, 2×10⁶ frozen CD34+ HSCs were thawed and cultured in StemSpan SFEM medium supplemented with StemSpan CC110 cocktail (StemCell Technologies) for approximately 48 h before electroporation with RNP. Approximately 2×10⁵ cells were pelleted and resuspended in electroporation solution and mixed with 3 μg Cas9 and 3 μg gRNA in the form of RNP. CD34+ HSCs were electroporated using standard electroporation procedures. 2×10⁵ cells were included in samples for editing (electroporated with RNP comprising a CD34-2 and Cas9, or with RNP comprising CD34-3 and Cas9), as well as a no electroporation (No EP) control, and a sample electroporated with a control guide (an RNP comprising a control guide RNA that is designed not to target any region in the human genome).

### Evaluation of Edited CD34 HSC CFUs

CFU levels were similar in edited HSCs and HSCs that underwent mock treatment (FIGS. 11B-11C). These results suggest that CD34-editing does not impact cell differentiation. TIDE analyses were used to determine editing efficiencies in individual CD34-edited BFUs and CFUs obtained using CD34-2 (guide 2) and CD34-3 (guide 3). Suitable editing efficiencies were observed in the majority of BFUs and CFUs analyzed (FIGS. 11D and 11E). Analysis of edits generated in CD34-edited HSCs showed that +1 INDELs were the dominant species (72%) in cells edited using CD34-2, resulting in a frameshift leading to early termination of the transcript (FIGS. 11F, 11L, 11N, and 11P. The dominant species of INDEL produced by editing using CD34-2 thus leads to production of a truncated protein. CD34-3 edited cells showed that −12 INDELs were the dominant species (32%), resulting in a deletion of 4 amino acids, followed by a −1 INDEL (24%), resulting in a frameshift leading to early termination of the transcript (FIGS. 11G, 11O, and 11Q).

BFUs and CFUs were further analyzed to determine whether edits were mono- or bi-allelic. Analysis of edits generated in CD34-edited BFUs and CFUs obtained using guides CD34-2 and CD34-3 showed a high percentage of biallelic edits (FIGS. 11H-11K). Biallelic editing was observed for the two guides examined in both CFUs and BFUs (FIGS. 11J and 11K). Indel analysis in individual colonies confirmed the dominant +1 INDEL for guide CD34-2 and the dominant −12 and −1 INDELS for guide CD34-3 (FIGS. 11L-11O). Analysis of the dominant INDEL species for both guides showed that the +1 INDEL for guide CD34-2 results in an early termination and truncation (FIG. 11N), while the dominant −12 INDEL for guide CD34-3 results in a deletion of 4 amino acids in the extracellular domain of CD34, and the next most frequent −1 INDEL for this guide results in an early termination and truncation (FIG. 11Q).

### CD34 Knockout Time Course Studies

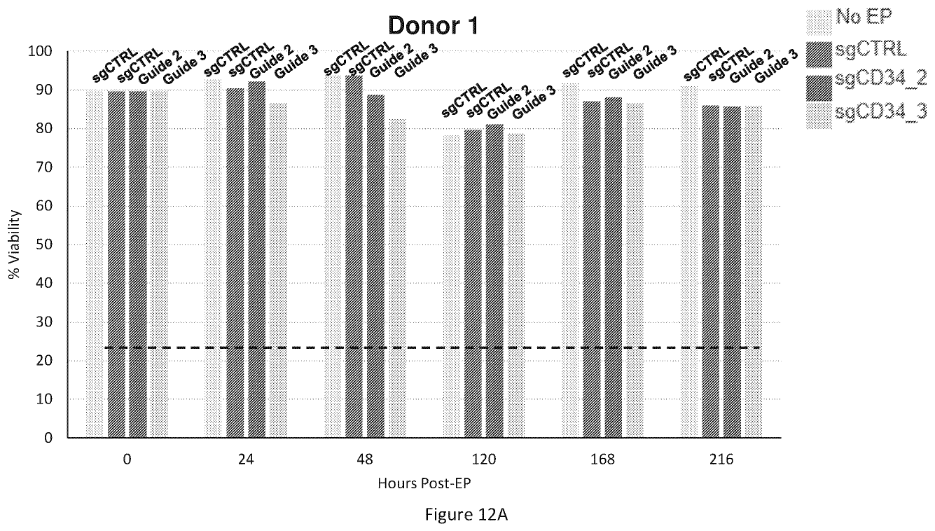

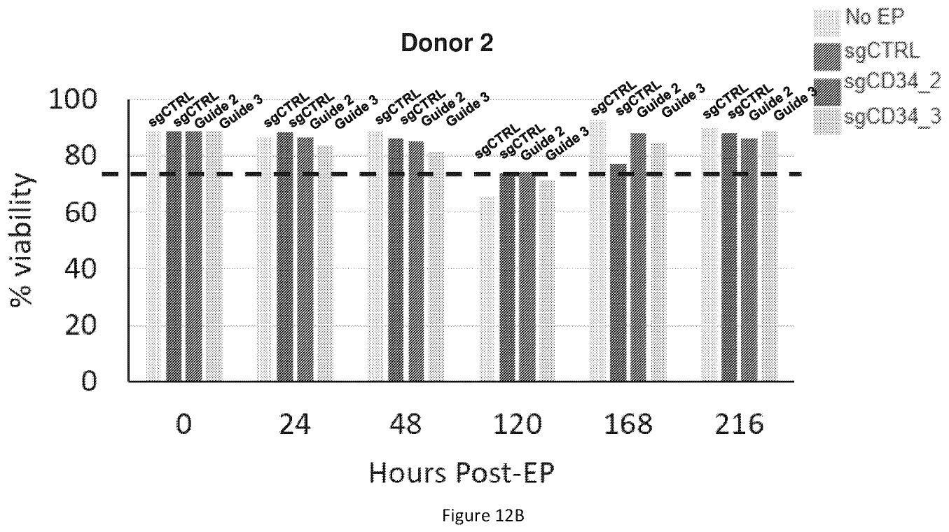

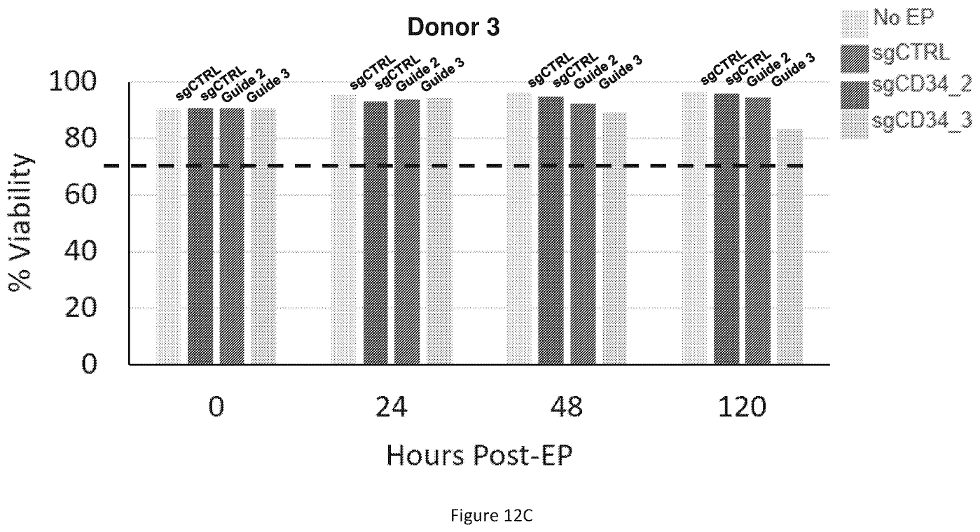

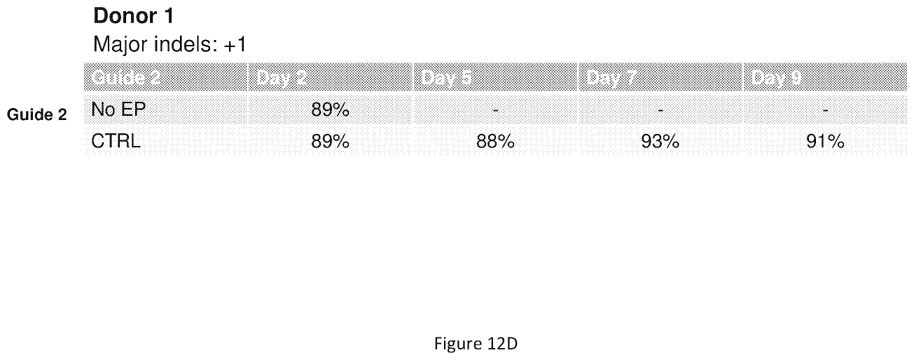

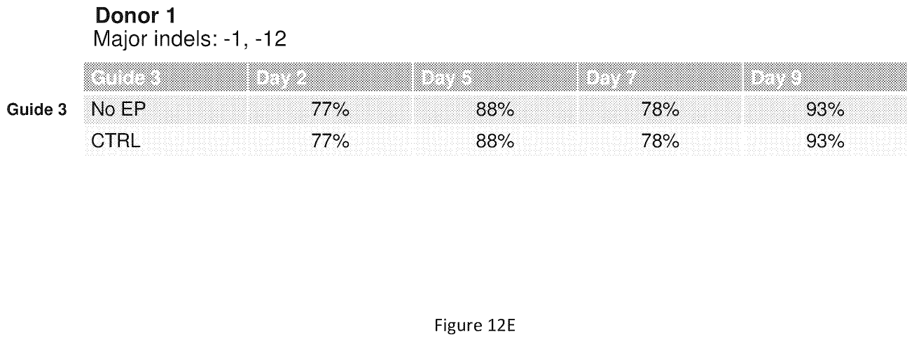

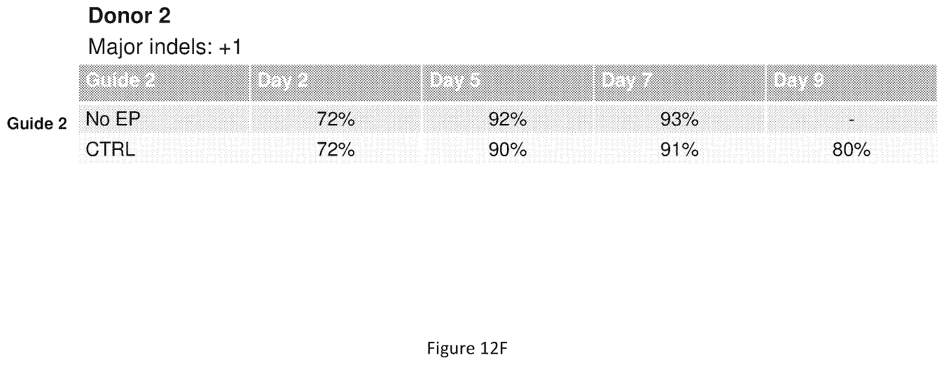

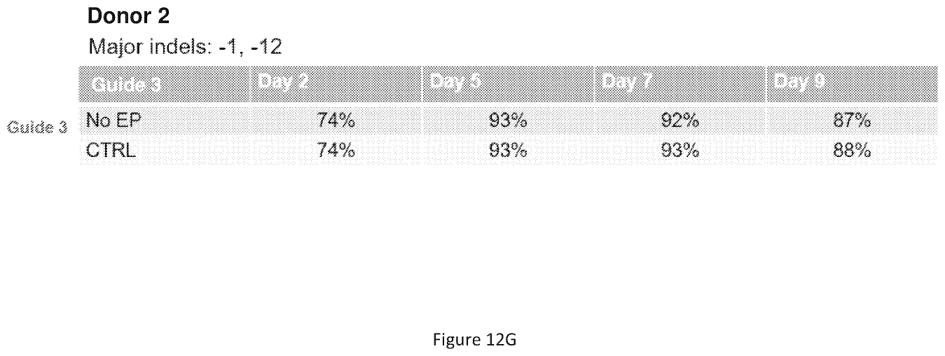

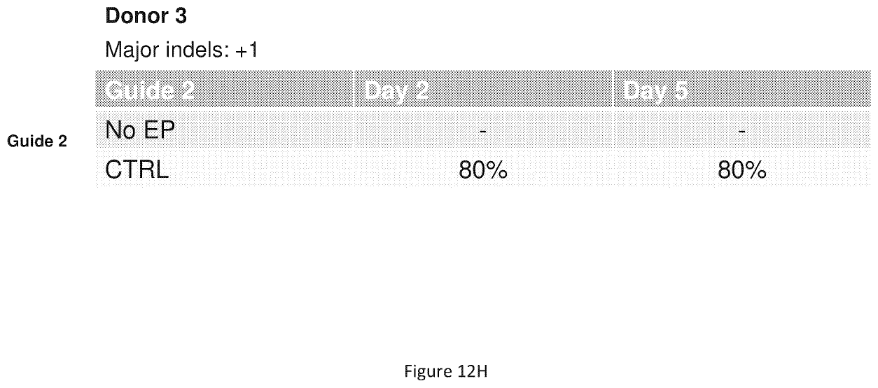

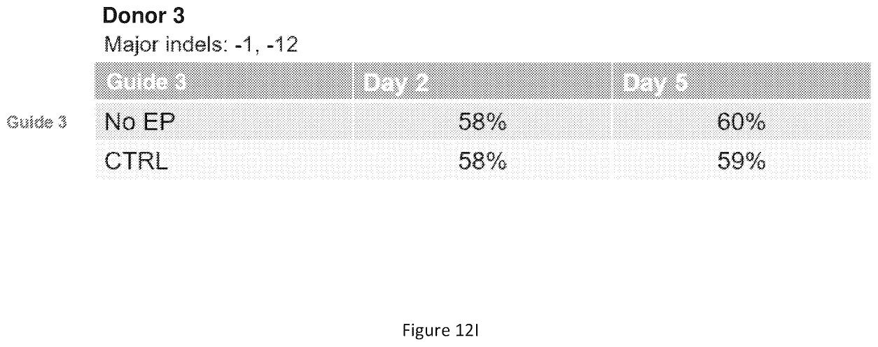

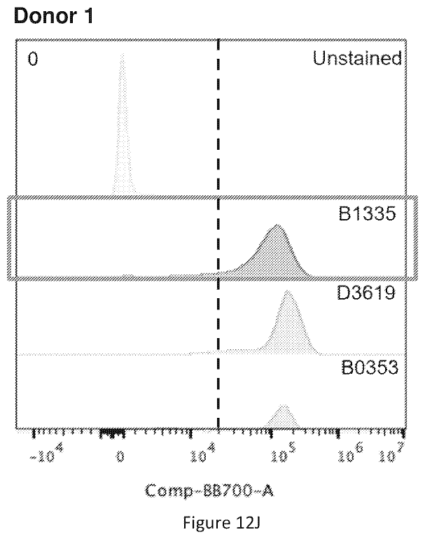

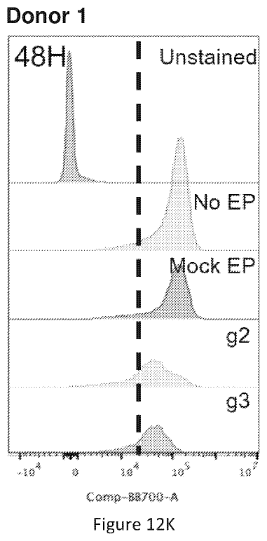

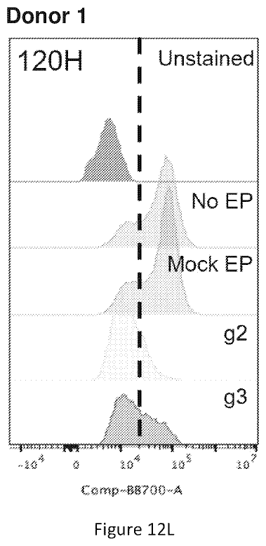

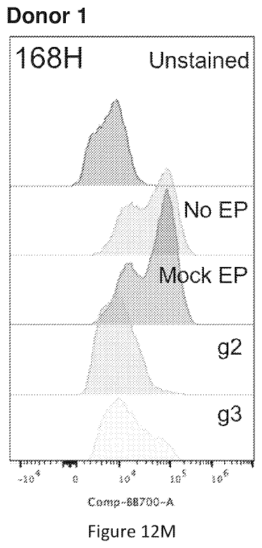

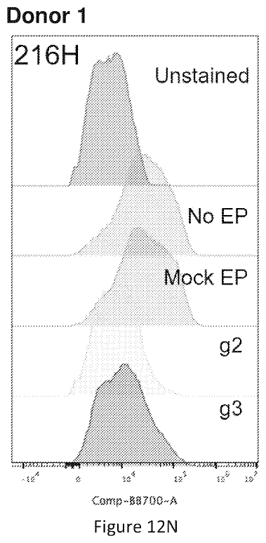

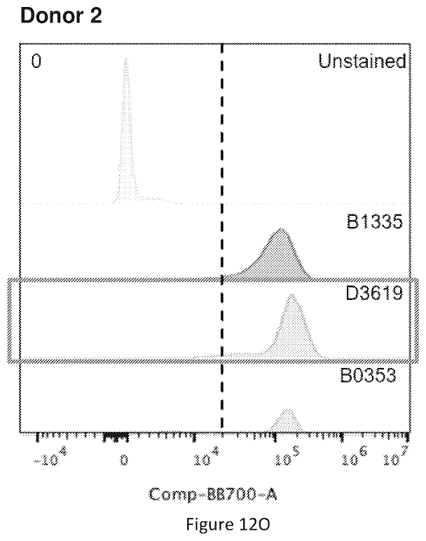

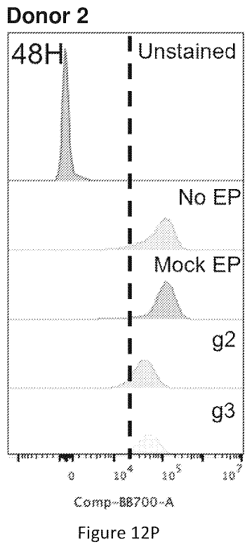

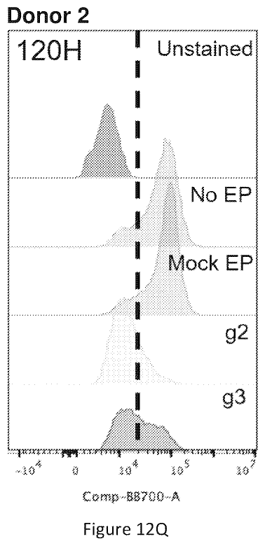

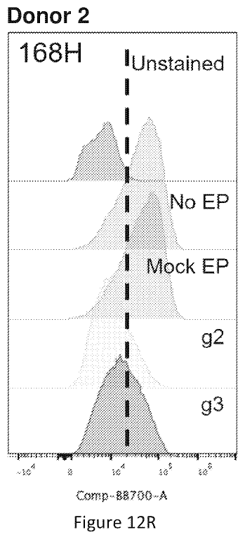

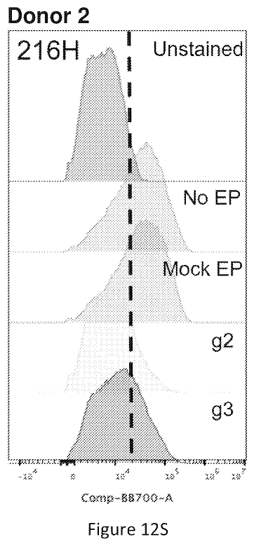

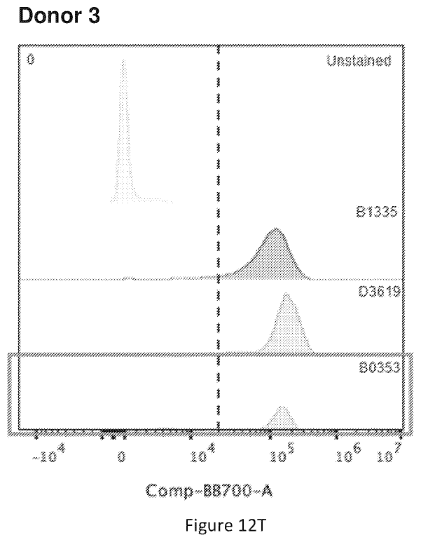

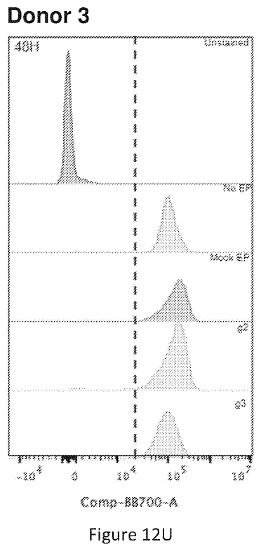

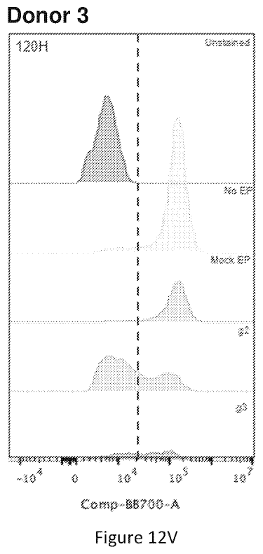

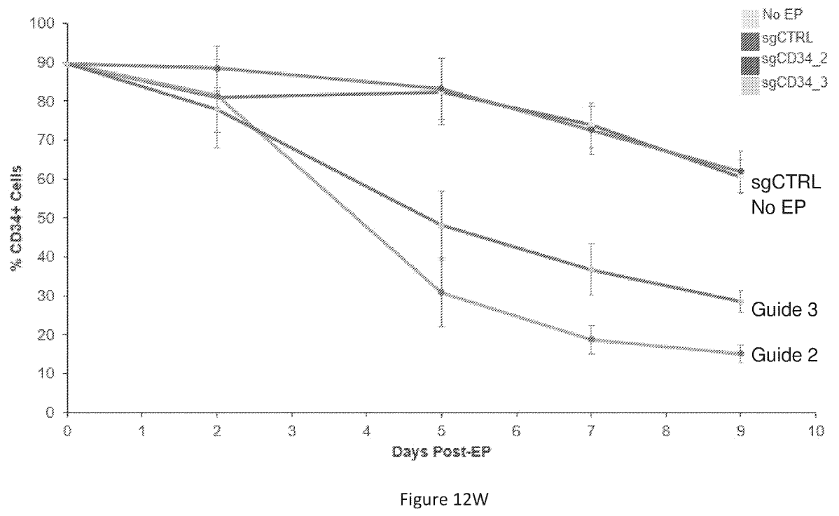

The viability of three different donor HSCs samples (Donor 1, Donor 2, and Donor 3) was assessed after CD34-editing using guide RNAs CD34-2 or CD34-3. Cells were prepared and electroporated as described above. At 24 hours and 48 hours post-ex vivo editing, the average cell counts and percentages of viable, CD34-edited cells (using guides CD34-2 and CD34-3) and control cells (not electroporated (No EP) or electroporated using a control guide not targeting CD34 (sgCTRL), were quantified. CD34-edited cells were viable and remained viable over time following electroporation and gene editing, and no significant differences in viability were observed in CD34-edited cells as compared to the control cells electroporated with a control gRNA, and cells that were not electroporated (FIGS. 12A-12C).

For all genomic analysis, DNA was harvested from cells, amplified with primers flanking the target region, purified and the allele modification frequencies were analyzed using TIDE (Tracking of Indels by Decomposition). ICE analysis of edited cells using both “No EP” and “sgCTRL” data sets as references showed that INDEL generation was detectable at the 48 hour time point and remained present at the 216 hour time point in all three edited donor HSC samples edited with either CD34-2 (FIGS. 12D, 12F, and 12H) or CD34-3 guides (FIGS. 12E, 12G, and 12I). The dominant species of INDELs present in the edited donor HSCs corresponded to those observed in the preceding portion of the Example.

Reductions in surface CD34 expression in edited HSCs were detected by flow cytometry as early as 48 hours post-electroporation in some instances and at 120 hours post-electroporation in all donor samples (FIGS. 12J-12V). These results confirm successful disruption of CD34 in the three donor samples of HSCs using either one of the exemplary guides CD34-2 or CD34-3. The percent of CD34+ cells at different time points was calculated averaging two different donors together for samples edited using guide CD34-2 or CD34-3 as well as for the “No EP” and “sgCTRL” control samples (FIG. 12W). The results and calculated standard deviation show that across donors, editing by the two exemplary guides significantly reduced the frequency of CD34+ cells, producing a population with ˜85% cells showing no detectable CD34 expression at 216 hours post electroporation with CD34-2, and a cell population with more than 70% cells showing no detectable CD34 expression at 216 hours post electroporation with CD34-3.

### Example 8: In Vitro Differentiation (IVD) of CD34 Myeloid Cells

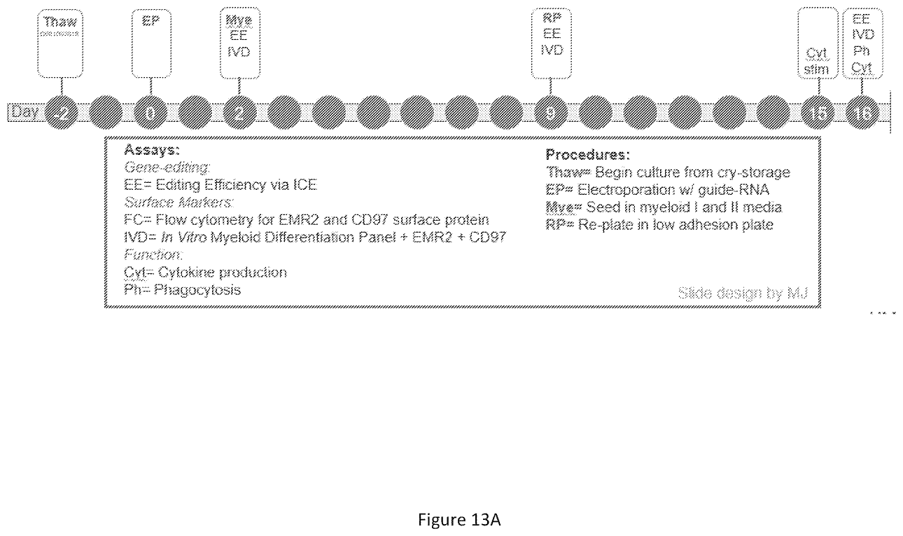

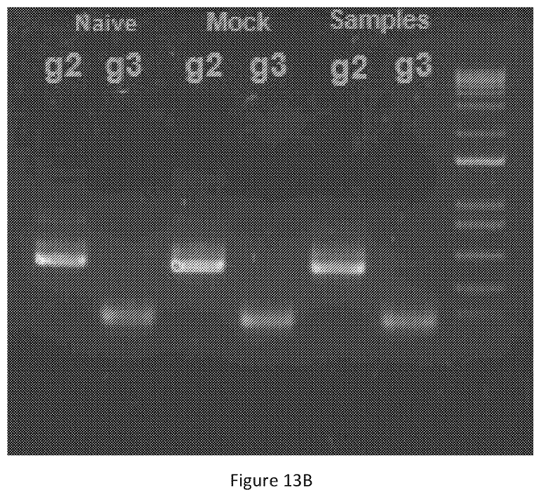

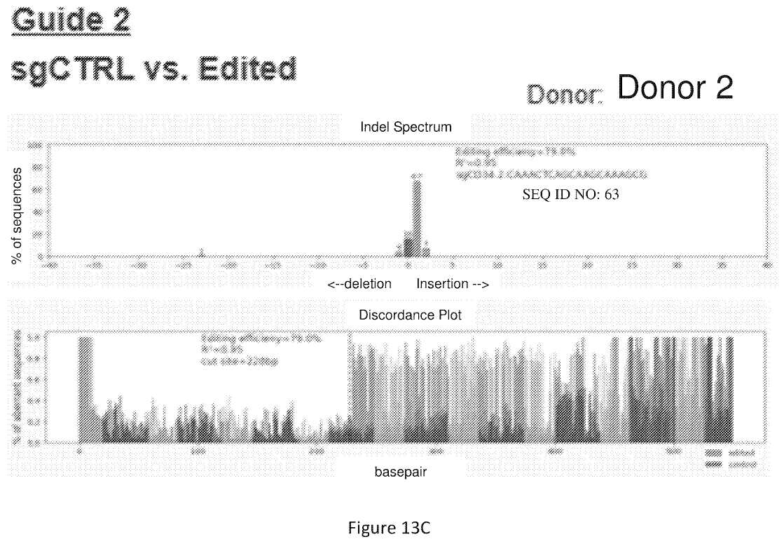

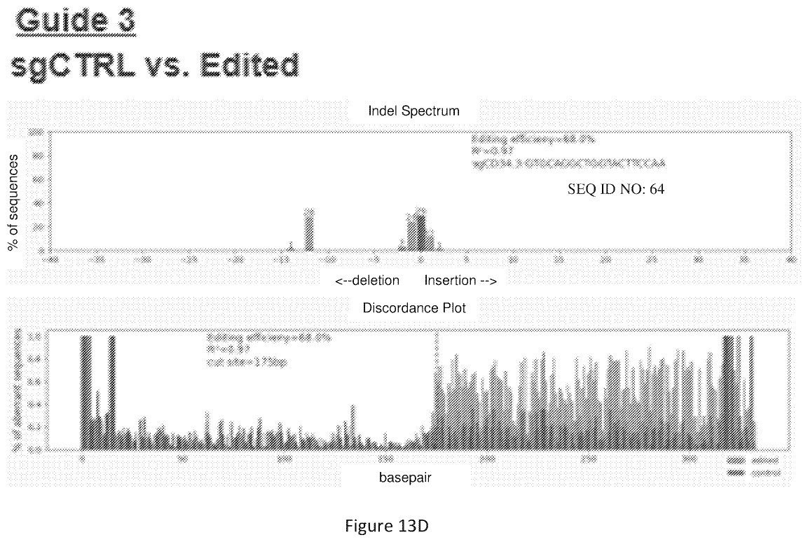

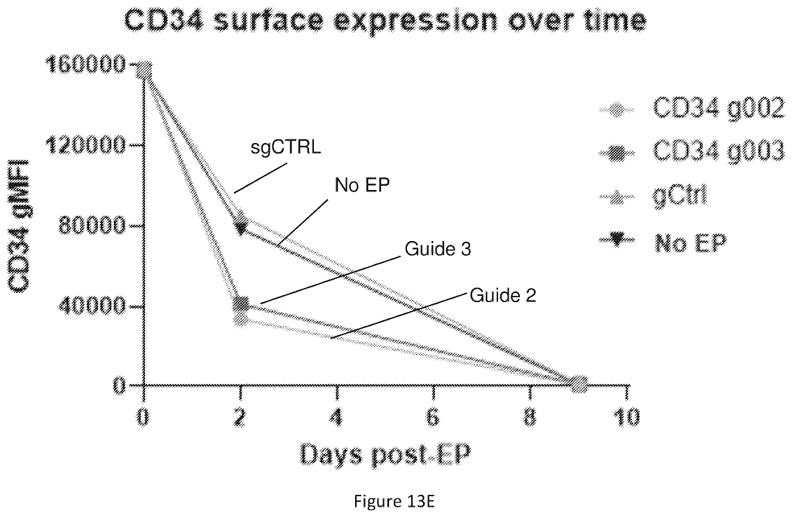

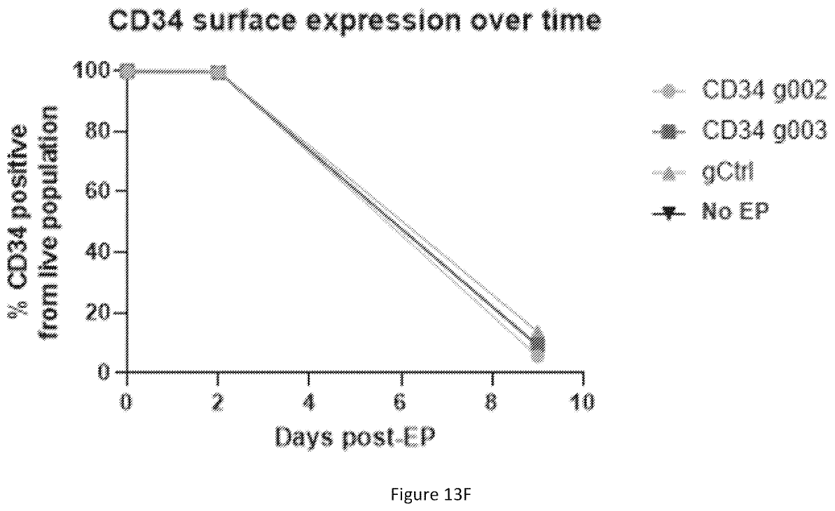

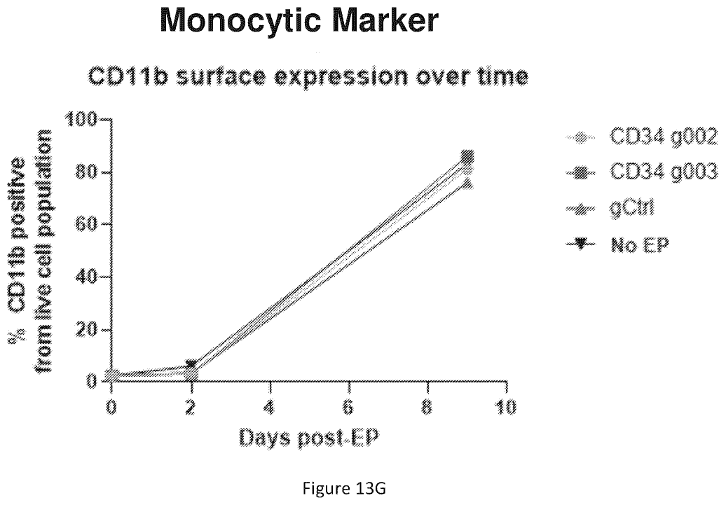

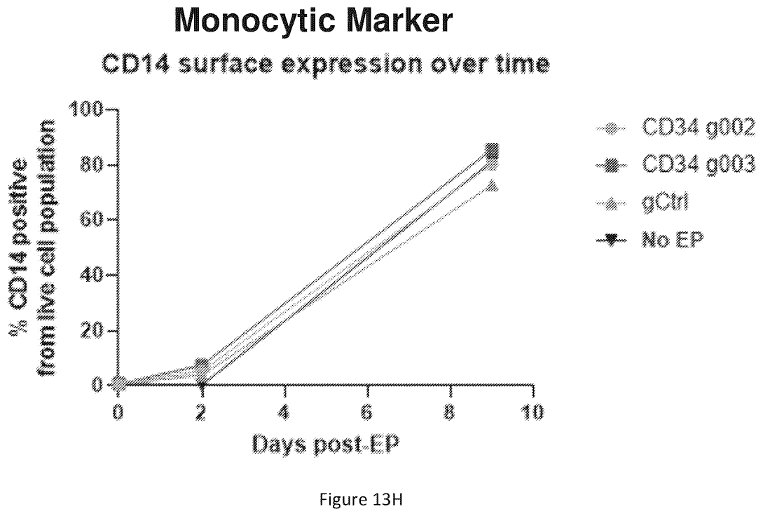

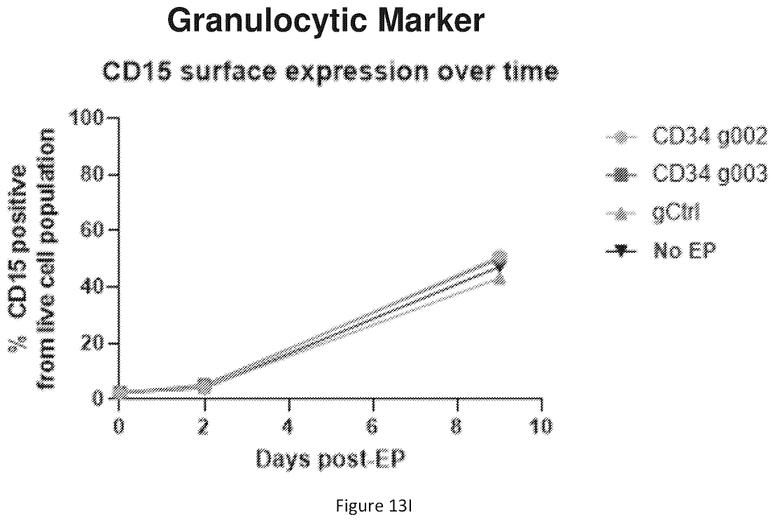

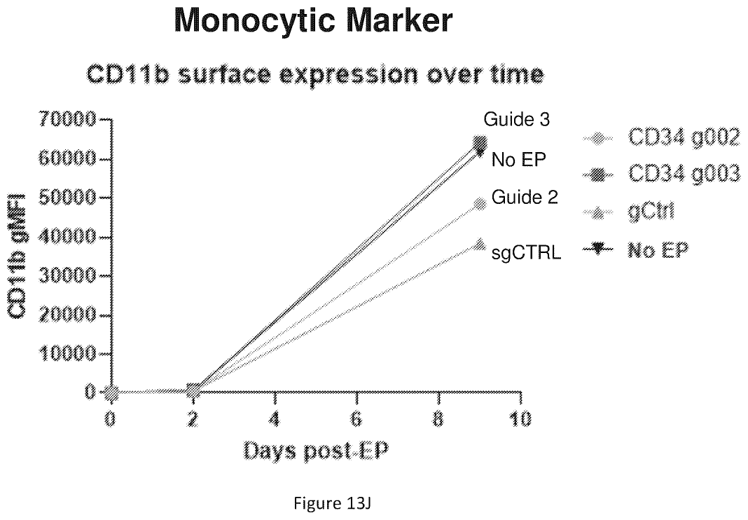

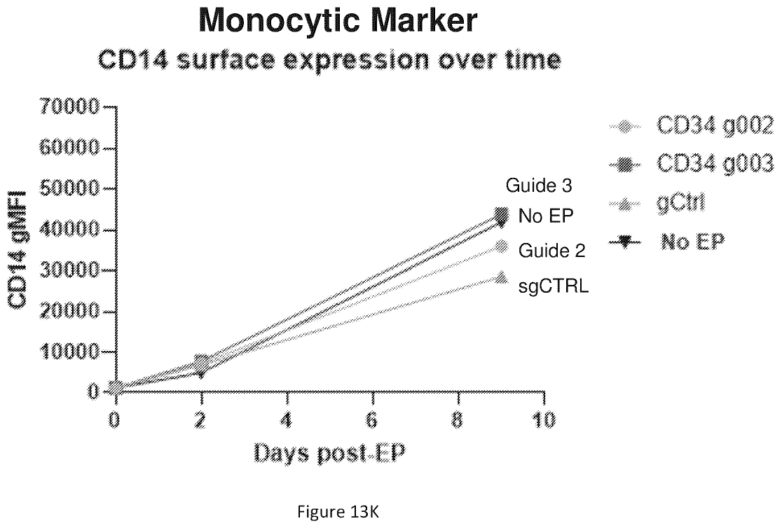

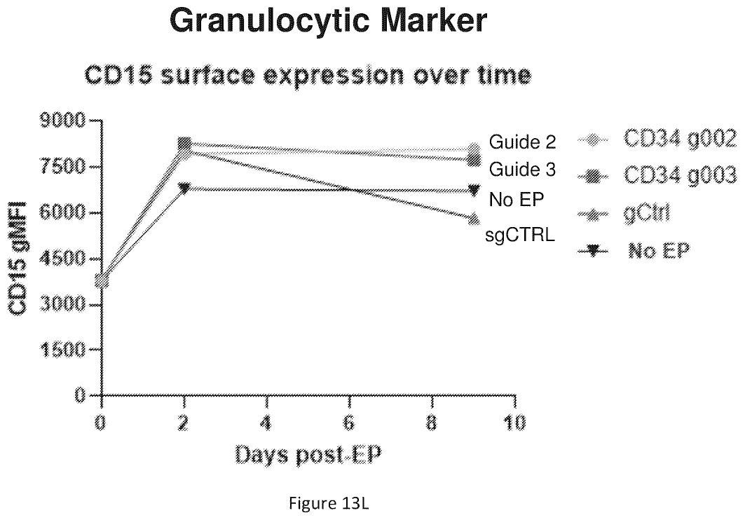

This example demonstrates characterization of the in vitro differentiation capacity of CD34-edited human HSPCs after editing using exemplary guide RNAs provided herein. Cell populations were assessed for myeloid differentiation upon CRISPR/Cas9 editing of CD34 using guide CD34-2 or CD34-3 on various days post differentiation achieved by culturing cells in granulocytic- and monocytic-inducing media conditions. The experimental approach followed is shown FIG. 13A.

### Evaluation of Editing Outcomes in In Vitro Differentiated HSCs

At 48 hours post-electroporation, PCR amplification followed by gel electrophoresis confirmed the presence of edited genomic DNA in cells cultured in IVD media (FIG. 13B). TIDE analysis of DNA harvested from edited HSCs at 48 hours post-electroporation determined the editing efficiency of INDEL generation at the +1 position in CD34-2-treated cells and at the −1 and −12 positions in CD34-3-treated cells confirming successful editing outcomes (FIGS. 13C and 13D). Immunophenotyping of differentiated cells arising from CD34-edited HSCs demonstrated that CD34-editing did not affect the differentiation potential of the CD34-edited cells as compared to No EP and gCTRL control HSCs (FIGS. 13E-13L). No significant differences in expression of differentiation markers was observed between the CD34-edited and the control cell populations. For example, the expression over time of the monocytic surface markers CD11b or CD14 or the CD15 granulocytic markers, assessed as % surface-marker positive cells or by geometric mean fluorescence intensity, were not significantly different in the CD34-edited cell populations as compared to the control cell populations. These results demonstrate that CD34-editing does not disrupt the differentiation capacity of edited HSCs to differentiate into myeloid lineages or induce differentiation bias.

### Example 9: Characterization of CD34-Edited Mice In Vivo

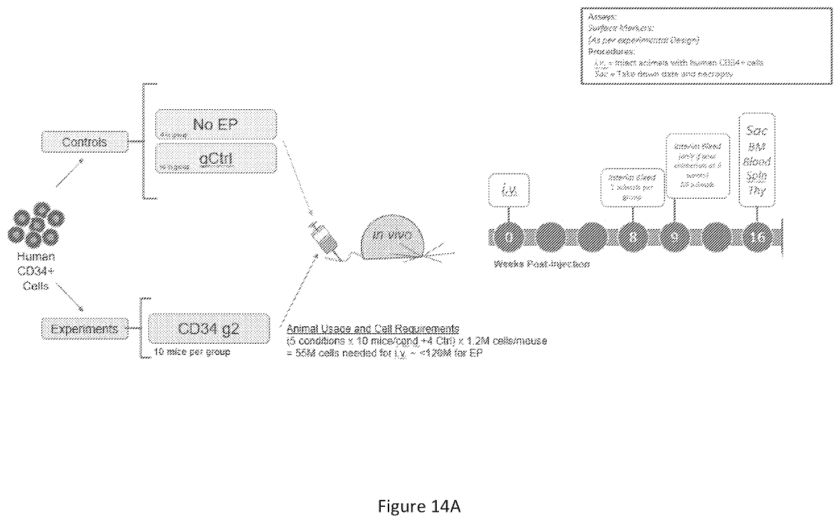

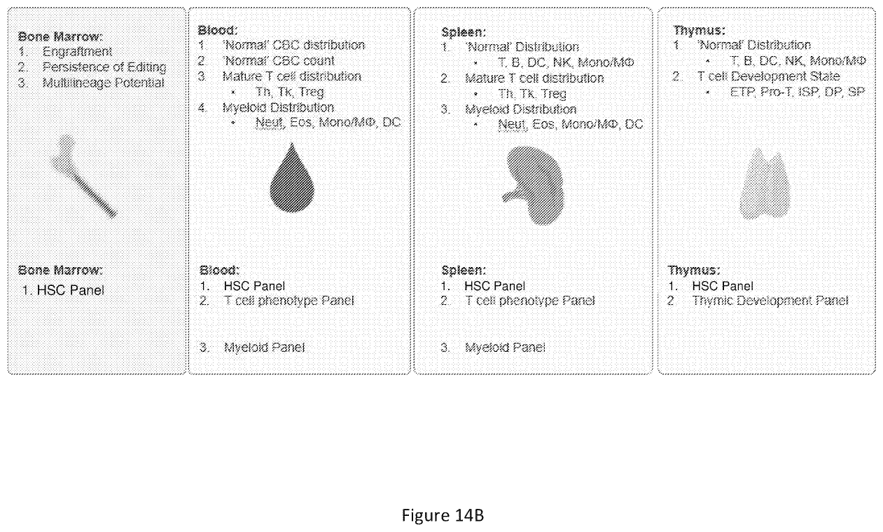

This Example describes in vivo experiments that characterize CD34-edited HSCs in vivo in a mouse model. An exemplary experimental set up is shown in FIG. 14A.

Briefly, human CD34+ HSCs were edited via CRISPR/Cas9 as described herein using the exemplary CD34-targeting guide RNAs (CD34-2 and CD34-3), as well as a non-CD34 targeting control gRNA (gCtrl). At 24 and 48 hours post-ex vivo editing, the percentages of viable, CD34-edited cells and control cells were quantified, and suitable viability (>80%) and editing (>60%) levels were confirmed as described in more detail elsewhere herein. The edited cells were cryopreserved in CS10 media (Stem Cell Technology) at 5×10⁶ cells/mL, in a 1 mL volume of media per vial.

The cryopreserved cells were thawed and counted using a BioRad TC-20 automated cell counter. The number of viable cells were quantified in the thawed vials, and used to prepare the total number of cells for engraftment in the mice. On day 0 of the experiment, female NSG mice (JAX) that are 6 to 8 weeks of age were irradiated using 175 cGy whole body irradiation, and, 4-10 hours after irradiation, the mice were engrafted with the CD34-edited cells generated as described herein or control cells electroporated with gCtrl. Mice were given a single intravenous injection of 1×10⁶ edited cells in a 100 μL volume. Body weight and clinical observations were recorded once weekly for each mouse in the three groups.

At weeks 8, 9, and 12 following engraftment, 50 μL of blood is collected from each mouse by retroorbital bleed for analysis by flow cytometry. At week 16, following engraftment, mice are sacrificed, and blood, spleens, bone marrow, and thymus are collected for analysis by flow cytometry. Bone marrow is isolated from the femur and the tibia. Bone marrow from the femur is also used for on-target editing analysis. Flow cytometry is performed using the Beckman Coulter CytoFLEX LX software. Cells are generally first sorted for viability (e.g., using the 7AAD viability dye (live/dead analysis)), then live cells are gated by expression of human CD45 (hCD45) but not mouse CD45 (mCD45). The cells that are hCD45+ are then further gated for the expression of human lymphoid or myeloid cell surface markers to demonstrate engraftment and repopulation of the blood system by cells derived from the human HSCs. For example, cells expressing human CD45 are analyzed for expression of CD19 (hCD19), in order to detect lymphoid cells, specifically B cells, or for the expression of other lymphoid cell markers, e.g., T-cell markers or NK cell markers. Cells expressing human CD45 (hCD45) are also analyzed for the presence of various cellular markers of the myeloid lineage.

At weeks 8, 12, and 16 following engraftment, the percentage of nucleated blood cells that are hCD45+ is quantified in the groups of mice (n=10 mice/group) that received control cells edited with the control gRNA (gCtrl), or the CD34-edited cells. This value is calculated by dividing the hCD45+ absolute cell count by the mouse CD45+ (mCD45) absolute cell count.

The percentage of hCD34+ cells in the blood is also quantified at week 8 following engraftment in the control and CD34-edited mouse groups as an assessment for human and mouse cell chimerism. Mice engrafted with the CD34-edited cells (e.g., edited with CD34-2 as described herein) have similar levels of hCD34+ cells compared to the mice engrafted with control cells (not CD34-edited, e.g., no electroporation or edited using a control guide not targeting CD34) at weeks 8, 12, and 16.

Next, the percentages of particular populations of differentiated cells is analyzed. For example, the percentages of CD19+ lymphoid cells, hCD14+ monocytes, and hCD11b+granulocytes/neutrophils in the blood are quantified at weeks 8, 12, and 16 following engraftment in the mice engrafted with CD34-edited cells or control cells. The levels of hCD19+ cells, hCD14+ cells, and hCD11b+ cells in the blood are equivalent between the control and CD34-edited groups, and the levels of these cells remain equivalent from weeks 8 to 16 post-engraftment. Comparable levels of hCD19+, hCD14+, and hCD11b+ cells in the blood are observed, indicating successful engraftment and differentiation of the CD34-edited HSCs in the irradiated NSG mice, and demonstrating that similar levels of human myeloid and lymphoid cell populations are present in mice that receive the CD34-edited cells and mice that receive the control cells.

Presence and frequency of CD34-editing is confirmed in engrafted cells in the NSG animals that received the CD34-edited cell populations. Amplicon-seq is performed on bone marrow samples isolated at week 16 post-engraftment to analyze the CD34 editing in mice engrafted with the edited CD34-edited cells. The presence of CD34-edited cells in the bone marrow is confirmed, demonstrating the ability of the CD34-edited cells to successfully engraft. The presence of CD34-edited HSCs is confirmed by immunophenotyping bone marrow-derived cells or circulating PBMC cells and identifying CD34-edited cells that are CD34−, CD45RA−, CD49c+, CD90+, CD201+, and lin−.

At week 16 post engraftment, the percentage of hCD11b+ cells are quantified in the blood and the bone marrow of mice engrafted with control cells or CD34-edited cells. Comparable levels of CD11b+ neutrophil populations observed in the mice engrafted with control cells and the CD34-edited cells in both the blood and the bone marrow of the NSG mice indicates successful engraftment and differentiation.

At week 16 post-engraftment, the percentages of hCD45+ cells and the percentage of hCD34+ cells are also quantified in the spleen of mice that are engrafted with control cells or CD34-edited cells. Comparable levels of hCD45+ cells and reduced levels of hCD34+ cells between the groups of mice (engrafted with control cells or CD34-edited cells) may indicate the long-term persistence of CD34-edited HSCs in the spleens of NSG mice. Additionally, at week 16 post engraftment, the percentages of hCD14+ monocytes, hCD11b+ granulocytes/neutrophils, CD19+ lymphoid cells, and hCD3+ T cells in the spleen are quantified. Comparable levels of hCD14+ cells, hCD11b+ cells, hCD19+ cells, and hCD3+ in the spleen between the control and CD34-edited groups may indicate that the CD34-edited cells are capable of multilineage human hematopoietic cell reconstitution in the spleen of the NSG mice.

At week 16 post-engraftment, the percentages of hCD45+ cells and the percentage of hCD34+ cells are also quantified in the thymus of mice that are engrafted with control cells or CD34-edited cells. Additionally, at week 16 post engraftment, the percentages of CD3+ cells (as a percent of hCD45+ cells) in the thymus are quantified. Comparable levels of hCD3+ in the thymus between the control and CD34-edited groups may indicate that the CD34-edited cells are capable of multilineage human hematopoietic cell reconstitution in the thymus of the NSG mice.

### Example 10: Ablation of CD34+ HSCs in a Subject Having a Hematopoietic Disorder

A subject having a hematopoietic disorder, e.g., a blood malignancy, in which malignant cells (for example, cancer stem cells) express CD34, is treated with an allogeneic hematopoietic cell transplant (HCT) comprising CD34-edited HSCs and with an anti-CD34 immunotherapeutic, for example, with a CD34 antibody conjugated to a calicheamicin moiety.

For the HCT, a population of cells comprising CD34-edited hematopoietic stem cells is obtained according to the methods described herein, for example, by obtaining HSCs from a healthy donor is HLA matched at 8/8 loci (HLA-A, -B, -C, DRB1) to the subject, and editing the HSCs using guide CD34-2 or CD34-3.

HSCs are obtained from the donor after G-CSF/plerixafor mobilization in up to two apheresis procedures. A minimum of 10×10⁶ viable cells/kg (where kg refers to recipient subject weight) are obtained from the donor by apheresis for processing, editing, and subsequent administration to the recipient subject. From this apheresis product, at least 3.0×10⁶ viable cells/kg (recipient weight) undergo minimal manipulation and are cryopreserved to serve as a back-up stem cell source, e.g., for use as a rescue dose. The remainder of the apheresis product is used for processing and preparation of the CD34-edited HSC population for HCT.

The CD34-edited HSC population for HCT is prepared by enriching the apheresis product for CD34+ cells, followed by electroporation and editing with a CD34 gRNA/Cas9 complex, as described above, using CD34 guide CD34-2 or CD34-3 as described herein.

The CD34-edited cells are subsequently placed in culture for about 48 hours. Upon harvest, after the culture duration is finished, cells are washed, resuspended in the final formulation, and cryopreserved. Cell viability and editing efficiency are confirmed using a representative sample, and CD34-edited HSC populations confirmed to comprise at least 70% viable cells and at least 45% CD34 editing efficiency are used for HCT. A CD34-edited cell population for administration to a subject comprises a CD34-edited HSC population satisfying these viability and editing efficiency criteria and comprising at least 3×10⁶ cells/kg body weight of the recipient subject, and preferably comprises at least 4×10⁶ cells/kg, 5×10⁶ cells/kg, 6×10⁶ cells/kg, or 7×10⁶ cells/kg of the recipient subject.

The subject is conditioned using routine clinical procedures to ablate the subject's hematopoietic stem cells, including any malignant cells expressing CD34. After completion of the conditioning regimen, the subject receives an HCT comprising the thawed CD34-edited HSCs via an intravenous (IV) infusion. The day of the HCT is day 0 of the treatment regimen.

The subject is assessed for CD34-edited HSC engraftment at day 28 by measuring the absolute peripheral neutrophil count (ANC) for CD34-edited (CD34−, e.g., truncated CD34 or epitope-edited CD34) neutrophils in the subject. The subject is deemed to exhibit neutrophil recovery (also referred to as successful CD34-edited neutrophil engraftment) if the subject exhibits an absolute peripheral CD34-edited neutrophil count of ≥1000/dL CD33− ANC at 28 days after HCT.

If the subject exhibits neutrophil recovery at day 28, a bone marrow biopsy is obtained from the subject on day 60 in order to assess disease status and hematopoietic recovery. In addition, percent donor chimerism and CD34-edited myeloid hematopoiesis are determined from the peripheral blood at this time. If the subject exhibits successful CD34-edited HSC engraftment and CD34-edited hematopoiesis at day 60, the subject is subsequently administered a CD34-antibody-drug-conjugate. Administration of the CD34-targeted immunotherapeutic, e.g., an anti-CD34 antibody-drug-conjugate, is preferably initiated within 30 days of the bone marrow biopsy at day 60, i.e., is preferably initiated by day 90. However, initiation of the CD34-targeted immunotherapeutic may be delayed up to day 120 if a subject's clinical status, e.g., in view of comorbidities, including, for example, HCT-related comorbidities, necessitate such a delay, or in order to allow attainment of ≥1000/dL CD33-ANC in a subject. If the CD34-targeted immunotherapeutic is initiated more than 30 days after the day 60 bone marrow biopsy, a repeat bone marrow biopsy is completed prior to starting the CD34-targeted immunotherapeutic.

The CD34-targeted immunotherapeutic is administered at its recommended dose to ablate all CD34-expressing hematopoietic cells in the subject. However, some subjects may be administered a lower dose, e.g., in the event of treatment-related adverse effects, e.g., dose-limited toxicities (DLT), or in view of the health status, comorbidities, or the medical history of the individual subject.

Alternatively, the subject is not subjected to a conventional conditioning regimen in order to ablate the subject's own CD34-expressing blood cells prior to HCT, but the CD34-expressing cells of the subject are targeted by administration of a CD34-targeted immunotherapeutic, such as a CD34-targeting antibody-drug conjugate or a CD34-targeting CAR-T cell population. In such a scenario, the subject may be administered a cycle of the immunotherapeutic before the HCT of CD34-edited HSCs, and a cycle of the immunotherapeutic after the HCT. Alternatively, the subject may be administered the immunotherapeutic subsequent to the HCT, in one or more cycles, thus completely foregoing a pre-HCT conditioning regimen.

After administration of the immunotherapeutic, the subject is assessed for the presence of malignant cells. A significant reduction in the number of malignant cells, in particular of CD34-expressing malignant cells, or a complete ablation of CD34-expressing malignant cells is observed in the subject. In addition, long-term engraftment of the CD34-edited HSCs and reconstitution of all cell lineages from the CD34-edited HSCs are observed in the subject.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any clement or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth explicitly herein.

What is claimed is:

1. A genetically engineered hematopoietic cell, or descendant thereof, comprising a modified gene encoding CD34.

2. The hematopoietic cell, or descendant thereof, of claim 1, wherein the modified gene encoding CD34 comprises an INDEL mutation.

3. The hematopoietic cell, or descendant thereof, of claim 1 or 2, wherein the modified gene encoding CD34 is modified such that an exon is skipped.

4. The hematopoietic cell, or descendant thereof, of any one of claims 1-3, wherein exon 1, exon 2, or exon 3 of the modified gene encoding CD34 is genetically engineered.

5. The hematopoietic cell, or descendant thereof, of any one of claims 1-4, wherein the modified gene encoding CD34 comprises an insertion or deletion immediately proximal to a site cut by an RNA-guided nuclease when bound to a gRNA comprising a targeting domain as provided by any one of SEQ ID NOs: 11-15.

6. The hematopoietic cell, or descendant thereof, of any one of claims 1-5, wherein the modified gene encoding CD34 comprises an insertion or deletion generated by a non-homologous end joining (NHEJ) event.

7. The hematopoietic cell, or descendant thereof, of any one of claims 1-5, wherein the modified gene encoding CD34 comprises an insertion or deletion generated by a homology-directed repair (HDR) event.

8. The hematopoietic cell, or descendant thereof, of any one of claims 1-7, wherein expression of CD34 is reduced or eliminated relative to a wild-type counterpart cell that does not harbor the modified gene encoding CD34.

9. The hematopoietic cell, or descendant thereof, of claim 8, expression of CD34 is less than 25%, less than 20% less than 10% less than 5% less than 2% less than 1%, less than 0.1%, less than 0.01%, or less than 0.001% as compared to the expression level of CD34 in the wild-type counterpart cell that does not harbor a modified gene encoding CD34.

10. The hematopoietic cell, or descendant thereof, of any one of claims 1-9, wherein the hematopoietic cell, or descendant thereof, has reduced or no binding to an agent comprising an anti-CD34 binding domain.

11. The hematopoietic cell, or descendant thereof, of any one of claims 1-10, wherein the hematopoietic cell, or descendant thereof retains the capacity to differentiate normally compared to a population of hematopoietic cells that are not genetically engineered.

12. The hematopoietic cell, or descendant thereof, of any one of claims 1-11, wherein the modified gene encoding CD34 results in a loss of function of CD34 in the genetically engineered hematopoietic cell, or descendant thereof.

13. The hematopoietic cell, or descendant thereof, of any one of claims 1-12, further comprising a modified gene encoding a lineage-specific cell-surface antigen.

14. The hematopoietic cell, or descendant thereof, of claim 13, wherein the lineage-specific cell-surface antigen is CD33, CD123, CLL-1, CD19, CD30, CD5, CD6, CD7, CD38, or BCMA.

15. The hematopoietic cell, or descendant thereof, of claim 13 or 14, wherein the hematopoietic cell, or descendant thereof has reduced or no binding to an agent comprising a binding domain that targets the lineage-specific cell-surface antigen.

16. A method comprising administering to a subject in need thereof the genetically engineered hematopoietic cell, or descendant thereof, of any one of claims 1-15.

17. The method of claim 16, further comprising administering to the subject a therapeutically effective amount of at least one agent that targets CD34, wherein the agent comprises an antigen binding fragment that binds CD34.

18. The method of claim 17, wherein the agent is an antibody-drug conjugate or an immune effector cell expressing a chimeric antigen receptor (CAR).

19. The method of any one of claims 16-18, wherein the subject has a disease associated with cells expressing CD34.

20. The method of any one of claims 16-19, wherein the subject has a malignancy associated with or characterized by the expression of CD34 on malignant cells, optionally wherein the malignant cells are cancer stem cells.

21. The method of any one of claims 16-20, wherein the subject has a hematopoietic malignancy.

22. The method of any one of claims 16-20, wherein the subject has an autoimmune disease.

23. A gRNA comprising a targeting domain, wherein the targeting domain comprises a sequence selected from the group consisting of SEQ ID NOs: 11-15.

24. The gRNA of claim 23, wherein the gRNA comprises a first complementarity domain, a linking domain, a second complementarity domain which is complementary to the first complementarity domain, and a proximal domain.

25. The gRNA of claim 23 or 24, wherein the gRNA is a single guide RNA (sgRNA).

26. The gRNA of any one of claims 23-25, wherein the gRNA comprises one or more nucleotide residues that are chemically modified.

27. The gRNA of any one of claims 23-26, wherein the gRNA comprises one or more nucleotide residues that comprise a 2′O-methyl moiety.

28. The gRNA of any one of claims 23-27, wherein the gRNA comprises one or more nucleotide residues that comprise a phosphorothioate.

29. The gRNA of any one of claims 23-28, wherein the gRNA comprises one or more nucleotide residues that comprise a thioPACE moiety.

30. A method of producing a genetically engineered cell, comprising:
a. providing a cell, and
b. contacting the cell with (i) a gRNA of any of claims 23-29; and (ii) an RNA-guided nuclease that binds the gRNA, thus forming a ribonucleoprotein (RNP) complex under conditions suitable for the gRNA of (i) to form and/or maintain an RNP complex with the RNA-guided nuclease of (ii) and for the RNP complex to bind a target domain in the genome of the cell.

31. The method of claim 30, wherein the RNA-guided nuclease is a CRISPR/Cas nuclease.

32. The method of claim 31, wherein the CRISPR/Cas nuclease is a Cas9 nuclease.

33. The method of claim 31, wherein the CRISPR/Cas nuclease is an spCas nuclease.

34. The method of claim 31, wherein the CRISPR/Cas nuclease in an saCas nuclease.

35. The method of claim 31, wherein the CRISPR/Cas nuclease is a Cpf1 nuclease.

36. The method of any one of claims 30-35, wherein the contacting comprises introducing (i) and (ii) into the cell in the form of a pre-formed ribonucleoprotein (RNP) complex.

37. The method of any one of claims 30-36, wherein the contacting comprises introducing (i) and/or (ii) into the cell in the form of a nucleic acid encoding the gRNA of (i) and/or the RNA-guided nuclease of (ii).

38. The method of claim 37, wherein the nucleic acid encoding the gRNA of (i) and/or the RNA-guided nuclease of (ii) is an RNA, preferably an mRNA or an mRNA analog.

39. The method of any one of claims 30-38, wherein the ribonucleoprotein complex is introduced into the cell via electroporation.

40. The method of any one of claims 30-39, wherein the cell is a hematopoietic cell.

41. The method of any one of claims 30-40, wherein the cell is a hematopoietic stem cell.

42. The method of any one of claims 30-41, wherein the cell is a hematopoietic progenitor cell.

43. The method of any one of claims 30-39, wherein the cell is an immune effector cell.

44. The method of any one of claim 30-39 or 43, wherein the cell is a lymphocyte.

45. The method of any one of claim 30-39, 43, or 44, wherein the cell is a T-lymphocyte.

46. A genetically engineered cell, wherein the cell is obtained or obtainable by the method of any of claims 30-45.

47. A cell population, comprising the genetically engineered cell of any one of claim 1-15 or 46.

48. A pharmaceutical composition comprising the genetically engineered hematopoietic cell, or descendant thereof, of any one of claims 1-15, the genetically engineered cell of claim 46, or the cell population of claim 47.

49. A genetically engineered hematopoietic stem cell, comprising a modification in a gene encoding CD34, wherein the genetically engineered hematopoietic stem cell does not express a naturally-occurring CD34 protein, and wherein the genetically engineered hematopoietic stem cell is functionally indistinguishable from a naturally occurring hematopoietic stem cell expressing CD34.

50. The genetically engineered hematopoietic stem cell of claim 49, wherein the genetically engineered hematopoietic stem cell expresses one or more hematopoietic stem cell markers.

51. The genetically engineered hematopoietic stem cell of any one of claim 49 or 50, wherein the genetically engineered hematopoietic stem cell expresses one or more of CD49c, CD71, CD90, CD117, CD135, CD201, CD228, CD243, CD292, CDw293, CD309, CD318, CD325, and CD349.

52. The genetically engineered hematopoietic stem cell of any one of claims 49-51, wherein the genetically engineered hematopoietic stem cell does not express CD34 on its cell surface.

53. The genetically engineered hematopoietic stem cell of any one of claims 49-52, wherein the genetically engineered hematopoietic stem cell does not express a CD34 epitope recognized by an anti-CD34 antibody on its cell surface.

54. The genetically engineered hematopoietic stem cell of any one of claims 49-52, wherein the genetically engineered hematopoietic stem cell does not express CD34 on its cell surface.

55. The genetically engineered hematopoietic stem cell of any one of claims 49-54, wherein the genetically engineered hematopoietic stem cell does not express CD34 on its cell surface and expresses one or more of CD49c, CD71, CD90, and CD201.

56. The genetically engineered hematopoietic stem cell of any one of claims 49-55, wherein the genetically engineered hematopoietic stem cell does not express CD34 on its cell surface and expresses one or more of CD49c, CD90, and CD201.

57. The genetically engineered hematopoietic stem cell of any one of claims 49-56, wherein the genetically engineered hematopoietic stem cell does not express a lineage-specific surface marker characteristic of differentiated hematopoietic cells (lin−).

58. The genetically engineered hematopoietic stem cell of any one of claims 49-57, wherein the genetically engineered hematopoietic stem cell does not express CD2, CD3, CD4, CD8, CD11b, CD14, CD15, CD16, CD19, CD56, CD123, or CD235a, or any combination thereof.

59. The genetically engineered hematopoietic stem cell of any one of claims 49-58, wherein the genetically engineered hematopoietic stem cell is capable of long-term engraftment into a human recipient.

60. The genetically engineered hematopoietic stem cell of any one of claims 49-58, wherein the genetically engineered hematopoietic stem cell is capable of reconstituting the hematopoietic system in a human recipient after engraftment.

61. The hematopoietic cell, or descendant thereof, of any one of claim 1-9 or 10-15, wherein the hematopoietic cell, or descendant thereof, lacks a CD34 epitope or has a modified CD34 epitope.
